(19) United States
(12) Patent Application Publication
(10) Pub. No.: US 2009/0118286 A1
(43) Pub. Date: May 7, 2009

(54) Heterobicyclic Acrylamides

(75) Inventors: Hans-Georg Schwarz, Langenfeld (DE); Jurgen Scherkenbeck, Wermelskirchen (DE); Graham Holmwood, Leverkusen (DE); Jurgen Wieschemeyer, Koln (DE); Peter Losel, Leverkusen (DE); Olga Malsam, Rosrath (DE); Mark Wilhelm Drewes, Langenfeld (DE); Karl-Heinz Kuck, Langenfeld (DE); Ulrike Wachendorf-Neumann, Neuwied (DE); Christian Arnold, Langenfeld (DE); Vincent Lee Salgado, Durham, NC (US); Thomas Bretschneider, Lohmar (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer Cropscience AG, Monheim (DE)

(21) Appl. No.: 11/817,860

(22) PCT Filed: Feb. 27, 2006
(86) PCT No.: PCT/EP2006/001761

§ 371 Date: Oct. 8, 2008

(30) Foreign Application Priority Data
Mar. 5, 2005 (DE) 10 2005 010 215.8

(51) Int. Cl. A01N 43/824; A01N 43/40; A01N 43/84; C07D 285/12; C07D 417/14; A01P 15/00

(52) U.S. Cl. 514/236.2; 514/363; 514/342; 548/136; 546/280.4; 544/134

(57) ABSTRACT

Use of heterobicyclic acrylamides of the formula (I)

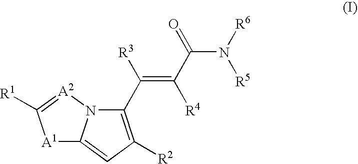

in which A¹, A², R¹, R², R³, R⁴, R⁵ and R⁶ are as defined in the description,
for controlling pests, novel heterobicyclic acrylamides and processes for preparing these compounds, and also compounds of the formula (IA)

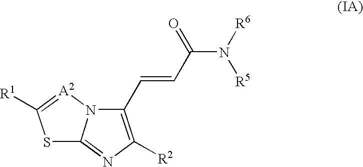

in which A², R¹, R², R⁵ and R⁶ are as defined in the description.

The present invention relates to the use of known and novel heterocyclic acrylamides and derivatives thereof as crop protection agents, in particular for controlling animals pests, to crop protection compositions based on known and novel heterobicyclic acrylamides and derivatives thereof, and to novel heterobicyclic acrylamides and derivatives thereof, and also to processes for their preparation.

Heterobicyclic acrylamides have already been described for use as medicaments (in particular as antihypertensive agents, diuretics and uricosurics) (cf. EP0189071 A2, EP0041215 A2, EP0088323 A2, EP0141072 A2; Sigrist et al., Developments in Nephrology 1987, 18 (diuretics), 547-549; Abdelal et al., Scientia Pharmaceutica, 1993, 61(1), 21-33; Akahane et al., Bioorganic & Med. Chem. Lett., 1996, 6(17), 2059-2062).

It has now been found that heterobicyclic acrylamides of the general formula (I) are highly effective against animal pests

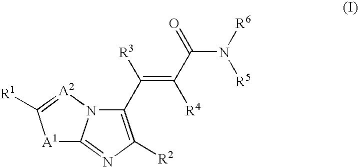

in which
A¹ represents sulphur, oxygen, NH or N-alkyl,
A² represents nitrogen or represents carbon which is substituted by hydrogen, halogen, hydroxyl, in each case straight-chain, branched or halogenated alkyl, by optionally halogenated alkoxy or by optionally halogenated cycloalkyl,
R¹ represents hydrogen, halogen, hydroxyl, cyano, nitro, represents optionally halogen-, hydroxyl-, cyano-, alkyl-, haloalkyl-, alkylamino-, dialkylamino- or alkoxy-substituted cycloalkyl, aryl, heteroaryl, represents optionally halogen-, hydroxyl-, cyano-, alkylamino-, dialkylamino- or alkoxy-substituted alkyl or represents optionally halogen-, cyano-, alkylamino- or dialkylamino-substituted alkoxy, alkylthio, alkylsulphinyl or alkylsulphonyl,
R² represents an aromatic or heteroaromatic radical which is mono- or polysubstituted by halogen, cyano, nitro, alkylamino, dialkylamino, alkyl, haloalkyl, alkoxy or phenoxy,
R³ represents hydrogen, alkyl or trifluoromethyl,
R⁴ represents hydrogen, cyano, trifluoromethyl, alkyl, halogen, alkoxy, nitro or SOₙ-alkyl, where n represents an integer from 0 to 2; represents straight-chain, branched or cyclic alkyl or represents the radical COR, where R represents hydrogen, hydroxyl, alkyl, alkoxy, aralkoxy, aryloxy or NR′R″ and R′ and R″ have the meanings given for R⁵ and R⁶,
R⁵ and R⁶ together or in each case on their own represent hydrogen, optionally substituted aryl, optionally substituted heteroaryl or represent a straight-chain, branched or cyclic, saturated or unsaturated aliphatic hydrocarbon radical which is optionally interrupted by O, S, N-alkyl, NH, N-aryl or N-aralkyl and which is in each case optionally substituted by hydroxyl, alkoxy, alkylmercapto, alkylsulphinyl, alkylsulphonyl, trialkylsilyl, alkyl, haloalkyl, halogen, optionally substituted phenyl, optionally substituted heteroaryl, alkoxycarbonyl, alkylamino or dialkylamino, where the last-mentioned dialkyl radicals together with the N-atom optionally form a 5- to 7-membered ring which is optionally interrupted by a heteroatom from the group consisting of O, S, NH and N-alkyl, and where these abovementioned alkylamino and dialkylamino radicals for their part are in each case optionally substituted by halogen, aryl, aralkyl, alkoxy, alkyl, haloalkyl, alkylmercapto or SO₂-alkyl,
   or
R⁵ and R⁶ together, bridged by the nitrogen atom, form a 3- to 7-membered saturated or unsaturated ring which optionally contains 1 or 2 further heteroatoms from the group consisting of oxygen, sulphur and nitrogen, where the nitrogen is optionally substituted by hydrogen, alkyl, aryl or aralkyl, where this 3- to 7-membered ring may be substituted by 1 to 4 identical or different substituents from the group consisting of alkyl, halogen, aryl, aralkyl, alkoxycarbonyl, hydroxyalkyl, alkoxyalkyl and trifluoromethyl, or where this 3- to 7-membered ring may be fused to an optionally substituted aromatic ring.

Preferred substituents or ranges of radicals listed in formula (I) are described below.
A¹ preferably represents sulphur, oxygen or NH,
A¹ particularly preferably represents sulphur or oxygen,
A¹ very particularly preferably represents sulphur.
A² preferably represents nitrogen or represents carbon which is substituted by hydrogen, halogen, hydroxyl, optionally mono- or polyhalogenated alkyl, optionally mono- or polyhalogenated alkoxy or optionally mono- or polyhalogenated cycloalkyl.
A² particularly preferably represents nitrogen or represents carbon which is substituted by hydrogen, halogen, hydroxyl, optionally mono- or polyhalogenated (C₁-C₆)-alkyl, optionally mono- or polyhalogenated (C₁-C₆)-alkoxy or optionally mono- or polyhalogenated (C₃-C₆)-cycloalkyl.
A² very particularly preferably represents nitrogen or represents carbon which is substituted by hydrogen, chlorine, fluorine, bromine, hydroxyl, methyl, ethyl, isopropyl, cyclopropyl, methoxy, ethoxy, trifluoromethoxy, trifluoromethyl or 1-chloro-1-cyclopropyl.
R¹ preferably represents hydrogen, halogen, hydroxyl, cyano, nitro or represents optionally halogen-, hydroxyl-, cyano-, alkyl-, haloalkyl-, alkylamino-, dialkylamino- or alkoxy-substituted cycloalkyl, represents optionally halogen-, hydroxyl-, cyano-, alkylamino-, dialkylamino- or alkoxy-substituted alkyl or represents optionally halogen-, cyano-, alkylamino- or dialkylamino-substituted alkoxy, alkylthio, alkylsulphinyl or alkylsulphonyl.
R¹ moreover preferably represents phenyl which is optionally mono- to pentasubstituted by halogen, hydroxyl, cyano, alkyl, alkoxy, haloalkyl, alkylamino or dialkylamino.
R¹ furthermore preferably represents heteroaryl which is optionally mono- to tetrasubstituted by halogen, hydroxyl, cyano, alkyl, alkoxy, haloalkyl, alkylamino or dialkylamino.
R¹ particularly preferably represents hydrogen, halogen, hydroxyl, cyano, nitro or represents in each case optionally halogen-, cyano-, (C₁-C₃)-alkylamino- and (C₁-C₃)-dialkylamino-substituted methoxy, ethoxy, isopropoxy, represents methylthio, methylsulphinyl, methylsulphonyl, ethylthio, ethylsulphinyl, ethylsulphonyl, represents methyl, ethyl, isopropyl, n-propyl, tert-butyl, methoxymethyl, methoxyethyl, cyclopropyl, cyclobutyl, cyclopentyl, cyclohexyl, fluoromethyl, chloromethyl, difluoromethyl, dichloromethyl, trifluoromethyl, difluorochloromethyl, trichloromethyl, fluorodichloromethyl, pentafluoroethyl, 2,2,2-trifluoroethyl, 1,1,1,3,3,3-hexafluoro-2-propyl, heptafluoroisopropyl or 1-chloro-1-cyclopropyl.
R¹ moreover particularly preferably represents phenyl which is optionally mono- to pentasubstituted by halogen, hydroxyl, cyano, alkyl, alkoxy, (C₁-C₃) haloalkyl, (C₁-C₃)-alkylamino or (C₁-C₃)-dialkylamino.
R¹ furthermore particularly preferably represents five- or six-membered heteroaryl which is optionally mono- to tetrasubstituted by halogen, hydroxyl, cyano, (C₁-C₃)-alkyl, (C₁-C₃) alkoxy, (C₁-C₃)-haloalkyl, (C₁-C₃)-alkylamino or (C₁-C₃)-dialkylamino.
R¹ very particularly preferably represents hydrogen, fluorine, chlorine, bromine, hydroxyl or represents methoxy, ethoxy, isopropoxy, methylthio, methylsulphinyl, methylsulphonyl, trifluoromethoxy, trichloromethoxy, difluoromethoxy, difluorochloromethoxy, fluorodichloromethoxy, methyl, ethyl, isopropyl, n-propyl, tert-butyl, methoxymethyl, methoxyethyl, cyclopropyl, cyclobutyl, cyclopentyl, cyclohexyl, fluoromethyl, chloromethyl, difluoromethyl, dichloromethyl, trifluoromethyl, difluorochloromethyl, trichloromethyl, fluorodichloromethyl, pentafluoroethyl, 2,2,2-trifluoroethyl, 1,1,1,3,3,3-hexafluoro-2-propyl, heptafluoroisopropyl or 1-chloro-1-cyclopropyl.
R¹ moreover very particularly preferably represents 4-chlorophenyl or 4-(trifluoromethyl)phenyl.
R² preferably represents phenyl which is optionally mono- to pentasubstituted by halogen, cyano, (C₁-C₃)-alkyl, (C₁-C₃)-alkoxy, phenoxy, (C₁-C₃)-haloalkyl, (C₁-C₃)-alkylamino or (C₁-C₃)-dialkylamino or represents up to six-membered heteroaryl which is mono- to tetrasubstituted by halogen, cyano, (C₁-C₃)-alkyl, (C₁-C₃)-alkoxy, phenoxy, (C₁-C₃)-haloalkyl, (C₁-C₃)-alkylamino or (C₁-C₃)-dialkylamino.
R² particularly preferably represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 3-methylphenyl, 4-methylphenyl, 2-chloro-4-methylphenyl, 2-methyl-4-chlorophenyl, 4-fluorophenyl, 2-methyl-4-fluorophenyl, 3,4-dichlorophenyl, 3,5-dichlorophenyl, 2,3-dichlorophenyl, 2,5-dichlorophenyl, 3,4-difluorophenyl, 3,5-difluorophenyl, 2,3-difluorophenyl, 2,5-difluorophenyl, 2,6-dichlorophenyl, 2,6-difluorophenyl, 2,3,4-trifluorophenyl, 3,4-bis(methoxy)phenyl, 4-methoxyphenyl, 3-methoxyphenyl, 2-methoxyphenyl, 2-chloro-4-methoxyphenyl, 2-fluoro-4-methoxyphenyl, 2-methyl-4-methoxyphenyl, 4-phenoxyphenyl, 2-chloro-4-phenoxyphenyl, 2-fluoro-4-phenoxyphenyl, 2-methyl-4-phenoxyphenyl, 4-(trifluoromethoxy)phenyl, 2-(trifluoromethyl)phenyl, 3-(trifluoromethyl)phenyl, 4-(trifluoromethyl)phenyl, 2-chloro-4-(trifluoromethyl)phenyl, 2-fluoro-4-(trifluoromethyl)phenyl, 4-(tert-butyl)phenyl, 4-cyanophenyl, thienyl, 3-chlorothien-2-yl, 4-chlorothien-2-yl, 5-chlorothien-2-yl, 2-chlorothien-4-yl, 3-chlorothien-4-yl or 2-chlorothien-3-yl, thiazol-2-yl, 5-chloro-4-thiazolyl, 2-chloro-3-pyridyl, 2-chloro-5-pyridyl, 3-bromo-5-pyridyl.
R² very particularly preferably represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 2-chloro-4-methylphenyl, 2-fluoro-4-methylphenyl, 2-methyl-4-chlorophenyl, 4-fluorophenyl, 2-methyl-4-fluorophenyl, 3,4-dichlorophenyl, 2,3-dichlorophenyl, 2,5-dichlorophenyl, 2,3-difluorophenyl, 2,5-difluorophenyl, 2,3,4-trifluorophenyl, 3,4-bis(methoxy)phenyl, 2-chloro-4-methoxyphenyl, 2-fluoro-4-methoxyphenyl, 2-methyl-4-methoxyphenyl, 3-(trifluoromethyl)phenyl, 2-chloro-4-(trifluoromethyl)phenyl, 2-fluoro-4-(trifluoromethyl)phenyl, 3-chlorothien-2-yl, 3-chlorothien-4-yl or 2-chlorothien-3-yl.
R³ preferably represents hydrogen, trifluoromethyl or (C₁-C₆)-alkyl.
R³ particularly preferably represents hydrogen.
R⁴ preferably represents hydrogen, cyano, trifluoromethyl, (C₁-C₆)-alkyl, nitro, CO—(C₁-C₆)-alkyl or SO₂—(C₁-C₆)-alkyl.
R⁴ particularly preferably represents hydrogen or cyano.
R⁴ very particularly preferably represents hydrogen.
R⁵ and R⁶ together or in each case on their own preferably represent hydrogen, alkyl, alkenyl, alkynyl, cycloalkyl, cycloalkylalkyl, alkoxyalkyl, di(alkoxy)-alkyl, alkylthioalkyl, alkylsulphinylalkyl, alkylsulphonylalkyl, alkylaminoalkyl, dialkylaminoalkyl, trialkylsilylalkyl, haloalkyl, haloalkenyl, haloalkynyl, halocycloalkyl, halocyloalkylalkyl, haloalkoxyalkyl, represent aryl, aralkyl, arylhaloalkyl, aryloxyalkyl, heteroaryl or heteroarylalkyl, each of which is mono- or polysubstituted by hydrogen, halogen, cyano, nitro, alkyl- or dialkylamino, haloalkyl, alkyl, haloalkoxy or alkoxy, or represent alkoxy, cycloalkoxy, cycloalkylalkoxy or arylalkoxy.
R⁵ and R⁶ together, bridged by the nitrogen atom, moreover preferably represent a 3- to 7-membered saturated or unsaturated ring which optionally contains 1 or 2 further heteroatoms from the group consisting of oxygen, sulphur and nitrogen, where the nitrogen is optionally substituted by hydrogen, (C₁-C₆)-alkyl, aryl or aralkyl and where this 3- to 7-membered ring may be substituted by 1 to 4 identical or different substituents from the group consisting of (C₁-C₆)-alkyl, (C₁-C₆)-haloalkyl, halogen, aryl, aralkyl, (C₁-C₆)-alkoxycarbonyl, (C₁-C₆)-alkoxyalkyl or where this 3- to 7-membered ring may be fused to an optionally substituted aromatic ring.
R⁵ and R⁶ together or in each case on their own particularly preferably represent hydrogen, (C₁-C₆)-alkyl, (C₂-C₆)-alkenyl, (C₂-C₆)-alkynyl, (C₃-C₆)-cycloalkyl, (C₃-C₆)-cycloalkyl-(C₁-C₄)-alkyl, (C₁-C₆)-alkoxy-(C₁-C₆)-alkyl, di-((C₁-C₆)-alkoxy)-(C₁-C₆)-alkyl, (C₁-C₆)-alkylthio-(C₁-C₆)-alkyl, (C₁-C₆)-alkylsulphinyl-(C₁-C₆)-alkyl, (C₁-C₆)-alkylsulphonyl-(C₁-C₆)-alkyl, (C₁-C₆)-alkylamino-(C₁-C₆)-alkyl, di-(C₁-C₆)-alkylamino-(C₁-C₆)-alkyl, (tri-(C₁-C₆)-alkyl)silyl-(C₁-C₆)-alkyl, represent (C₁-C₆)-alkyl, (C₂-C₆)-alkenyl, (C₂-C₆)-alkynyl, (C₃-C₆)-cycloalkyl, (C₃-C₆)-cycloalkyl-(C₁-C₄)-alkyl or (C₁-C₆)-alkoxy-(C₁-C₆)-alkyl, each of which is mono- or polysubstituted by fluorine, chlorine, bromine or iodine, represent aryl, aryl-(C₁-C₅)-alkyl, aryl-(C₁-C₅)-haloalkyl, aryloxy-(C₁-C₅)-alkyl, heteroaryl or heteroaryl-(C₁-C₅)-alkyl, each of which is mono- or polysubstituted by hydrogen, fluorine, chlorine, cyano, nitro, alkylamino, dialkylamino, haloalkyl, alkyl, haloalkoxy or alkoxy, or represent (C₁-C₆)-alkoxy, (C₃-C₆)-cycloalkoxy, (C₃-C₆)-cycloalkyl-(C₁-C₃)-alkoxy or aryl-(C₁-C₆)-alkoxy.
R⁵ and R⁶ together, bridged by the nitrogen atom, moreover particularly preferably represent in each case optionally fluorine-, chlorine-, bromine-, (C₁-C₆)-alkyl-, trifluoromethyl- or (C₁-C₆)-alkoxy-substituted piperidine, piperazine, morpholine or pyrrolidine.
R⁵ and R⁶ together or in each case on their own very particularly preferably represent hydrogen, methyl, ethyl, n-propyl, isopropyl, n-butyl, sec-butyl, isobutyl, tert-butyl, allyl, propargyl, cyclopropyl, cyclobutyl, cyclopentyl, cyclohexyl, cyclopropylmethyl, 1-pentyl, 2-pentyl, 3-pentyl, 2,2-dimethyl-1-propyl, 3-methyl-1-butyl, 3-dimethyl-1-butyl, 1,1-dimethyl-1-propyl, 1-methoxy-2-propyl, 2-methoxypropyl, 1-ethoxy-2-propyl, 2-ethoxypropyl, 2-methoxyethyl, 2-ethoxyethyl, 2-(methylthio)ethyl, 2-(ethylthio)ethyl, 2-(dimethylamino)ethyl, 2-(diethylamino)ethyl, 1-(dimethylamino)-2-propyl, 1-(diethylamino)-2-propyl, 1-(trimethylsilyl)ethyl, rac-1-methylthio-2-propyl, (S)-1-methylthio-2-propyl, (R)-1-methylthio-2-propyl, rac-1-methylsulphinyl-2-propyl, rac-2,2,2-trifluoromethyl-1-phenethyl, rac-2,2,2-trifluoromethyl-1-(4′-chloro)phenethyl, rac-2,2,2-trifluoromethyl-1-(4′-fluoro)phenethyl, 4,4,4-trifluoro-1-butyl, 1,1,1-trifluoro-2-methyl-2-propyl, (S)-1-methylsulphinyl-2-propyl, (R)-1-methylsulphinyl-2-propyl, rac-1-methylsulphonyl-2-propyl, (S)-1-methylsulphonyl-2-propyl, (R)-1-methylsulphonyl-2-propyl, 3-methyl-2-butyl, 2,2-bis(methoxy)ethyl, rac-1,1,1-trifluoro-2-propyl, (R)-1,1,1-trifluoro-2-propyl, 1,1,1-trifluoro-2-butyl, (S)-1,1,1-trifluoro-2-propyl, 1,1,1-trifluoro-3-methyl-2-butyl, 1,1,1-trifluoro-2-methyl-2-propyl, 1-(trifluoromethyl)-1-cyclopropyl, 2,2,2-trifluoroethyl, 3,3,3-trifluoro-1-propyl, 1,1,1,3,3,3-hexafluoro-2-propyl, heptafluoroisopropyl, 2,2,3,3,3-pentafluoro-1-propyl, 2,2,3,3,4,4,4-heptafluoro-1-butyl, 2-(trifluoromethyl)cyclohexyl, 3-(trifluoromethyl)cyclohexyl, 4-(trifluoromethyl)cyclohexyl, 1,1,1-trifluoro-3-phenyl-3-propyl, benzyl, 4-fluorobenzyl, 4-chlorobenzyl, 1-phenethyl, 1-(4-fluoro)phenethyl, 1-(4-chloro)phenethyl, 1-phenylpropyl, 1-(4-fluoro)phenylpropyl, 1-(4-chloro)phenylpropyl, 2-phenoxyethyl, 2-(4-fluoro)phenoxyethyl, 2-(4-chloro)phenoxyethyl, 1-phenoxy-2-propyl, 1-(4-fluoro)phenoxy-2-propyl, 1-(4-chloro)phenoxy-2-propyl, 1-cyclopropylethyl, 1-cyclobutylethyl, 1-cyclopentylethyl, 1-cyclohexylethyl, 1-cyclopropylpropyl, 1-cyclobutylpropyl, 1-cyclopentylpropyl, 1-cyclohexylpropyl, 2-phenethyl, 2-(4-fluoro)phenethyl, 2-(4-chloro)phenethyl, phenyl, 4-fluorophenyl, 4-chlorophenyl, 3-chlorophenyl, 2-chlorophenyl, 2,4-dichlorophenyl, 4-trifluoromethoxyphenyl, 4-trifluoromethylphenyl, (2-pyridyl)methyl, (2-pyridyl)-1-ethyl, (3-pyridyl)methyl, (3-pyridyl)-1-ethyl, (4-pyridyl)methyl, (4-pyridyl)-1-ethyl, (5-chloro-2-pyridyl)methyl, (5-chloro-2-pyridyl)-1-ethyl, [3-(trifluoromethyl)-5-chloro-2-pyridyl]-1-methyl, [3-(trifluoromethyl)-5-chloro-2-pyridyl]-1-ethyl, (2-chloro-5-pyridyl)methyl, (2-chloro-5-pyridyl)-1-ethyl, methoxy, ethoxy, isopropoxy, cyclopropylmethoxy, benzyloxy, 1-phenethoxy or 2-phenethoxy.
R⁵ and R⁶ together, bridged by the nitrogen atom, moreover very particularly preferably represent piperidine, piperazine, N-methylpiperazine, morpholine, 2,6-dimethylmorpholine, 2-(trifluoromethyl)piperidine, 3-(trifluoromethyl)piperidine, 4-(trifluoromethyl)piperidine or pyrrolidine.

Depending inter alia on the nature of the substituents, the compounds of the formula (I) may, if appropriate, be present as stereoisomers, i.e. as geometrical and/or optical isomers or isomer mixtures of varying compositions. The present invention provides both the pure stereoisomers and any mixtures of these isomers, even if, in general, only compounds of the formula (I) are referred to here.

The invention also provides the salt-like derivatives formed from compounds of the formula (I) by reaction with basic or acidic compounds.

The general or preferred radical definitions given above apply both to the end products of the formula (I) and, correspondingly, to the starting materials or intermediates required in each case for the preparation. These radical definitions can be combined with one another as desired, i.e. including combinations between the given preferred ranges.

Preference according to the invention is given to the compounds of the formula (I) which contain a combination of the meanings listed above as being preferred.

Particular preference according to the invention is given to the compounds of the formula (I) which contain a combination of the meanings given above as being particularly preferred.

Very particular preference according to the invention is given to the compounds of the formula (I) which contain a combination of the meanings given above as being very particularly preferred.

In the radical definitions given above and below, hydrocarbon radicals, such as alkyl, are in each case straight-chain or branched as far as this is possible—including in combination with heteroatoms, such as in alkoxy.

Some of the compounds of the general formula (I) to be used according to the invention are already known (cf. the literature cited above in the introduction).

Other compounds of the general formula (I) to be used according to the invention are novel.

Thus, the compounds of the general formula (IA)—as stated below—are claimed as novel compounds according to the invention.

A particular group of the compounds to be used according to the invention which, as novel compounds, are provided by this application, is thus represented by the formula (IA)

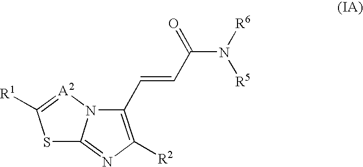

in which
A² represents nitrogen or represents carbon which is substituted by hydrogen, chlorine, fluorine, bromine, hydroxyl, methyl, ethyl, isopropyl, cyclopropyl, methoxy, ethoxy, trifluoromethoxy, trifluoromethyl or 1-chloro-1-cyclopropyl,
R¹ represents hydrogen, fluorine, chlorine, bromine, hydroxyl or represents methoxy, ethoxy, isopropoxy, methylthio, methylsulphinyl, methylsulphonyl, trifluoromethoxy, trichloromethoxy, difluoromethoxy, difluorochloromethoxy, fluorodichloromethoxy, methyl, ethyl, isopropyl, n-propyl, tert-butyl, methoxymethyl, methoxyethyl, cyclopropyl, cyclobutyl, cyclopentyl, cyclohexyl, fluoromethyl, chloromethyl, difluoromethyl, dichloromethyl, trifluoromethyl, difluorochloromethyl, trichloromethyl, fluorodichloromethyl, pentafluoroethyl, 2,2,2-trifluoroethyl, 1,1,1,3,3,3-hexafluoro-2-propyl, heptafluoroisopropyl, 1-chloro-1-cyclopropyl or 4-(trifluoromethyl)phenyl,
R² represents 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 3-methylphenyl, 4-methylphenyl, 2-chloro-4-methylphenyl, 2-fluoro-4-methylphenyl, 2-methyl-4-chlorophenyl, 4-fluorophenyl, 2-methyl-4-fluorophenyl, 3,4-dichlorophenyl, 3,5-dichlorophenyl, 2,3-dichlorophenyl, 2,5-dichlorophenyl, 3,4-difluorophenyl, 3,5-difluorophenyl, 2,3-difluorophenyl, 2,5-difluorophenyl, 2,6-dichlorophenyl, 2,6-difluorophenyl, 2,3,4-trifluorophenyl, 3,4-bis(methoxy)phenyl, 4-methoxyphenyl, 3-methoxyphenyl, 2-methoxyphenyl, 2-chloro-4-methoxyphenyl, 2-fluoro-4-methoxyphenyl, 2-methyl-4-methoxyphenyl, 4-phenoxyphenyl, 2-chloro-4-phenoxyphenyl, 2-fluoro-4-phenoxyphenyl, 2-methyl-4-phenoxyphenyl, 4-(trifluoromethoxy)phenyl, 2-(trifluoromethyl)phenyl, 3-(trifluoromethyl)phenyl, 4-(trifluoromethyl)phenyl, 2-chloro-4-(trifluoromethyl)phenyl, 2-fluoro-4-(trifluoromethyl)phenyl, 4-(tert-butyl)phenyl, 4-cyanophenyl, thienyl, 3-chlorothien-2-yl, 4-chlorothien-2-yl, 5-chlorothien-2-yl, 2-chlorothien-4-yl, 3-chlorothien-4-yl or 2-chlorothien-3-yl, thiazol-2-yl, 5-chloro-4-thiazolyl, 2-chloro-3-pyridyl, 2-chloro-5-pyridyl, 3-bromo-5-pyridyl,
R⁵ and R⁶ together or in each case on their own represent hydrogen, methyl, ethyl, n-propyl, isopropyl, n-butyl, sec-butyl, iso-butyl, tert-butyl, allyl, propargyl, cyclopropyl, cyclobutyl, cyclopentyl, cyclohexyl, cyclopropylmethyl, 1-pentyl, 2-pentyl, 3-pentyl, 2,2-dimethyl-1-propyl, 3-methyl-1-butyl, 3-dimethyl-1-butyl, 1,1-dimethyl-1-propyl, 1-methoxy-2-propyl, 2-methoxy-propyl, 1-ethoxy-2-propyl, 2-ethoxypropyl, 2-methoxyethyl, 2-ethoxyethyl, 2-(methylthio)ethyl, 2-(ethylthio)ethyl, 2-(dimethylamino)ethyl, 2-(diethylamino)ethyl, 1-(dimethylamino)-2-propyl, 1-(diethylamino)-2-propyl, 1-(trimethylsilyl)ethyl, rac-1-methylthio-2-propyl, (S)-1-methylthio-2-propyl, (R)-1-methylthio-2-propyl, rac-1-methylsulphinyl-2-propyl, (S)-1-methylsulphinyl-2-propyl, (R)-1-methylsulphinyl-2-propyl, rac-1-methylsulphonyl-2-propyl, rac-2,2,2-trifluoromethyl-1-phenethyl, rac-2,2,2-trifluoromethyl-1-(4′-chloro)phenethyl, rac-2,2,2-trifluoromethyl-1-(4′-fluoro)phenethyl, 4,4,4-trifluoro-1-butyl, 1,1,1-trifluoro-2-methyl-2-propyl, rac-2,2,2-trifluoromethyl-1-phenethyl, rac-2,2,2-trifluoromethyl-1-(4′-chloro)phenethyl, rac-2,2,2-trifluoromethyl-1-(4′-fluoro)phenethyl, 4,4,4-trifluoro-1-butyl, 1,1,1-trifluoro-2-methyl-2-propyl, (S)-1-methylsulphonyl-2-propyl, (R)-1-methylsulphonyl-2-propyl, 3-methyl-2-butyl, 2,2-bis(methoxy)ethyl, rac-1,1,1-trifluoro-2-propyl, (R)-1,1,1-trifluoro-2-propyl 1,1,1-trifluoro-2-butyl, (S)-1,1,1-trifluoro-2-propyl, 1,1,1-trifluoro-3-methyl-2-butyl, 1,1,1-trifluoro-2-methyl-2-propyl, 1-(trifluoromethyl)-1-cyclopropyl, 2,2,2-trifluoroethyl, 3,3,3-trifluoro-1-propyl, 1,1,1,3,3,3-hexafluoro-2-propyl, heptafluoroisopropyl, 2,2,3,3,3-pentafluoro-1-propyl, 2,2,3,3,4,4,4-heptafluoro-1-butyl, 2-(trifluoromethyl)cyclohexyl, 3-(trifluoromethyl)cyclohexyl, 4-(trifluoromethyl)cyclohexyl, 1,1,1-trifluoro-3-phenyl-3-propyl, benzyl, 4-fluorobenzyl, 4-chlorobenzyl, 1-phenethyl, 1-(4-fluoro)phenethyl, 1-(4-chloro)phenethyl, 1-phenylpropyl, 1-(4-fluoro)phenylpropyl, 1-(4-chloro)phenylpropyl, 2-phenoxyethyl, 2-(4-fluoro)phenoxyethyl, 2-(4-chloro)phenoxyethyl, 1-phenoxy-2-propyl, 1-(4-fluoro)phenoxy-2-propyl, 1-(4-chloro)phenoxy-2-propyl, 1-cyclopropylethyl, 1-cyclobutylethyl, 1-cyclopentylethyl, 1-cyclohexylethyl, 1-cyclopropylpropyl, 1-cyclobutylpropyl, 1-cyclopentylpropyl, 1-cyclohexylpropyl, 2-phenethyl, 2-(4-fluoro)phenethyl, 2-(4-chloro)phenethyl, phenyl, 4-fluorophenyl, 4-chlorophenyl, 3-chlorophenyl, 2-chlorophenyl, 2,4-dichlorophenyl, 4-trifluoromethoxyphenyl, 4-trifluoromethylphenyl, (2-pyridyl)methyl, (2-pyridyl)-1-ethyl, (3-pyridyl)methyl, (3-pyridyl)-1-ethyl, (4-pyridyl)methyl, (4-pyridyl)-1-ethyl, (5-chloro-2-pyridyl)methyl, (5-chloro-2-pyridyl)-1-ethyl, [3-(trifluoromethyl)-5-chloro-2-pyridyl]-1-methyl, [3-(trifluoromethyl)-5-chloro-2-pyridyl]-1-ethyl, (2-chloro-5-pyridyl)methyl, (2-chloro-5-pyridyl)-1-ethyl, methoxy, ethoxy, isopropoxy, cyclopropylmethoxy, benzyloxy, 1-phenethoxy or, 2-phenethoxy,
or
R⁵ and R⁶ together, bridged by the nitrogen atom, represent piperidine, piperazine, N-methylpiperazine, morpholine, 2,6-dimethylmorpholine, 2-(trifluoromethyl)piperidine, 3-(trifluoromethyl)piperidine, 4-(trifluoromethyl)piperidine or pyrrolidine.

Preferred substituents or ranges of the radicals given for formula (IA) are described below.
R² very particularly preferably represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 2-chloro-4-methylphenyl, 2-fluoro-4-methylphenyl, 2-methyl-4-chlorophenyl, 4-fluorophenyl, 2-methyl-4-fluorophenyl, 3,4-dichlorophenyl, 2,3-dichlorophenyl, 2,5-dichlorophenyl, 2,3-difluorophenyl, 2,5-difluorophenyl, 2,3,4-trifluorophenyl, 3,4-bis(methoxy)phenyl, 2-chloro-4-methoxyphenyl, 2-fluoro-4-methoxyphenyl, 2-methyl-4-methoxyphenyl, 3-(trifluoromethyl)phenyl, 2-chloro-4-(trifluoromethyl)phenyl, 2-fluoro-4-(trifluoromethyl)phenyl, 3-chlorothien-2-yl, 3-chlorothien-4-yl or 2-chlorothien-3-yl, thiazol-2-yl, 5-chloro-4-thiazolyl, 2-chloro-3-pyridyl, 2-chloro-5-pyridyl, 3-bromo-5-pyridyl.

The substituted heterobicyclic acrylamides of the general formula (I) have interesting biological properties. In particular, they have strong arthropodicidal (insecticidal and acaricidal) and nematicidal and fungicidal action and can be used in agriculture, in forests, in the protection of stored products and the protection of materials, and also in the hygiene sector.

Compounds of the general formula (I) can be synthesized as shown in the scheme below:

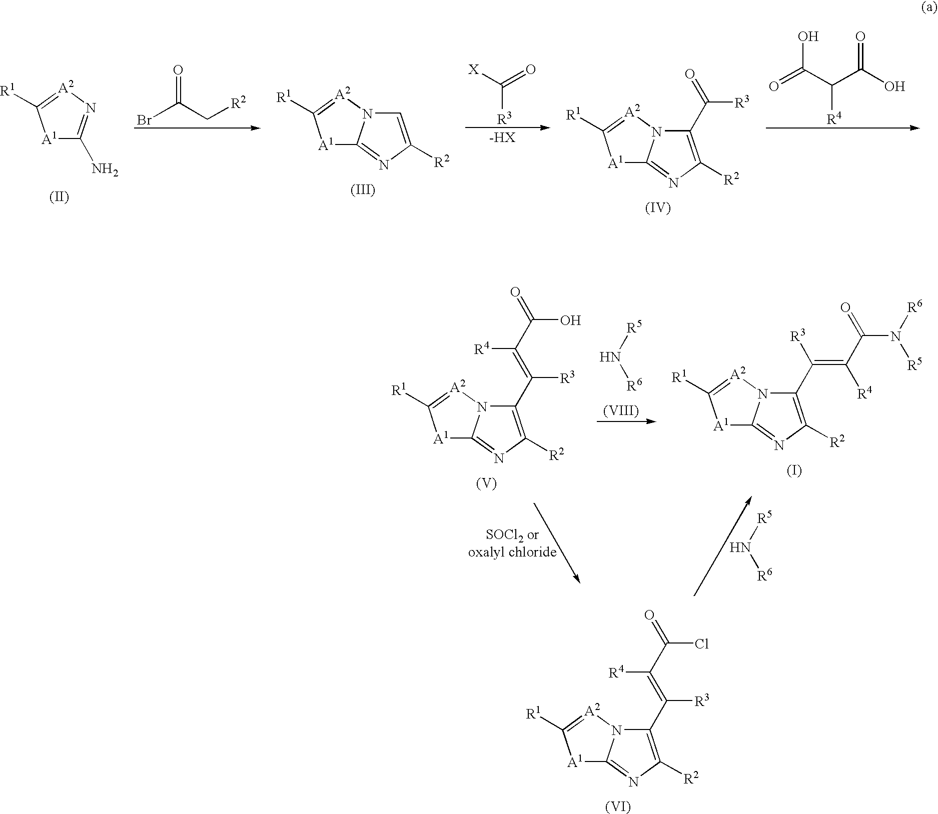

Alternatively, it is also possible to prepare the compounds of the formula (I) from compounds of the formula (VII):

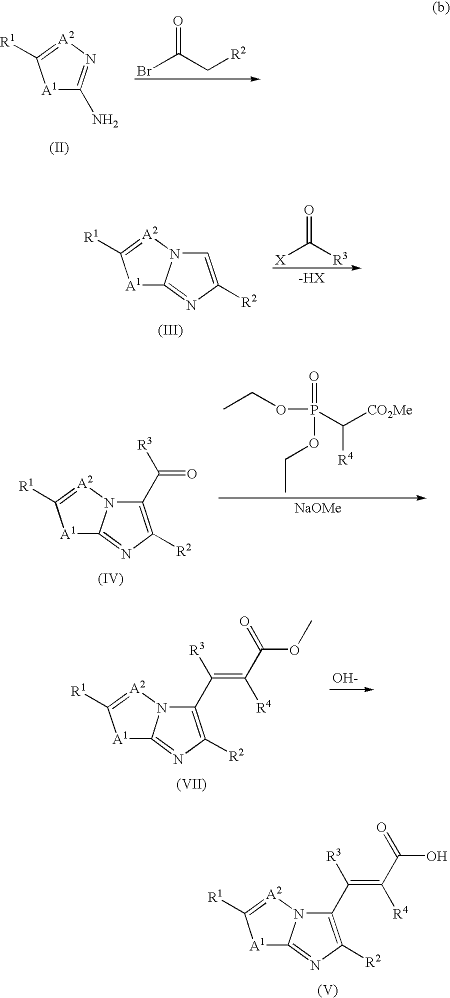

The formula (II) provides a general definition of the 1,3,4-thiadiazoles, 1,3,4-oxadiazoles, oxazoles, thiazoles and 1,3,4-triazoles further to be used as starting materials in the process according to the invention for preparing compounds of the general formula (I). In the general formulae (II), (III), (IV), (V), (VI) and (VII), the substituents A¹, A², R¹, R², R³, R⁴, R⁵ and R⁶ preferably have those meanings which have already been mentioned above, in connection with the description of the compounds of the general formula (I) according to the invention, as being preferred or as being particularly preferred for very particularly preferred for (1).

The starting materials or intermediates of the general formulae (II) to (VIII) are either known and/or can be prepared by processes known per set (cf. Preparation Examples). Starting materials of the general formula (II) are obtainable, for example, according to U.S. Pat. No. 4,264,616 A; Laduron et al., J. Fluorine Chem. 1995, 73(1), 83-86; Suyama et al., Heterocycles, 2003, 60(1), 121-129 or Abdel-Hafez et al., Arzneimittel-Forschung, 2002, 52(11), 833-839. The further synthesis steps of scheme (a) are described in principle in the literature references cited at the outset (EP0189071 A2, EP0041215 A2, EP0088323 A2, EP0141072 A2).

When carrying out the process according to the invention, the reaction temperatures can be varied within a relatively wide range. In general, the process is carried out at temperatures between 0° C. and 150° C., preferably between 10° C. and 120° C.

The process according to the invention is generally carried out under atmospheric pressure. However, it is also possible to carry out the process according to the invention under elevated or reduced pressure—in general between 0.1 bar and 10 bar.

For carrying out the process according to the invention, the starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours. Work-up is carried out by customary methods (cf. the Preparation Examples).

The compounds of the general formula (I) according to the invention can be converted into other compounds of the general formula (I) using methods known in principle. Some of these possible conversion reactions are illustrated in an exemplary manner below:

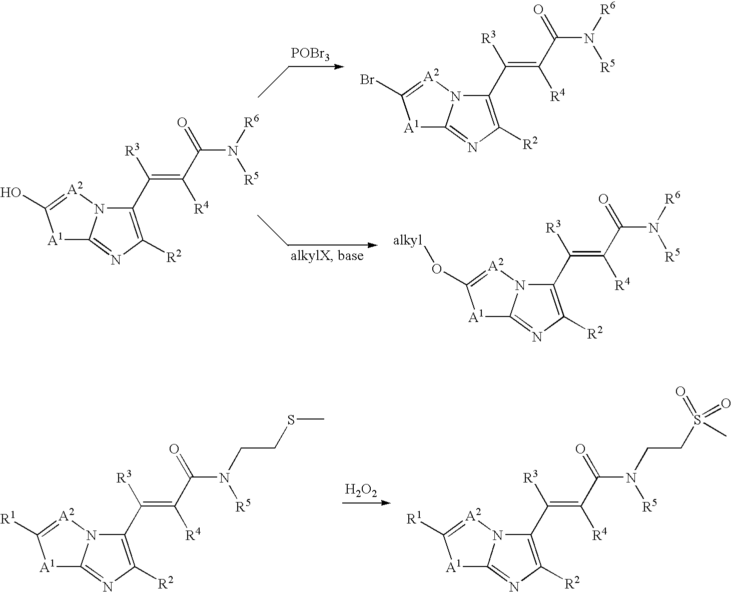

The compounds of the general formula (I) according to the invention are capable of forming salts. Suitable salts of the compounds of the general formula (I) which may be mentioned are customary nontoxic salts, i.e. salts with bases and salts (“adducts”) with acids. Preference is given to salts with inorganic bases, such as alkali metal salts, for example sodium, potassium or caesium salts, alkaline earth metal salts, for example calcium or magnesium salts, ammonium salts, salts with organic bases, in particular with organic amines, for example triethylammonium, dicyclohexylammonium, N,N′-dibenzylethylenediammonium, pyridinium, picolinium or ethanolammonium salts, salts with inorganic acids, for example hydrochlorides, hydrobromides, dihydrosulphates, trihydrosulphates, or phosphates, salts with organic carboxylic acids or organic sulphonic acids, for example formates, acetates, trifluoroacetates, maleates, tartrates, methanesulphonates, benzenesulphonates or paratoluenesulphonates.

Salts are formed according to the standard processes for preparing salts. The compounds according to the invention are, for example, reacted with appropriate acids to form acid addition salts. Representative acid addition salts are salts formed, for example, by reaction with inorganic acids, such as, for example, sulphuric acid, hydrochloric acid, hydrobromic acid, phosphoric acid, or organic carboxylic acids, such as acetic acid, trifluoroacetic acid, citric acid, succinic acid, lactic acid, formic acid, maleic acid, camphoric acid, phthalic acid, glycolic acid, glutaric acid, stearic acid, salicylic acid, sorbic acid, cinnamic acid, picric acid, benzoic acid, or organic sulphonic acids, such as methanesulphonic acid and para-toluenesulphonic acid.

The active compounds are suitable for protecting plants and plant organs, for increasing the harvest yields, for improving the quality of the harvested material and for controlling animal pests, in particular insects, arachnids and nematodes, which are encountered in agriculture, in forests, in gardens and leisure facilities, in the protection of stored products and of materials, and in the hygiene sector, and have good plant tolerance and favourable toxicity to warm-blooded animals and are tolerated well by the environment. They may be preferably employed as plant protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae, Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothripsfemoralis, Thrips tabaci, Thrips palmi and Frankliniella occidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Chematobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hyperapostica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

If appropriate, the compounds according to the invention can, at certain concentrations or application rates, also be used as herbicides or as safeners for herbicides, or microbicides, for example as fungicides, antimycotics and bactericides. If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, environment or storage space by the customary treatment methods, for example by immersion, spraying, evaporation, fogging, scattering, painting on, injection and, in the case of propagation material, in particular in the case of seeds, also by applying one or more coats.

The active compounds can be converted to the customary formulations, such as solutions, emulsions, wettable powders, suspensions, powders, dusts, pastes, soluble powders, granules, suspension-emulsion concentrates, natural and synthetic materials impregnated with active compound and microencapsulations in polymeric substances.

These formulations are produced in a known manner, for example by mixing the active compounds with extenders, that is liquid solvents, and/or solid carriers, optionally with the use of surfactants, that is emulsifiers and/or dispersants, and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

As solid carriers there are suitable:

for example ammonium salts and ground natural minerals such as kaolins, clays, talc, chalk quartz, attapulgite, montmorillonite or diatomaceous earth, and ground synthetic minerals, such as finely divided silica, alumina and silicates; as solid carriers for granules there are suitable: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; as emulsifiers and/or foam-formers there are suitable: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; as dispersants there are suitable: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The compounds according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:

Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
Pythium species, such as, for example, Pythium ultimum;
Phytophthora species, such as, for example, Phytophthora infestans;
Pseudoperonospora species, such as, for example, Pseudoperonospora humuli or Pseudoperonospora cubensis;
Plasmopara species, such as, for example, Plasmopara viticola;
Bremia species, such as, for example, Bremia lactucae;
Peronospora species, such as, for example, Peronospora pisi or P. brassicae;
Erysiphe species, such as, for example, Erysiphe graminis;
Sphaerotheca species, such as, for example, Sphaerotheca fuliginea;
Podosphaera species, such as, for example, Podosphaera leucotricha;
Venturia species, such as, for example, Venturia inaequalis;
Pyrenophora species, such as, for example, Pyrenophora teres or P. graminea (conidia form: Drechslera, syn: Helminthosporium);
Cochliobolus species, such as, for example, Cochliobolus sativus (conidia form: Drechslera, syn: Helminthosporium);
Uromyces species, such as, for example, Uromyces appendiculatus;
Puccinia species, such as, for example, Puccinia recondita;
Sclerotinia species, such as, for example, Sclerotinia sclerotiorum;
Tilletia species, such as, for example, Tilletia caries;
Ustilago species, such as, for example, Ustilago nuda or Ustilago avenae;
Pellicularia species, such as, for example, Pellicularia sasakii;
Pyricularia species, such as, for example, Pyricularia oryzae;
Fusarium species, such as, for example, Fusarium culmorum;
Botrytis species, such as, for example, Botrytis cinerea;
Septoria species, such as, for example, Septoria nodorum;
Leptosphaeria species, such as, for example, Leptosphaeria nodorum;
Cercospora species, such as, for example, Cercospora canescens;
Alternaria species, such as, for example, Alternaria brassicae; and
Pseudocercosporella species, such as, for example, Pseudocercosporella herpotrichoides.

The active compounds according to the invention also show a strong invigorating action in plants. Accordingly, they are suitable for mobilizing the internal defenses of the plant against attack by unwanted microorganisms.

In the present context, plant-invigorating (resistance-inducing) compounds are to be understood as meaning substances which are capable of stimulating the defense system of plants such that, when the treated plants are subsequently inoculated with unwanted microorganisms, they display substantial resistance to these microorganisms.

In the present case, unwanted microorganisms are to be understood as meaning phytopathogenic fungi, bacteria and viruses. The compounds according to the invention can thus be used to protect plants within a certain period of time after treatment against attack by the pathogens mentioned. The period of time for which this protection is achieved generally extends from 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention are also suitable for increasing the yield of crops. In addition, they show reduced toxicity and are well tolerated by plants.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be tackifiers, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular molds, wood-discoloring and wood-destroying fungi (Basidiomycetes) and against slime organisms and algae.

Microorganisms of the following genera may be mentioned as examples:

Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulfite waste liquors and methylcellulose.

Tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can be used in their commercially available formulations and in the use forms, prepared from these formulations, as a mixture with other active compounds, such as insecticides, attractants, sterilizing agents, bactericides, acaricides, nematicides, fungicides, growth-regulating substances or herbicides. The insecticides include, for example, phosphoric esters, carbamates, carboxylic esters, chlorinated hydrocarbons, phenylureas and substances produced by microorganisms, inter alia.

Particularly favourable mixing components are, for example, the following compounds:

### Fungicides:

2-phenylphenol; 8-hydroxyquinoline sulphate; acibenzolar-5-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulphide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipeonazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulphur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulphonyl)amino]-butanamide; 1-(1-naphthalenyl)-1H-pyrrole-2,5-dione; 2,3,5,6-tetrachloro-4-(methylsulphonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxaspiro-[4.5]decane-3-amine; sodium tetrathiocarbonate;
and copper salts and preparations, such as Bordeaux mixture; copper hydroxide; copper naphthenate; copper oxychloride; copper sulphate; cufraneb; cuprous oxide; mancopper; oxine-copper.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

### Insecticides/Acaricides/Nematicides:

### Acetylcholine Esterase (AChE) Inhibitors

1.1 Carbamates, for example
alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb

### 1.2 Triazamate

1.3 Organophosphates, for example
acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion

### Sodium Channel Modulators/Voltage-Gated Sodium Channel Blockers

2.1 Pyrethroids, for example
acrinathrin, allethrin (d-cis-trans, d-trans), beta-cyfluthrin, bifenthrin, bioallethrin, bioallethrin-S cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)

### 2.2 DDT

2.3 Oxadiazines, for example indoxacarb

### Acetylcholine Receptor Agonists/Antagonists

3.1 Chloronicotinyls, for example
acetamiprid, clothianidin, dinotefuran, imidacloprid, nitenpyram, nithiazine, thiacloprid, thiamethoxam
   3.2 Nicotine, bensultap, cartap

### Acetylcholine Receptor Modulators

4.1 Spinosyns, for example spinosad

### GABA-Gated Chloride Channel Antagonists

5.1 Cyclodiene organochlorines, for example
camphechlor, chlordane, endosulfan, gamma-HCH, HCH, heptachlor, lindane, methoxychlor
   5.2 Fiproles, for example
acetoprole, ethiprole, fipronil, vaniliprole

### Chloride Channel Activators

6.1 Mectins, for example
avermectin, emamectin, emamectin-benzoate, ivermectin, milbemycin

### Juvenile Hormone Mimetics

7.1 Juvenoids, for example
diofenolan, epofenonane, fenoxycarb, hydroprene, kinoprene, methoprene, pyriproxifen, triprene

### Ecdyson Agonists/Disruptors

8.1 Diacylhydrazines, for example
chromafenozide, halofenozide, methoxyfenozide, tebufenozide

### Chitin Biosynthesis Inhibitors

9.1 Benzoylureas, for example
bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron

### 9.2 Buprofezin

### 9.3 Cyromazine

### Oxidative Phosphorylation Inhibitors, ATP Disruptors

### 10.1 Diafenthiuron

10.2 Organotins, for example azocyclotin, cyhexatin, fenbutatin-oxide

### Oxidative Phosphorylation Decouplers Acting by Interrupting the H-Proton Gradient

11.1 Pyrroles, for example chlorfenapyr
11.2 Dinitrophenols, for example
binapacyrl, dinobuton, dinocap, DNOC

### Site-I Electron Transport Inhibitors

12.1 METIs, for example fenazaquin, fenpyroximate, pyrimidifen, pyridaben, tebufenpyrad, tolfenpyrad

### 12.2 Hydramethylnon

### 12.3 Dicofol

### Site-II Electron Transport Inhibitors

### 13.1 Rotenone

### Site-III Electron Transport Inhibitors

14.1 Acequinocyl, fluacrypyrim

### Microbial Disruptors of the Insect Gut Membrane

15.1 Bacillus thuringiensis strains

### Fat Synthesis Inhibitors

16.1 Tetronic acids, for example
spirodiclofen, spiromesifen
   16.2 Tetramic acids, for example
3-(2,5-dimethylphenyl)-8-methoxy-2-oxo-1-azaspiro[4.5]dec-3-en-4-yl ethyl carbonate (aka: carbonic acid, 3-(2,5-dimethylphenyl)-8-methoxy-2-oxo-1-azaspiro[4.5]dec-3-en-4-yl ethyl ester, CAS-Reg.-No.: 382608-10-8) and carbonic acid, cis-3-(2,5-dimethylphenyl)-8-methoxy-2-oxo-1-azaspiro[4.5]dec-3-en-4-yl ethyl ester (CAS-Reg.-No.: 203313-25-1)
   17.1 Carboxamides, for example flonicamid
   18.1 Octopaminergic agonists, for example amitraz
   19.1 Inhibitors of magnesium-stimulated ATPase, for example propargite
   20.1 BDCAs, for example N2-[1,1-dimethyl-2-(methylsulphonyl)ethyl]-3-iodo-N1-[2-methyl-4-[1,2,2,2-tetrafluoro-1-(trifluoromethyl)ethyl]phenyl]-1,2-benzenedicarboxamide (CAS-Reg.-No.: 272451-65-7)
   21.1 Nereistoxin analogues, for example thiocyclam hydrogen oxalate, thiosultap-sodium
   22.1 Biologicals, hormones or pheromones, for example
azadirachtin, Bacillus spec., Beauveria spec., codlemone, Metarrhizium spec., Paecilomyces spec., thuringiensin, Verticillium spec.
   Active Compounds with Unknown or Unspecific Mechanisms of Action
   23.1 Fumigants, for example
aluminium phosphide, methyl bromide, sulphuryl fluoride
   23.2 Selective antifeedants, for example
cryolite, flonicamid, pymetrozine
   23.3 Mite growth inhibitors, for example
clofentezine, etoxazole, hexythiazox
   23.4 amidoflumet, benclothiaz, benzoximate, bifenazate, bromopropylate, buprofezin, quinomethionate, chlordimeform, chlorobenzilate, chloropicrin, clothiazoben, cycloprene, cyflumetofen, dicyclanil, fenoxacrim, fentrifanil, flubendiamide, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin,
   and also products which comprise insecticidal plant extracts, nematodes, fungi or viruses.

A mixture with other known active compounds, such as herbicides, or with fertilizers and growth regulators, safeners and/or semiochemicals is also possible.

When used as insecticides in their commercially available formulations and in the use forms prepared with these formulations, the active compounds according to the invention can furthermore be present in the form of a mixture with synergists. Synergists are compounds by which the activity of the active compounds is increased without it being necessary for the synergist added to be active itself.

When used as insecticides in their commercially available formulations and in the use forms prepared with these formulations, the active compounds according to the invention can furthermore be present in the form of a mixture with inhibitors which reduce the degradation of the active compound after application in the habitat of the plant, on the surface of parts of plants or in plant tissues.

The active compound content of the use forms prepared from the commercially available formulations can vary within broad ranges. The active compound concentration of the use forms can be from 0.0000001 up to 95% by weight of active compound, preferably between 0.0001 and 1% by weight.

They are applied in a customary manner adapted to suit the use forms.

When used against hygiene pests and pests of stored products, the active compound is distinguished by excellent residual action on wood and clay as well as good stability to alkali on limed substrates.

As already mentioned above, it is possible to treat all plants and their parts in accordance with the invention. In a preferred embodiment, wild plant species or plant varieties and plant cultivars which have been obtained by traditional biological breeding methods, such as hybridization or protoplast fusion, and the parts of these varieties and cultivars are treated. In a further preferred embodiment, transgenic plants and plant cultivars which have been obtained by recombinant methods, if appropriate in combination with conventional methods (genetically modified organisms), and their parts are treated. The term “parts” or “parts of plants” or “plant parts” has been explained above.

Plants which are treated particularly preferably in accordance with the invention are those of the plant cultivars which are in each case commercially available or in use. Plant cultivars are understood as meaning plants with new traits which have been bred either by conventional breeding, by mutagenesis or by recombinant DNA techniques. They may take the form of cultivars, biotypes and genotypes.

Depending on the plant species or plant cultivars, their location and growth conditions (soils, climate, vegetation period, nutrition), the treatment according to the invention may also result in superadditive (“synergistic”) effects.

Thus, for example, reduced application rates and/or a widened activity spectrum and/or an increase in the activity of the substances and compositions which can be used in accordance with the invention, better plant growth, increased tolerance to high or low temperatures, increased tolerance to drought or to salinity in the water or soil, increased flowering performance, facilitated harvesting, accelerated maturation, higher yields, higher quality and/or better nutritional value of the harvested products, better storage characteristics and/or processability of the harvested products are possible which exceed the effects which were actually to be expected.

The preferred transgenic plants or plant cultivars (those obtained by recombinant methods) to be treated in accordance with the invention include all those plants which, owing to the process of recombinant modification, were given genetic material which confers particular, advantageous, valuable traits to these plants. Examples of such properties are better plant growth, increased tolerance to high or low temperatures, increased tolerance to drought or to salinity in the water or soil, increased flowering performance, facilitated harvesting, accelerated maturation, higher yields, higher quality and/or higher nutritional value of the harvested products, better storage characteristics and/or better processability of the harvested products. Further examples of such traits, examples which must be mentioned especially, are better defence of the plants against animal and microbial pests, such as against insects, mites, phytopathogenic fungi, bacteria and/or viruses and an increased tolerance of the plants to certain herbicidal active compounds. Examples of transgenic plants which may be mentioned are the important crop plants, such as cereals (wheat, rice), maize, soybeans, potato, cotton, tobacco, oilseed rape and fruit plants (with the fruits apples, pears, citrus fruits and grapes), with particular emphasis on maize, soybeans, potatoes, cotton, tobacco, and oilseed rape. Traits which are especially emphasized are the increased defence of the plants against insects, arachnids, nematodes and slugs and snails, owing to toxins being formed in the plants, in particular toxins which are generated in the plants by the genetic material of Bacillus thuringiensis (for example by the genes CryIA(a), CryIA(b), CryIA(c), CryIIA, CryIIIA, CryIIIB2, Cry9c Cry2Ab, Cry3Bb and CryIF and their combinations; hereinbelow “Bt plants”). Other traits which are particularly emphasized are the increased defence of plants against fungi, bacteria and viruses by the systemic acquired resistance (SAR), systemin, phytoalexins, elicitors and resistance genes and correspondingly expressed proteins and toxins. Other traits which are especially emphasized are the increased tolerance of the plants to certain herbicidal active compounds, for example imidazolinones, sulphonylureas, glyphosate or phosphinotricin (for example “PAT” gene). The genes which confer the desired traits in each case may also be present in the transgenic plants in combination with one another. Examples of “Bt plants” which may be mentioned are maize cultivars, cotton cultivars, soybean cultivars and potato cultivars which are commercially available under the trade names YIELD GARD® (for example maize, cotton, soybeans), KnockOut® (for example maize), StarLink® (for example maize), Bollgard® (cotton), Nucotn® (cotton) and NewLeaf® (potato). Examples of herbicide-tolerant plants which may be mentioned are maize cultivars, cotton cultivars and soybean cultivars which are commercially available under the trade names Roundup Ready® (tolerance to glyphosate, for example maize, cotton, soybean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties commercially available under the name Clearfield® (for example maize). Naturally, these statements also apply to plant cultivars having these genetic traits or genetic traits still to be developed, which plant cultivars will be developed and/or marketed in the future.

The plants listed can be treated particularly advantageously according to the invention with the compounds of the general formula I or the active compound mixtures according to the invention. The preferred ranges stated above for the active compounds and mixtures also apply to the treatment of these plants.

Particular emphasis may be given to the treatment of plants with the compounds or mixtures specifically mentioned in the present text.

The active compounds according to the invention are not only active against plant, hygiene and stored-product pests, but also, in the veterinary medicine sector, against animal parasites (ectoparasites), such as ixodid ticks, argasid ticks, scab mites, trombi-culid mites, flies (stinging and sucking), parasitic fly larvae, lice, hair lice, bird lice and fleas. These parasites include:

From the order of the Anoplurida, for example, Haematopinus spp., Linognathus spp., Pediculus spp., Phtirus spp., Solenopotes spp.

From the order of the Mallophagida and the sub-orders Amblycerina and Ischnocerina, for example, Trimenopon spp., Menopon spp., Trinoton spp., Bovicola spp., Werneckiella spp., Lepikentron spp., Damalina spp., Trichodectes spp and, Felicola spp.

From the order of the Diptera and the sub-orders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopyslla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattela germanica and Supella spp.

From the sub-class of the Acaria (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which attack agricultural livestock, such as, for example, cattle, sheep, goats, horses, pigs, donkeys, camels, buffaloes, rabbits, chickens, turkeys, ducks, geese, honeybees, other domestic animals, such as, for example, dogs, cats, cage birds, aquarium fish, and so-called experimental animals, such as, for example, hamsters, guinea-pigs, rats and mice. By combating these arthropods, it is intended to reduce deaths and decreased performances (in meat, milk, wool, hides, eggs, honey and the like), so that more economical and simpler animal keeping is made possible by using the active compounds according to the invention.

In the veterinary sector, the active compounds according to the invention are used in a known manner by enteral administration, for example in the form of tablets, capsules, drinks, drenches, granules, pastes, boli, the feed-through method, suppositories, by parenteral administration, such as, for example, by means of injections (intramuscular, subcutaneous, intravenous, intraperitoneal and the like), implants, by nasal application, by dermal administration, for example in the form of dipping or bathing, spraying, pouring-on and spotting-on, washing, dusting, and with the aid of shaped articles which comprise active compound, such as collars, ear tags, tail marks, limb bands, halters, marking devices and the like.

When administered to livestock, poultry, domestic animals and the like, the active compounds of the formula (I) can be used as formulations (for example powders, emulsions, flowables) which comprise the active compounds in an amount of 1 to 80% by weight, either directly or after dilution by a factor of 100 to 10 000, or they may be used in the form of a chemical bath.

Furthermore, it has been found that the compounds according to the invention have a potent insecticidal action against insects which destroy industrial materials.

The following insects may be mentioned by way of example and as being preferred, but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus;
Dermapterans, such as Sirex juvencus, Urocerus gigas, Urocerus gigas taignus, Urocerus augur;
Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;
Bristletails, such as Lepisma saccharina.

Industrial materials are to be understood as meaning, in the present context, non-live materials, such as, preferably, synthetic materials, glues, sizes, paper and board, leather, wood and timber products, and paint.

The materials to be very particularly preferably protected against attack by insects are wood and timber products.

Wood and timber products which can be protected by the composition according to the invention or mixtures comprising such a composition are to be understood as meaning, for example:

construction timber, wooden beams, railway sleepers, bridge components, jetties, wooden vehicles, boxes, pallets, containers, telephone poles, wood cladding, windows and doors made of wood, plywood, particle board, joiner's articles, or wood products which, quite generally, are used in the construction of houses or in joinery.

The active compounds can be used as such, in the form of concentrates or generally customary formulations, such as powders, granules, solutions, suspensions, emulsions or pastes.

The formulations mentioned can be prepared in a manner known per se, for example by mixing the active compounds with at least one solvent or diluent, emulsifier, dispersant and/or binder or fixative, water repellent, if appropriate desiccants and UV stabilizers and, if appropriate, colorants and pigments and other processing auxiliaries.

The insecticidal compositions or concentrates used for the protection of wood and wooden materials comprise the active compound according to the invention in a concentration of 0.0001 to 95% by weight, in particular 0.001 to 60% by weight.

The amount of the compositions or concentrates employed depends on the species and the occurrence of the insects and on the medium. The optimum rate of application can be determined upon use in each case by a test series. However, in general, it suffices to employ 0.0001 to 20% by weight, preferably 0.001 to 10% by weight, of the active compound, based on the material to be protected.

The solvent and/or diluent used is an organochemical solvent or solvent mixture and/or an oily or oil-type organochemical solvent or solvent mixture of low volatility and/or a polar organochemical solvent or solvent mixture and/or water and, if appropriate, an emulsifier and/or wetting agent.

Organochemical solvents which are preferably employed are oily or oil-type solvents having an evaporation number of above 35 and a flashpoint of above 30° C., preferably above 45° C. Substances which are used as such oily and oil-type solvents which have low volatility and are insoluble in water are suitable mineral oils or their aromatic fractions, or mineral-oil-containing solvent mixtures, preferably white spirit, petroleum and/or alkylbenzene.

Substances which are advantageously used are mineral oils with a boiling range of 170 to 220° C., white spirit with a boiling range of 170 to 220° C., spindle oil with a boiling range of 250 to 350° C., petroleum or aromatics of boiling range 160 to 280° C., essence of terpentine and the like.

In a preferred embodiment, liquid aliphatic hydrocarbons with a boiling range of 180 to 210° C. or high-boiling mixtures of aromatic and aliphatic hydrocarbons with a boiling range of 180 to 220° C. and/or spindle oil and/or monochloronaphthalene, preferably α-monochloronaphthalene, are used.

The organic oily or oil-type solvents of low volatility having an evaporation number of above 35 and a flashpoint of above 30° C., preferably above 45° C., can be partially replaced by organochemical solvents of high or medium volatility, with the proviso that the solvent mixture also has an evaporation number of above 35 and a flashpoint of above 30° C., preferably above 45° C., and that the insecticide/fungicide mixture is soluble or emulsifiable in this solvent mixture.

In a preferred embodiment, part of the organochemical solvent or solvent mixture or an aliphatic polar organochemical solvent or solvent mixture is replaced. Substances which are preferably used are aliphatic organochemical solvents having hydroxyl and/or ester and/or ether groups, such as, for example, glycol ethers, esters and the like.

The organochemical binders used within the scope of the present invention are the synthetic resins and/or binding drying oils which are known per se and can be diluted with water and/or are soluble or dispersible or emulsifiable in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin, such as indene/cumarone resin, silicone resin, drying vegetable and/or drying oils and/or physically drying binders based on a natural and/or synthetic resin.

The synthetic resin used as the binder can be employed in the form of an emulsion, dispersion or solution. Up to 10% by weight of bitumen or bituminous substances can also be used as binders. In addition, colorants, pigments, water repellents, odour-masking substances and inhibitors or anticorrosives known per se and the like addition be employed.

The composition or the concentrate preferably comprises, in accordance with the invention, at least one alkyd resin or modified alkyd resin and/or a drying vegetable oil as the organochemical binder. Preferably used according to the invention are alkyd resins with an oil content of over 45% by weight, preferably 50 to 68% by weight.

All or some of the abovementioned binder can be replaced by a fixative (mixture) or a plasticizer (mixture). These additives are intended to prevent volatilization of the active compounds and crystallization or precipitation. They preferably replace 0.01 to 30% of the binder (based on 100% of binder employed).

The plasticizers are from the chemical classes of the phthalic esters, such as dibutyl phthalate, dioctyl phthalate or benzyl butyl phthalate, the phosphoric esters, such as tributyl phosphate, the adipic esters, such as di-(2-ethylhexyl) adipate, the stearates, such as butyl stearate or amyl stearate, the oleates, such as butyl oleate, the glycerol ethers or relatively high-molecular-weight glycol ethers, glycerol esters and p-toluenesulphonic esters.

Fixatives are chemically based on polyvinyl alkyl ethers, such as, for example, polyvinyl methyl ether, or ketones, such as benzophenone or ethylenebenzophenone.

Particularly suitable as a solvent or diluent is also water, if appropriate as a mixture with one or more of the abovementioned organochemical solvents or diluents, emulsifiers and dispersants.

Particularly effective protection of wood is achieved by large-scale industrial impregnation processes, for example vacuum, double-vacuum or pressure processes.

If appropriate, the ready-to-use compositions can additionally comprise other insecticides and, if appropriate, additionally one or more fungicides.

Suitable additional components which may be admixed are, preferably, the insecticides and fungicides mentioned in WO 94/29 268. The compounds mentioned in that document are expressly part of the present application.

Very particularly preferred components which may be admixed are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxid, triflumuron, chlothianidin, spinosad, tefluthrin,

and fungicides, such as epoxyconazole, hexaconazole, azaconazole, propiconazole, tebuconazole, cyproconazole, metconazole, imazalil, dichlorfluanid, tolylfluanid, 3-iodo-2-propynylbutyl carbamate, N-octyl-isothiazolin-3-one and 4,5-dichloro-N-octylisothiazolin-3-one.

The compounds according to the invention can at the same time be employed for protecting objects which come into contact with salt water or brackish water, in particular hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl(2-phenyl-4-chlorophenoxy)tin, tributyltin oxide, molybdenum disulphide, antimony oxide, polymeric butyl titanate, phenyl(bispyridine)bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;
fungicides such as benzo[b]thiophenecarboxylic acid cyclohexylamide S,S-dioxide, dichlofluanid, fluorfolpet, 3-iodo-2-propinyl butylcarbamate, tolylfluanid and azoles such as azaconazole, cyproconazole, epoxyconazole, hexaconazole, metconazole, propiconazole and tebuconazole;
molluscicides such as fentin acetate, metaldehyde, methiocarb, niclosamid, thiodicarb and trimethacarb, Fe chelates;
or conventional antifouling active compounds such as 4,5-dichloro-2-octyl-4-isothiazolin-3-one, diiodomethylparatryl sulphone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)pyridine, 2,4,5,6-tetrachloroisophthalonitrile, tetramethylthiuram disulphide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as rosin to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

A mixture with other known active compounds such as herbicides, or with fertilizers and growth regulators, safeners or semiochemicals is also possible.

In addition, the compounds of the formula (I) according to the invention also have very good antimycotic activity. They have a very broad antimycotic spectrum of action, in particular against dermatophytes and budding fungi, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The enumeration of these fungi is no restriction whatsoever of the mycotic spectrum which can be controlled and is provided by illustration only.

The active compounds can be employed as such, in the form of their formulations or the use forms prepared therefrom, such as ready-to-use solutions, suspensions, wettable powders, pastes, soluble powders, dusts and granules. They are applied in the customary manner, for example by pouring, spraying, atomizing, broadcasting, dusting, foaming, painting on and the like. It is furthermore possible to apply the active compounds by the ultra-low volume method, or to inject the active compound preparation or the active compound itself into the soil. The seeds of the plants can also be treated.

When employing the active compounds according to the invention as fungicides, the application rates can be varied within a substantial range, depending on the type of application. In the treatment of plant parts, the application rates of active compound are generally between 0.1 and 10 000 g/ha, preferably between 10 and 1000 g/ha. For the treatment of seed, the application rates of active compound are generally between 0.001 and 50 g per kilogram of seed, preferably between 0.01 and 10 g per kilogram of seed. For treating the soil, the application rates of active compound are generally between 0.1 and 10 000 g/ha, preferably between 1 and 5000 g/ha.

### PREPARATION EXAMPLES

### Example No. A

### Step 1 [Intermediate According to Formula (III), Example (III-1)]

### 6-(2,4-Dichlorophenyl)-2-(trifluoromethyl)imidazo[2,1-b][1,3,4]thiadiazole

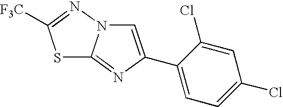

15.8 g (93.4 mmol) of 5-(trifluoromethyl)-1,3,4-thiadiazole-2-amine are initially charged in 100 ml of p.A.-grade ethanol. At room temperature (about 20° C.), a solution of 25.0 g (93.4 mmol) of 2-bromo-1-(2,4-dichlorophenyl)ethanone in 50 ml of p.A.-grade ethanol is added dropwise. The reaction mixture is heated at reflux for 8 h. The mixture is then cooled to −5° C. The precipitated solid is filtered off with suction and dried.

This gives 26.7 g (84.5% of theory) of 6-(2,4-dichlorophenyl)-2-(trifluoromethyl)imidazo[2,1-b][1,3,4]thiadiazole, ¹H-NMR (400 MHz, d₆-DMSO), δ 8.97 (s, 1H), 8.14-8.11 (d, 1H), 7.73 (d, 1H), 7.57-7.55 (dd, 1H).

### Step 2 [Intermediate According to Formula (IV), Example (IVA-1)]

### 6-(2,4-Dichlorophenyl)-2-(trifluoromethyl)imidazo[2,1-b][1,3,4]thiadiazole-5-carbaldehyde

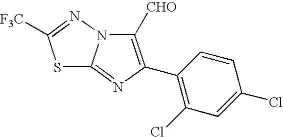

At 5° C., 68.6 g (0.447 mol) of phosphoryl chloride are added dropwise to a solution of 37.8 g (0.118 mol) of 6-(2,4-dichlorophenyl)-2-(trifluoromethyl)imidazo[2,1-b][1,3,4]thiadiazole in 150 ml of DMF. The mixture is stirred at 5° C. for 15 min and then heated at 100° C. for 2 h. The reaction mixture is introduced into 500 ml of ice-water and then neutralized using 5 N NaOH and extracted repeatedly with dichloromethane. The combined organic phases are dried over sodium sulphate, filtered and concentrated on a rotary evaporator. The crude product is chromatographed on silica gel (dichloromethane:cyclohexane=90:10% by volume), and the corresponding eluate is concentrated on a rotary evaporator and digested with methyl tert-butyl ether. The resulting crystalline solid is filtered off with suction and dried.

This gives 13.5 g (33% of theory) of 6-(2,4-dichlorophenyl)-2-(trifluoromethyl)imidazo[2,1-b][1,3,4]thiadiazole-5-carbaldehyde, ¹H-NMR (400 MHz, d₆-DMSO), δ 9.79 (s, 1H), 7.85 (s, 1H), 7.70 (d, 1H), 7.62 (d, 1H).

### Step 3 [Intermediate According to Formula (V), Example (VA-1)]

### (2E)-3-[6-(2,4-Dichlorophenyl)-2-(trifluoromethyl)imidazo[2,1-b][1,3,4]thiadiazol-5-yl]acrylic Acid

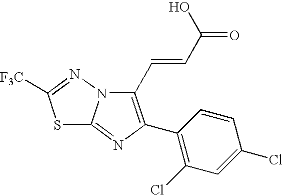

13.5 g (36.9 mmol) of 6-(2,4-dichlorophenyl)-2-(trifluoromethyl)imidazo[2,1-b][1,3,4]thiadiazole-5-carbaldehyde are suspended in 100 ml of pyridine, 2.84 ml of piperidine are added at room temperature (about 20° C.) and the mixture is then stirred at room temperature for 10 minutes. After addition of 4.6 g (44.2 mmol) of malonic acid, the mixture is heated at 100° C. for 2 h. The reaction mixture is poured into 300 ml of water and the precipitated solid is filtered off with suction; the filtercake is washed with petroleum ether:methyl tert-butyl ether=50:50% by volume and dried.

This gives 12.7 g (79.4% of theory) of (2E)-3-[6-(2,4-dichlorophenyl)-2-(trifluoromethyl)imidazo[2,1-b][1,3,4]thiadiazol-5-yl]acrylic acid, log P=3.15^{a)}, ¹H-NMR (400 MHz, d₆-DMSO), δ 12.61-12.49 (broad s, 1H), 7.85 (s 1H), 7.64-7.57 (2d, 2H), 7.33-7.30 (d, 1H, ³J=15.9), 6.71-6.67 (d, 1H, ³J=15.9).

### Step 4 [Active Compound According to Formula (IA), Example 0001]

### (2E)-3-[6-(2,4-Dichlorophenyl)-2-(trifluoromethyl)imidazo[2,1-b][1,3,4]thiadiazol-5-yl]-N-[(1S)-1-methyl-2-(methylthio)ethyl]acrylamide

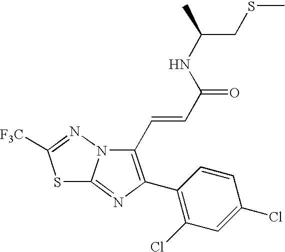

3.0 g (7.35 mmol) of (2E)-3-[6-(2,4-dichlorophenyl)-2-(trifluoromethyl)imidazo[2,1-b][1,3,4]thiadiazol-5-yl]acrylic acid are suspended in 50 ml dichloromethane, and two drops of DMF are added. 0.71 ml (8.1 mmol) of oxalyln chloride is added dropwise, and a strong evolution of gas is observed. The reaction mixture is stirred at 35° C. for 90 min and then directly concentrated on a rotary evaporator and taken up in 50 ml of tetrahydrofuran. The THF solution of the acid chloride generated in this manner is, at room temperature (about 20° C.), added dropwise to a solution of 1.1 g (8.1 mmol) of (2S)-1-(methylthio)propane-2-amine and 1.13 ml (8.1 mmol) of triethylamine in tetrahydrofuran. The mixture is stirred at 60° C. for 2 h and then poured into water and extracted repeatedly with dichloromethane. The combined organic phases are dried over sodium sulphate, filtered and concentrated on a rotary evaporator. The residue that remains is stirred with petroleum ether, filtered off with suction and dried.

This gives 2.8 g (73.8% of theory) of (2E)-3-[6-(2,4-dichlorophenyl)-2-(trifluoromethyl)imidazo[2,1-b][1,3,4]thiadiazol-5-yl]-N-[(1S)-1-methyl-2-(methylthio)ethyl]acrylamide, log P=3.91^{a)}.

### Step 5.1 [Active Compound According to Formula (IA, Example 0002]

### (2E)-3-[6-(2,4-Dichlorophenyl)-2-(trifluoromethyl)imidazo[2,1-b][1,3,4]thiadiazol-5-yl]-N-[(1S)-1-methyl-2-(methylsulphinyl)ethyl]acrylamide

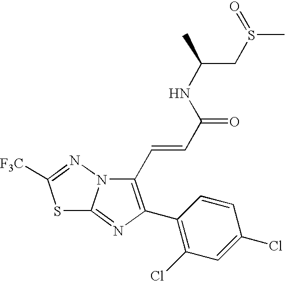

1.0 g (2 mmol) of (2E)-3-[6-(2,4-dichlorophenyl)-2-(trifluoromethyl)imidazo[2,1-b][1,3,4]-thiadiazol-5-yl]-N-[(1S)-1-methyl-2-(methylthio)ethyl]acrylamide is dissolved in 30 ml of dichloromethane, and 0.45 g (2 mmol) of 3-chloroperoxybenzoic acid is added. The solution is stirred at room temperature (about 20° C.) for 2 h and then washed with saturated aqueous NaHCO₃ solution and water, dried over sodium sulphate and concentrated on a rotary evaporator. The product is separated chromatographically from the sulphone by-product (dichloromethane:ethyl acetate=95:5% by volume).

This gives 0.7 g (63.5% of theory) of (2E)-3-[6-(2,4-dichlorophenyl)-2-(trifluoromethyl)imidazo[2,1-b][1,3,4]thiadiazol-5-yl]-N-[(1S)-1-methyl-2-(methylsulphinyl)ethyl]acrylamide, log P=2.48^{a)}.

### Step 5.2 [Active Compound According to Formula (IA), Example 0003]

### (2E)-3-[6-(2,4-Dichlorophenyl)-2-(trifluoromethyl)imidazo[2,1-b][1,3,4]thiadiazol-5-yl]-N-[(1S)-1-methyl-2-(methylsulphonyl)ethyl]acrylamide

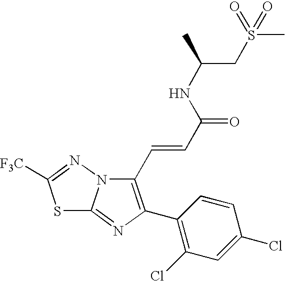

0.7 g (1.4 mmol) of (2E)-3-[6-(2,4-dichlorophenyl)-2-(trifluoromethyl)imidazo[2,1-b][1,3,4]-thiadiazol-5-yl]-N-[(1S)-1-methyl-2-(methylthio)ethyl]acrylamide is dissolved in 10 ml of glacial acetic acid, and 0.16 g (1.4 mmol) of 30% strength aqueous hydrogen peroxide solution is added. The reaction mixture is then heated at 80° C. for 2 h and subsequently poured into 100 ml of water and extracted repeatedly with dichloromethane. The combined organic phases are dried over sodium sulphate, filtered and concentrated on a rotary evaporator. The product is purified by column chromatography (dichloromethane:acetonitrile=90:10% by volume).

This gives 0.1 g (13.4% of theory) of (2E)-3-[6-(2,4-dichlorophenyl)-2-(trifluoromethyl)imidazo[2,1-b][1,3,4]thiadiazol-5-yl]-N-[(1S)-1-methyl-2-(methylsulphonyl)ethyl]acrylamide, log P=3.03^{a)}. ^{a)} The determination of the log P values was carried out in accordance with EEC Directive 79/831 Annex V.A8 by HPLC (gradient method, acetonitrile/0.1% strength aqueous phosphoric acid or formic acid).

### Further Starting Materials:

The starting materials of the formulae (III)-(V) can be prepared as above and as in the literature described therein. Examples of the formulae (IVA)-(VA) are listed in Tables 2 and 1.

TABLE 1

| | |
|---|---|
| | (VA) |
| 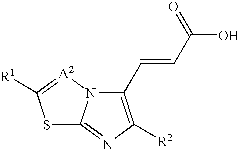 | |

| | | | | Physical |
|---|---|---|---|---|
| Ex. No. | A² | R¹ | R² | Data (logP)* |
| VA-1 | N | CF₃ | 2,4-C1₂-phenyl | 3.15 |
| VA-2 | N | CF₃ | 4-Cl-phenyl | |
| VA-3 | N | CF₃ | 3-Cl-phenyl | |
| VA-4 | N | CF₃ | 2-Cl-phenyl | |
| VA-5 | N | CF₃ | 2-CH₃-phenyl | 2.77 |
| VA-6 | N | CF₃ | 4-F-phenyl | 2.75 |
| VA-7 | N | CF₃ | 3-CF₃-phenyl | 3.33 |
| VA-8 | N | CF₃ | 2-F-phenyl | 2.55 |
| VA-9 | N | CF₃ | 2-Cl-4-F-phenyl | 2.84 |
| VA-10 | N | CF₃ | 3,4-(OCH₃)₂-phenyl | 2.43 |
| VA-11 | N | CHF₂ | 2,4-Cl₂-phenyl | |
| VA-12 | N | CHF₂ | 4-Cl-phenyl | |
| VA-13 | N | CHF₂ | 3-Cl-phenyl | |
| VA-14 | N | CHF₂ | 2-Cl-phenyl | 2.41 |
| VA-15 | N | CHF₂ | 2-CH₃-phenyl | |
| VA-16 | N | CHF₂ | 4-F-phenyl | |
| VA-17 | N | CHF₂ | 3-CF₃-phenyl | |
| VA-18 | N | CHF₂ | 2-F-phenyl | |
| VA-19 | N | CHF₂ | 2-Cl-4-F-phenyl | |
| VA-20 | N | CHF₂ | 3,4-(OCH₃)₂-phenyl | |
| VA-21 | N | CClF₂ | 2,4-Cl₂-phenyl | |
| VA-22 | N | CClF₂ | 4-Cl-phenyl | |
| VA-23 | N | CClF₂ | 3-Cl-phenyl | |
| VA-24 | N | CClF₂ | 2-Cl-phenyl | 2.93 |
| VA-25 | N | CClF₂ | 2-CH₃-phenyl | |
| VA-26 | N | CClF₂ | 4-F-phenyl | |
| VA-27 | N | CClF₂ | 3-CF₃-phenyl | |
| VA-28 | N | CClF₂ | 2-F-phenyl | |
| VA-29 | N | CClF₂ | 2-Cl-4-F-phenyl | |
| VA-30 | N | CClF₂ | 3,4-(OCH₃)₂-phenyl | |
| VA-31 | N | CCl₂F | 2,4-Cl₂-phenyl | |
| VA-32 | N | CCl₂F | 4-Cl-phenyl | |
| VA-33 | N | CCl₂F | 3-Cl-phenyl | |
| VA-34 | N | CCl₂F | 2-Cl-phenyl | |
| VA-35 | N | CCl₂F | 2-CH₃-phenyl | |
| VA-36 | N | CCl₂F | 4-F-phenyl | |
| VA-37 | N | CCl₂F | 3-CF₃-phenyl | |
| VA-38 | N | CCl₂F | 2-F-phenyl | |
| VA-39 | N | CCl₂F | 2-Cl-4-F-phenyl | |
| VA-40 | N | CCl₂F | 3,4-(OCH₃)₂-phenyl | |
| VA-41 | N | CCl₃ | 2,4-Cl₂-phenyl | |
| VA-42 | N | CCl₃ | 4-Cl-phenyl | |
| VA-43 | N | CCl₃ | 3-Cl-phenyl | |
| VA-44 | N | CCl₃ | 2-Cl-phenyl | |
| VA-45 | N | CCl₃ | 2-CH₃-phenyl | |
| VA-46 | N | CCl₃ | 4-F-phenyl | |
| VA-47 | N | CCl₃ | 3-CF₃-phenyl | |
| VA-48 | N | CCl₃ | 2-F-phenyl | |
| VA-49 | N | CCl₃ | 2-Cl-4-F-phenyl | |
| VA-50 | N | CCl₃ | 3,4-(OCH₃)₂-phenyl | |
| VA-51 | N | CH(CH₃)₂ | 2,4-Cl₂-phenyl | |
| VA-52 | N | CH(CH₃)₂ | 4-Cl-phenyl | |
| VA-53 | N | CH(CH₃)₂ | 3-Cl-phenyl | |
| VA-54 | N | CH(CH₃)₂ | 2-Cl-phenyl | |
| VA-55 | N | CH(CH₃)₂ | 2-CH₃-phenyl | |
| VA-56 | N | CH(CH₃)₂ | 4-F-phenyl | |
| VA-57 | N | CH(CH₃)₂ | 3-CF₃-phenyl | |
| VA-58 | N | CH(CH₃)₂ | 2-F-phenyl | |
| VA-59 | N | CH(CH₃)₂ | 2-Cl-4-F-phenyl | |
| VA-60 | N | CH(CH₃)₂ | 3,4-(OCH₃)₂-phenyl | |
| VA-61 | N | CH₂OCH₃ | 2,4-Cl₂-phenyl | |
| VA-62 | N | CH₂OCH₃ | 4-Cl-phenyl | |
| VA-63 | N | CH₂OCH₃ | 3-Cl-phenyl | |
| VA-64 | N | CH₂OCH₃ | 2-Cl-phenyl | |
| VA-65 | N | CH₂OCH₃ | 2-CH₃-phenyl | |
| VA-66 | N | CH₂OCH₃ | 4-F-phenyl | |
| VA-67 | N | CH₂OCH₃ | 3-CF₃-phenyl | |
| VA-68 | N | CH₂OCH₃ | 2-F-phenyl | |
| VA-69 | N | CH₂OCH₃ | 2-Cl-4-F-phenyl | |
| VA-70 | N | CH₂OCH₃ | 3,4-(OCH₃)₂-phenyl | |
| VA-71 | N | 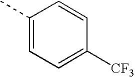 | 2,4-Cl₂-phenyl | |
| VA-72 | N | 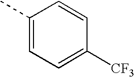 | 4-Cl-phenyl | |
| VA-73 | N | 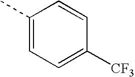 | 3-Cl-phenyl | |
| VA-74 | N | 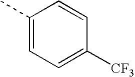 | 2-Cl-phenyl | |
| VA-75 | N | 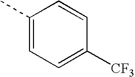 | 2-CH₃-phenyl | |
| VA-76 | N | 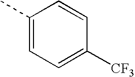 | 4-F-phenyl | |
| VA-77 | N | 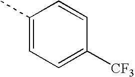 | 3-CF₃-phenyl | |
| VA-78 | N | 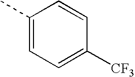 | 2-F-phenyl | |
| VA-79 | N | 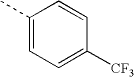 | 2-Cl-4-F-phenyl | |
| VA-80 | N | 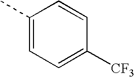 | 3,4-(OCH₃)₂-phenyl | |
| VA-81 | 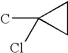 | H | 2,4-Cl₂-phenyl | |
| VA-82 | 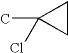 | H | 4-Cl-phenyl | |
| VA-83 | 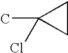 | H | 3-Cl-phenyl | |
| VA-84 | 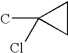 | H | 2-Cl-phenyl | |
| VA-85 | 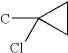 | H | 2-CH₃-phenyl | |
| VA-86 | 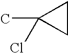 | H | 4-F-phenyl | |
| VA-87 | 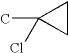 | H | 3-CF₃-phenyl | |
| VA-88 | 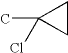 | H | 2-F-phenyl | |
| VA-89 | 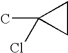 | H | 2-Cl-4-F-phenyl | |
| VA-90 | 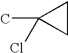 | H | 3,4-(OCH₃)₂-phenyl | |
| VA-91 | CH | CF₃ | 2,4-Cl₂-phenyl | |
| VA-92 | CH | CF₃ | 4-Cl-phenyl | |
| VA-93 | CH | CF₃ | 3-Cl-phenyl | |
| VA-94 | CH | CF₃ | 2-Cl-phenyl | |
| VA-95 | CH | CF₃ | 2-CH₃-phenyl | |
| VA-96 | CH | CF₃ | 4-F-phenyl | |
| VA-97 | CH | CF₃ | 3-CF₃-phenyl | |
| VA-98 | CH | CF₃ | 2-F-phenyl | |
| VA-99 | CH | CF₃ | 2-Cl-4-F-phenyl | |
| VA-100 | CH | CF₃ | 3,4-(OCH₃)₂-phenyl | |
| VA-101 | C—CH₃ | H | 2,4-Cl₂-phenyl | |
| VA-102 | C—CH₃ | H | 4-Cl-phenyl | |
| VA-103 | C—CH₃ | H | 3-Cl-phenyl | |
| VA-104 | C—CH₃ | H | 2-Cl-phenyl | |
| VA-105 | C—CH₃ | H | 2-CH₃-phenyl | |
| VA-106 | C—CH₃ | H | 4-F-phenyl | |
| VA-107 | C—CH₃ | H | 3-CF₃-phenyl | |
| VA-108 | C—CH₃ | H | 2-F-phenyl | |
| VA-109 | C—CH₃ | H | 2-Cl-4-F-phenyl | |
| VA-110 | C—CH₃ | H | 3,4-(OCH₃)₂-phenyl | |
| VA111 | N | CCH₃ | 2-Cl-phenyl | 2.98 |
| VA112 | N | H | 2-Cl-phenyl | 1.84 |
| VA113 | N | CF₂CF₃ | 2-Cl-phenyl | 3.19 |
| VA114 | N | CF₂CF₃ | 2-Cl-4-F-phenyl | 3.31 |
| VA115 | N | CF₃ | 2-thiazolyl | 2.48 |
| VA116 | N | CF₂CF₃ | 2-thiazolyl | 2.97 |
| VA117 | N | CF₃ | 3-Br-5-pyridyl | 2.54 |
| VA118 | N | CF₃ | 2-Cl-3-pyridyl | 2.1 |
| VA119 | N | CF₂CF₃ | 2-Cl-3-pyridyl | |
| VA120 | N | CF₃ | 4-Ph-phenyl | 3.77 |
| VA121 | N | CF₃ | 4-(4′-Cl-Ph)-phenyl | 4.26 |
| VA122 | N | CF₃ | 4-benzyloxy-phenyl | 3.73 |
| VA123 | N | CF₃ | 4-(4′-OCF₃-Ph)-phenyl | 4.46 |
| VA124 | N | CF₃ | 4-(4′-Cl-Ph)-O-phenyl | 4.22 |
| VA125 | N | CF₃ | 2-Cl-5-pyridyl | 2.42 |
| VA126 | N | CF₃ | 3-Cl-2-thienyl | 2.72 |
| VA127 | N | CF₃ | 2-MeO-phenyl | |
| VA128 | N | CF₃ | 4-CN-phenyl | |
| VA129 | N | CF₃ | 4-MeO-phenyl | |
| VA130 | N | CH₃ | 2-Cl-phenyl | |
| VA131 | N | CF₃ | 2-Br-phenyl | |
| VA133 | N | CF₃ | 2-I-phenyl | |
| VA134 | N | CF₂CF₂H | 2-Cl-4F-phenyl | 3.01 |
| VA135 | N | CF₂CF₂CF₃ | 2-Cl-4F-phenyl | 3.75 |
| VA136 | N | CF₂CF₃ | 2-Cl-4-thiazolyl | 3.25 |

TABLE 2

| | |
|---|---|
| | (IVA) |
| 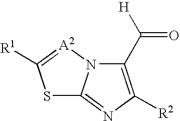 | |

| | | | | Physical |
|---|---|---|---|---|
| Ex.No. | A² | R¹ | R² | data (logP)* |
| IVA-1 | N | CF₃ | 2,4-Cl₂-phenyl | |
| IVA-2 | N | CF₃ | 4-Cl-phenyl | |
| IVA-2 | N | CF₃ | 3-Cl-phenyl | |
| IVA-3 | N | CF₃ | 2-Cl-phenyl | |
| IVA-4 | N | CF₃ | 2-CH₃-phenyl | |
| IVA-5 | N | CF₃ | 4-F-phenyl | |
| IVA-6 | N | CF₃ | 3-CF₃-phenyl | |
| IVA-7 | N | CF₃ | 2-F-phenyl | |
| IVA-8 | N | CF₃ | 2-Cl-4-F-phenyl | |
| IVA-9 | N | CF₃ | 3,4-(OCH₃)₂-phenyl | |
| IVA-10 | N | CHF₂ | 2,4-Cl₂-phenyl | |
| IVA-11 | N | CHF₂ | 4-Cl-phenyl | |
| IVA-12 | N | CHF₂ | 3-Cl-phenyl | |
| IVA-13 | N | CHF₂ | 2-Cl-phenyl | |
| IVA-14 | N | CHF₂ | 2-CH_{3-phenyl} | |
| IVA-15 | N | CHF₂ | 4-F-phenyl | |
| IVA-16 | N | CHF₂ | 3-CF₃-phenyl | |
| IVA-17 | N | CHF₂ | 2-F-phenyl | |
| IVA-18 | N | CHF₂ | 2-Cl-4-F-phenyl | |
| IVA-19 | N | CHF₂ | 3,4-(OCH₃)₂-phenyl | |
| IVA-20 | N | CClF₂ | 2,4-Cl₂-phenyl | |
| IVA-21 | N | CClF₂ | 4-Cl-phenyl | |
| IVA-22 | N | CClF₂ | 3-Cl-phenyl | |
| IVA-23 | N | CClF₂ | 2-Cl-phenyl | |
| IVA-24 | N | CClF₂ | 2-CH₃-phenyl | |
| IVA-25 | N | CClF₂ | 4-F-phenyl | |
| IVA-26 | N | CClF₂ | 3-CF₃-phenyl | |
| IVA-27 | N | CClF₂ | 2-F-phenyl | |
| IVA-28 | N | CClF₂ | 2-Cl-4-F-phenyl | |
| IVA-29 | N | CClF₂ | 3,4-(OCH₃)₂-phenyl | |
| IVA-30 | N | CCl₂F | 2,4-Cl₂-phenyl | |
| IVA-31 | N | CCl₂F | 4-Cl-phenyl | |
| IVA-32 | N | CCl₂F | 3-Cl-phenyl | |
| IVA-33 | N | CCl₂F | 2-Cl-phenyl | |
| IVA-34 | N | CCl₂F | 2-CH₃-phenyl | |
| IVA-35 | N | CCl₂F | 4-F-phenyl | |
| IVA-36 | N | CCl₂F | 3-CF₃-phenyl | |
| IVA-37 | N | CCl₂F | 2-F-phenyl | |
| IVA-38 | N | CCl₂F | 2-Cl-4-F-phenyl | |
| IVA-39 | N | CCl₂F | 3,4-(OCH₃)₂-phenyl | |
| IVA-40 | N | CCl₃ | 2,4-Cl₂-phenyl | |
| IVA-41 | N | CCl₃ | 4-Cl-phenyl | |
| IVA-42 | N | CCl₃ | 3-Cl-phenyl | |
| IVA-43 | N | CCl₃ | 2-Cl-phenyl | |
| IVA-44 | N | CCl₃ | 2-CH₃-phenyl | |
| IVA-45 | N | CCl₃ | 4-F-phenyl | |
| IVA-46 | N | CCl₃ | 3-CF₃-phenyl | |
| IVA-47 | N | CCl₃ | 2-F-phenyl | |
| IVA-48 | N | CCl₃ | 2-Cl-4-F-phenyl | |
| IVA-49 | N | CCl₃ | 3,4-(OCH₃)₂-phenyl | |
| IVA-50 | N | CH(CH₃)₂ | 2,4-Cl₂-phenyl | |
| IVA-51 | N | CH(CH₃)₂ | 4-Cl-phenyl | |
| IVA-52 | N | CH(CH₃)₂ | 3-Cl-phenyl | |
| IVA-53 | N | CH(CH₃)₂ | 2-Cl-phenyl | |
| IVA-54 | N | CH(CH₃)₂ | 2-CH₃-phenyl | |
| IVA-55 | N | CH(CH₃)₂ | 4-F-phenyl | |
| IVA-56 | N | CH(CH₃)₂ | 3-CF₃-phenyl | |
| IVA-57 | N | CH(CH₃)₂ | 2-F-phenyl | |
| IVA-58 | N | CH(CH₃)₂ | 2-Cl-4-F-phenyl | |
| IVA-59 | N | CH(CH₃)₂ | 3,4-(OCH₃)₂-phenyl | |
| IVA-60 | N | CH₂OCH₃ | 2,4-Cl₂-phenyl | |
| IVA-61 | N | CH₂OCH₃ | 4-Cl-phenyl | |
| IVA-62 | N | CH₂OCH₃ | 3-Cl-phenyl | |
| IVA-63 | N | CH₂OCH₃ | 2-Cl-phenyl | |
| IVA-64 | N | CH₂OCH₃ | 2-CH₃-phenyl | |
| IVA-65 | N | CH₂OCH₃ | 4-F-phenyl | |
| IVA-66 | N | CH₂OCH₃ | 3-CF₃-phenyl | |
| IVA-67 | N | CH₂OCH₃ | 2-F-phenyl | |
| IVA-68 | N | CH₂OCH₃ | 2-Cl-4-F-phenyl | |
| IVA-69 | N | CH₂OCH₃ | 3,4-(OCH₃)₂-phenyl | |
| IVA-70 | N | 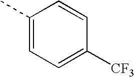 | 2,4-Cl₂-phenyl | |
| IVA-71 | N | 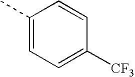 | 4-Cl-phenyl | |
| IVA-72 | N | 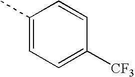 | 3-Cl-phenyl | |
| IVA-73 | N | 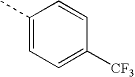 | 2-Cl-phenyl | |
| IVA-74 | N | 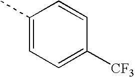 | 2-CH₃-phenyl | |
| IVA-75 | N | 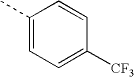 | 4-F-phenyl | |
| IVA-76 | N | 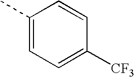 | 3-CF₃-phenyl | |
| IVA-77 | N | 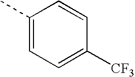 | 2-F-phenyl | |
| IVA-78 | N | 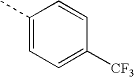 | 2-Cl-4-F-phenyl | |
| IVA-79 | N | 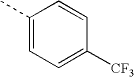 | 3,4-(OCH₃)₂-phenyl | |
| IVA-80 | N | CH₂OCH₃ | 2,4-Cl₂-phenyl | |
| IVA-81 | N | CH₂OCH₃ | 4-Cl-phenyl | |
| IVA-82 | N | CH₂OCH₃ | 3-Cl-phenyl | |
| IVA-83 | N | CH₂OCH₃ | 2-Cl-phenyl | |
| IVA-84 | N | CH₂OCH₃ | 2-CH₃-phenyl | |
| IVA-85 | N | CH₂OCH₃ | 4-F-phenyl | |
| IVA-86 | N | CH₂OCH₃ | 3-CF₃-phenyl | |
| IVA-87 | N | CH₃OCH₃ | 2-F-phenyl | |
| IVA-88 | N | CH₂OCH₃ | 2-Cl-4-F-phenyl | |
| IVA-89 | 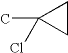 | H | 2,4-Cl₂-phenyl | |
| IVA-90 | 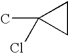 | H | 4-Cl-phenyl | |
| IVA-91 | 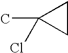 | H | 3-Cl-phenyl | |
| IVA-92 | 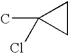 | H | 2-Cl-phenyl | |
| IVA-93 | 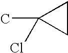 | H | 2-CH₃-phenyl | |
| IVA-94 | 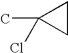 | H | 4-F-phenyl | |
| IVA-95 | 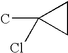 | H | 3-CF₃-phenyl | |
| IVA-96 | 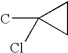 | H | 2-F-phenyl | |
| IVA-97 | 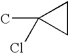 | H | 2-Cl-4-F-phenyl | |
| IVA-98 | 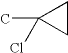 | H | 3,4-(OCH₃)₂-phenyl | |
| IVA-99 | CH | CF₃ | 2,4-Cl₂-phenyl | |
| IVA-100 | CH | CF₃ | 4-Cl-phenyl | |
| IVA-101 | CH | CF₃ | 3-Cl-phenyl | |
| IVA-102 | CH | CF₃ | 2-Cl-phenyl | |
| IVA-103 | CH | CF₃ | 2-CH₃-phenyl | |
| IVA-104 | CH | CF₃ | 4-F-phenyl | |
| IVA-105 | CH | CF₃ | 3-CF₃-phenyl | |
| IVA-106 | CH | CF₃ | 2-F-phenyl | |
| IVA-107 | CH | CF₃ | 2-Cl-4-F-phenyl | |
| IVA-108 | CH | CF₃ | 3,4-(OCH₃)₂-phenyl | |
| IVA-109 | N | CCH₃ | 2-Cl-phenyl | 3.21 |
| IVA110 | N | H | 2-Cl-phenyl | 1.79 |
| IVA111 | N | CF₂CF₃ | 2-Cl-phenyl | 3.47 |
| IVA112 | N | CF₂CF₃ | 2-Cl-4-F-phenyl | 3.63 |
| IVA113 | N | CF₃ | 2-thiazolyl | 2.43 |
| IVA114 | N | CF₂CF₃ | 2-thiazolyl | 3.03 |
| IVA115 | N | CF₃ | 3-Br-5-pyridyl | 2.62 |
| IVA116 | N | CF₃ | 2-Cl-3-pyridyl | 2.14 |
| IVA117 | N | CF₂CF₃ | 2-Cl-3-pyridyl | |
| IVA118 | N | CF₃ | 4-Ph-phenyl | 4.08 |
| IVA119 | N | CF₃ | 4-(4′-Cl-Ph)-phenyl | 4.63 |
| IVA120 | N | CF₃ | 4-benzyloxy-phenyl | 4.02 |
| IVA121 | N | CF₃ | 4-(4′-OCF₃-Ph)-phenyl | 4.78 |
| IVA122 | N | CF₃ | 4-(4′-Cl-Ph)-O-phenyl | 4.56 |
| IVA123 | N | CF₃ | 2-Cl-5-pyridyl | 2.54 |
| IVA124 | N | CF₃ | 3-Cl-2-thienyl | 2.92 |
| IVA125 | N | CF₃ | 2-MeO-phenyl | |
| IVA126 | N | CF₃ | 4-CN-phenyl | |
| IVA127 | N | CF₃ | 4-MeO-phenyl | |
| IVA128 | N | CH₃ | 2-Cl-phenyl | |
| IVA129 | N | CF₃ | 2-Br-phenyl | |
| IVA130 | N | CF₃ | 2-I-phenyl | |

*The determination of the logP values was carried out in accordance with EEC Directive 79/831 Annex V.A8 by HPLC (gradient method, acetonitrile/0.1% strength aqueous phosphoric acid or formic acid).

The compounds of the formula (IA) listed in Table 3 below are or were also prepared analogously to Preparation Example 0001 and in accordance with the general description of the preparation processes according to the invention. The hatched bonds represent the linking bond; the double bond of the acrylic unit is in each case E-configured.

TABLE 3

| | |
|---|---|
| | (IA) |
| 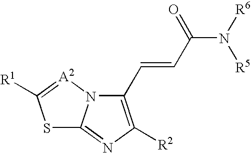 | |

| | | | | | | Physical |
|---|---|---|---|---|---|---|
| Ex. No. | A² | R¹ | R² | R⁵ | R⁶ | data (logP)* |
| 0004 | N | CF₃ | 4-Cl-phenyl | H | H | 2.62 |
| 0005 | N | CF₃ | 4-Cl-phenyl | H | CH₃ | 2.88 |
| 0006 | N | CF₃ | 4-Cl-phenyl | H | CH₂CH₃ | 3.25 |
| 0007 | N | CF₃ | 4-Cl-phenyl | H | CH(CH₃)₂ | 3.61 |
| 0008 | N | CF₃ | 4-Cl-phenyl | H | CH₂CH₂CH₃ | |
| 0009 | N | CF₃ | 4-Cl-phenyl | H | C(CH₃)₃ | 4.23 |
| 0010 | N | CF₃ | 4-Cl-phenyl | H | CH₂CH₂CH₂CH₃ | |
| 0011 | N | CF₃ | 4-Cl-phenyl | H | 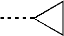 | 3.31 |
| 0012 | N | CF₃ | 4-Cl-phenyl | H | 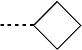 | 3.81 |
| 013 | N | CF₃ | 4-Cl-phenyl | H | 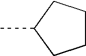 | 4.03 |
| 0014 | N | CF₃ | 4-Cl-phenyl | H | 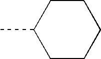 | 4.37 |
| 0015 | N | CF₃ | 4-Cl-phenyl | H | 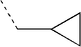 | 3.72 |
| 0016 | N | CF₃ | 4-Cl-phenyl | H | 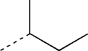 | 3.94 |
| 0017 | N | CF₃ | 4-Cl-phenyl | H | 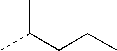 | 4.32 |
| 0018 | N | CF₃ | 4-Cl-phenyl | H | 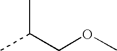 | 4.27 |
| 0019 | N | CF₃ | 4-Cl-phenyl | H | 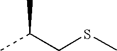 | |
| 0020 | N | CF₃ | 4-Cl-phenyl | H | 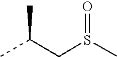 | |
| 0021 | N | CF₃ | 4-Cl-phenyl | H | 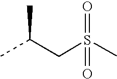 | |
| 0022 | N | CF₃ | 4-Cl-phenyl | H | 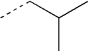 | |
| 0023 | N | CF₃ | 4-Cl-phenyl | H | 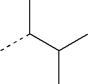 | 4.25 |
| 0024 | N | CF₃ | 4-Cl-phenyl | H | 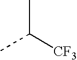 | 4.13 |
| 0025 | N | CF₃ | 4-Cl-phenyl | H | 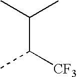 | |
| 0026 | N | CF₃ | 4-Cl-phenyl | H | 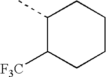 | |
| 0027 | N | CF₃ | 4-Cl-phenyl | H | 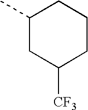 | |
| 0028 | N | CF₃ | 4-Cl-phenyl | H | 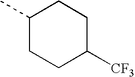 | |
| 0029 | N | CF₃ | 4-Cl-phenyl | H | 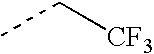 | |
| 0030 | N | CF₃ | 4-Cl-phenyl | H | 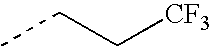 | |
| 0031 | N | CF₃ | 4-Cl-phenyl | H | 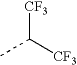 | |
| 0032 | N | CF₃ | 4-Cl-phenyl | H | 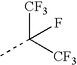 | |
| 0033 | N | CF₃ | 4-Cl-phenyl | H | 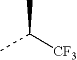 | |
| 0034 | N | CF₃ | 4-Cl-phenyl | H | 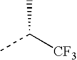 | |
| 0035 | N | CF₃ | 4-Cl-phenyl | H | 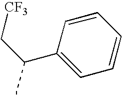 | |
| 0036 | N | CF₃ | 4-Cl-phenyl | H | 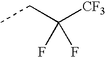 | |
| 0037 | N | CF₃ | 4-Cl-phenyl | H | 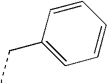 | |
| 0038 | N | CF₃ | 4-Cl-phenyl | H | 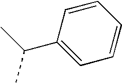 | 4.32 |
| 0039 | N | CF₃ | 4-Cl-phenyl | H | 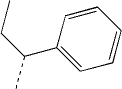 | 4.56 |
| 0040 | N | CF₃ | 4-Cl-phenyl | H | 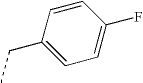 | |
| 0041 | N | CF₃ | 4-Cl-phenyl | H | 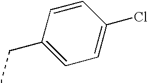 | |
| 0042 | N | CF₃ | 4-Cl-phenyl | H | 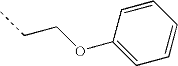 | |
| 0043 | N | CF₃ | 4-Cl-phenyl | H | 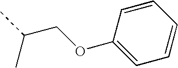 | 4.51 |
| 0044 | N | CF₃ | 4-Cl-phenyl | H | 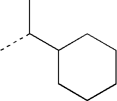 | |
| 0045 | N | CF₃ | 4-Cl-phenyl | H | 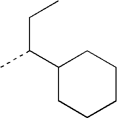 | 5.31 |
| 0046 | N | CF₃ | 4-Cl-phenyl | H | 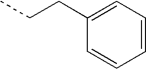 | |
| 0047 | N | CF₃ | 4-Cl-phenyl | H | 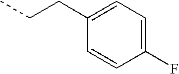 | |
| 0048 | N | CF₃ | 4-Cl-phenyl | H | 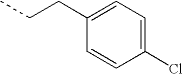 | 4.56 |
| 0049 | N | CF₃ | 4-Cl-phenyl | H | 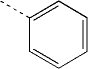 | |
| 0050 | N | CF₃ | 4-Cl-phenyl | H | 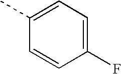 | |
| 0051 | N | CF₃ | 4-Cl-phenyl | H | 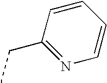 | |
| 0052 | N | CF₃ | 4-Cl-phenyl | H | 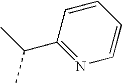 | 2.77 |
| 0053 | N | CF₃ | 4-Cl-phenyl | H | 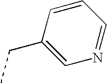 | |
| 0054 | N | CF₃ | 4-Cl-phenyl | H | 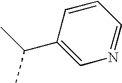 | |
| 0055 | N | CF₃ | 4-Cl-phenyl | H | 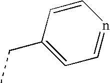 | |
| 0056 | N | CF₃ | 4-Cl-phenyl | H | 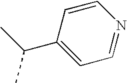 | |
| 0057 | N | CF₃ | 4-Cl-phenyl | H | OCH₃ | |
| 00058 | N | CF₃ | 4-Cl-phenyl | H | 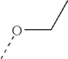 | |
| 0059 | N | CF₃ | 4-Cl-phenyl | H | 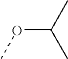 | |
| 0060 | N | CF₃ | 4-Cl-phenyl | H | 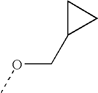 | 3.59 |
| 0061 | N | CF₃ | 4-Cl-phenyl | H | 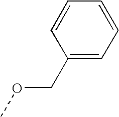 | |
| 0062 | N | CF₃ | 4-Cl-phenyl | H | 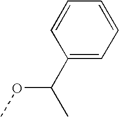 | 4.18 |
| 00063 | N | CF₃ | 4-Cl-phenyl | H | 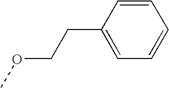 | |
| 0064 | N | CF₃ | 4-Cl-phenyl | H | 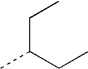 | 4.23 |
| 0065 | N | CF₃ | 4-Cl-phenyl | CH₃ | CH₃ | 3.42 |
| 0066 | N | CF₃ | 4-Cl-phenyl | CH₃ | CH₂CH₃ | |
| 0067 | N | CF₃ | 4-Cl-phenyl | CH₃ | 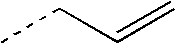 | 3.95 |
| 0068 | N | CF₃ | 4-Cl-phenyl | CH₃ | 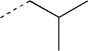 | |
| 0069 | N | CF₃ | 4-Cl-phenyl | CH₃ | OCH₃ | |
| 0070 | N | CF₃ | 4-Cl-phenyl | CH₂CH₃ | CH₂CH₃ | 4.16 |
| 0071 | N | CF₃ | 4-Cl-phenyl | 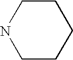 | | 4.31 |
| 0072 | N | CF₃ | 4-Cl-phenyl | 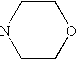 | | 3.27 |
| 0073 | N | CF₃ | 4-Cl-phenyl | 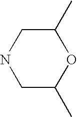 | | |
| 0074 | N | CF₃ | 4-Cl-phenyl | 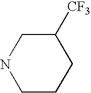 | | |
| 0075 | N | CF₃ | 3-Cl-phenyl | H | H | |
| 0076 | N | CF₃ | 3-Cl-phenyl | H | CH₃ | |
| 0077 | N | CF₃ | 3-Cl-phenyl | H | CH₂CH₃ | |
| 0078 | N | CF₃ | 3-Cl-phenyl | H | CH(CH₃)₂ | 3.67 |
| 0079 | N | CF₃ | 3-Cl-phenyl | H | CH₂CH₂CH₃ | |
| 0080 | N | CF₃ | 3-Cl-phenyl | H | C(CH₃)₃ | 4.27 |
| 0081 | N | CF₃ | 3-Cl-phenyl | H | CH₂CH₂CH₂CH₃ | |
| 0082 | N | CF₃ | 3-Cl-phenyl | H | 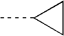 | 3.31 |
| 0083 | N | CF₃ | 3-Cl-phenyl | H | 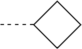 | 3.85 |
| 0084 | N | CF₃ | 3-Cl-phenyl | H | 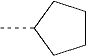 | 4.13 |
| 0085 | N | CF₃ | 3-Cl-phenyl | H | 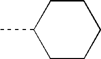 | 4.46 |
| 0086 | N | CF₃ | 3-Cl-phenyl | H | 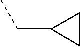 | 3.81 |
| 0087 | N | CF₃ | 3-Cl-phenyl | H | 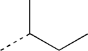 | 3.99 |
| 0088 | N | CF₃ | 3-Cl-phenyl | H | 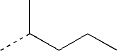 | 4.37 |
| 0089 | N | CF₃ | 3-Cl-phenyl | H | 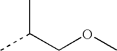 | 3.46 |
| 0090 | N | CF₃ | 3-Cl-phenyl | H | 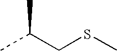 | |
| 0091 | N | CF₃ | 3-Cl-phenyl | H | 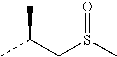 | |
| 0092 | N | CF₃ | 3-Cl-phenyl | H | 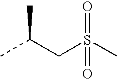 | |
| 0093 | N | CF₃ | 3-Cl-phenyl | H | 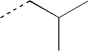 | |
| 0094 | N | CF₃ | 3-Cl-phenyl | H | 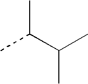 | 4.32 |
| 0095 | N | CF₃ | 3-Cl-phenyl | H | 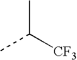 | 4.18 |
| 0095 | N | CF₃ | 3-Cl-phenyl | H | 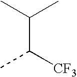 | |
| 0096 | N | CF₃ | 3-Cl-phenyl | H | 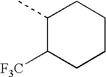 | |
| 0097 | N | CF₃ | 3-Cl-phenyl | H | 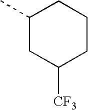 | |
| 0098 | N | CF₃ | 3-Cl-phenyl | H | 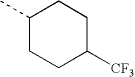 | |
| 0099 | N | CF₃ | 3-Cl-phenyl | H | 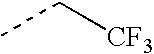 | |
| 0100 | N | CF₃ | 3-Cl-phenyl | H | 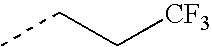 | |
| 0101 | N | CF₃ | 3-Cl-phenyl | H | 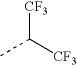 | |
| 0102 | N | CF₃ | 3-Cl-phenyl | H | 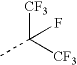 | |
| 0103 | N | CF₃ | 3-Cl-phenyl | H | 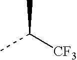 | |
| 0104 | N | CF₃ | 3-Cl-phenyl | H | 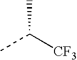 | |
| 0105 | N | CF₃ | 3-Cl-phenyl | H | 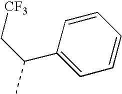 | |
| 0106 | N | CF₃ | 3-Cl-phenyl | H | 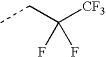 | |
| 0107 | N | CF₃ | 3-Cl-phenyl | H | 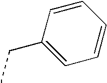 | |
| 0108 | N | CF₃ | 3-Cl-phenyl | H | 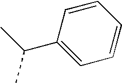 | 4.37 |
| 0109 | N | CF₃ | 3-Cl-phenyl | H | 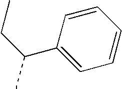 | 4.61 |
| 0110 | N | CF₃ | 3-Cl-phenyl | H | 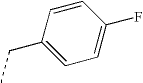 | |
| 0111 | N | CF₃ | 3-Cl-phenyl | H | 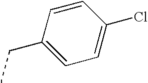 | |
| 0112 | N | CF₃ | 3-Cl-phenyl | H | 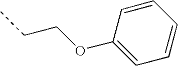 | |
| 0113 | N | CF₃ | 3-Cl-phenyl | H | 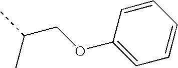 | 4.56 |
| 0114 | N | CF₃ | 3-Cl-phenyl | H | 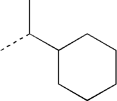 | |
| 0115 | N | CF₃ | 3-Cl-phenyl | H | 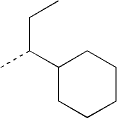 | 5.36 |
| 0116 | N | CF₃ | 3-Cl-phenyl | H | 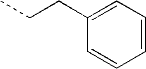 | |
| 0117 | N | CF₃ | 3-Cl-phenyl | H | 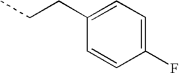 | |
| 0118 | N | CF₃ | 3-Cl-phenyl | H | 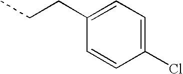 | 4.6 |
| 0119 | N | CF₃ | 3-Cl-phenyl | H | 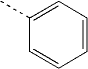 | |
| 0120 | N | CF₃ | 3-Cl-phenyl | H | 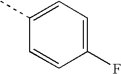 | |
| 0121 | N | CF₃ | 3-Cl-phenyl | H | 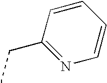 | |
| 0122 | N | CF₃ | 3-Cl-phenyl | H | 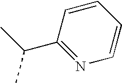 | |
| 0123 | N | CF₃ | 3-Cl-phenyl | H | 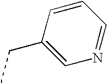 | |
| 0124 | N | CF₃ | 3-Cl-phenyl | H | 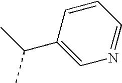 | 2.8 |
| 0125 | N | CF₃ | 3-Cl-phenyl | H | 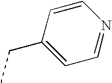 | |
| 0126 | N | CF₃ | 3-Cl-phenyl | H | 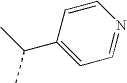 | |
| 0127 | N | CF₃ | 3-Cl-phenyl | H | OCH₃ | |
| 0128 | N | CF₃ | 3-Cl-phenyl | H | 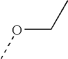 | |
| 0129 | N | CF₃ | 3-Cl-phenyl | H | 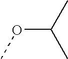 | |
| 0130 | N | CF₃ | 3-Cl-phenyl | H | 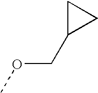 | 3.63 |
| 0131 | N | CF₃ | 3-Cl-phenyl | H | 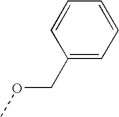 | |
| 0132 | N | CF₃ | 3-Cl-phenyl | H | 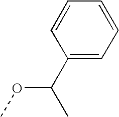 | 4.23 |
| 0133 | N | CF₃ | 3-Cl-phenyl | H | 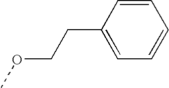 | |
| 0134 | N | CF₃ | 3-Cl-phenyl | H | 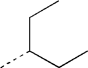 | 4.27 |
| 0135 | N | CF₃ | 3-Cl-phenyl | CH₃ | CH₃ | |
| 0136 | N | CF₃ | 3-Cl-phenyl | CH₃ | CH₂CH₃ | |
| 0137 | N | CF₃ | 3-Cl-phenyl | CH₃ | 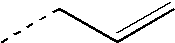 | 4.0 |
| 0138 | N | CF₃ | 3-Cl-phenyl | CH₃ | 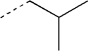 | 4.63 |
| 0139 | N | CF₃ | 3-Cl-phenyl | CH₃ | OCH₃ | |
| 0140 | N | CF₃ | 3-Cl-phenyl | CH₂CH₃ | CH₂CH₃ | 4.28 |
| 0141 | N | CF₃ | 3-Cl-phenyl | 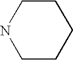 | | 4.37 |
| 0142 | N | CF₃ | 3-Cl-phenyl | 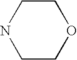 | | 3.31 |
| 0143 | N | CF₃ | 3-Cl-phenyl | 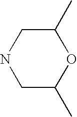 | | |
| 0144 | N | CF₃ | 3-Cl-phenyl | 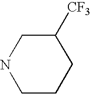 | | |
| 0145 | N | CF₃ | 2-Cl-phenyl | H | H | |
| 0146 | N | CF₃ | 2-Cl-phenyl | H | CH₃ | |
| 0147 | N | CF₃ | 2-Cl-phenyl | H | CH₂CH₃ | 2.82 |
| 0148 | N | CF₃ | 2-Cl-phenyl | H | CH(CH₃)₂ | 3.13 |
| 0149 | N | CF₃ | 2-Cl-phenyl | H | CH₂CH₂CH₃ | |
| 0150 | N | CF₃ | 2-Cl-phenyl | H | C(CH₃)₃ | 3.72 |
| 0151 | N | CF₃ | 2-Cl-phenyl | H | CH₂CH₂CH₂CH₃ | |
| 0152 | N | CF₃ | 2-Cl-phenyl | H | 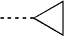 | 2.88 |
| 0153 | N | CF₃ | 2-Cl-phenyl | H | 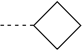 | 3.33 |
| 0154 | N | CF₃ | 2-Cl-phenyl | H | 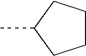 | 3.55 |
| 0155 | N | CF₃ | 2-Cl-phenyl | H | 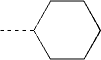 | 3.9 |
| 0166 | N | CF₃ | 2-Cl-phenyl | H | 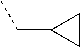 | 3.26 |
| 0167 | N | CF₃ | 2-Cl-phenyl | H | 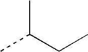 | 3.46 |
| 0168 | N | CF₃ | 2-Cl-phenyl | H | 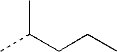 | 3.85 |
| 0169 | N | CF₃ | 2-Cl-phenyl | H | 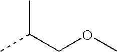 | 2.99 |
| 0170 | N | CF₃ | 2-Cl-phenyl | H | 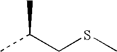 | |
| 0171 | N | CF₃ | 2-Cl-phenyl | H | 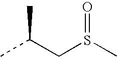 | |
| 0172 | N | CF₃ | 2-Cl-phenyl | H | 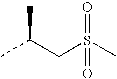 | |
| 0173 | N | CF₃ | 2-Cl-phenyl | H | 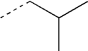 | |
| 0174 | N | CF₃ | 2-Cl-phenyl | H | 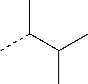 | 3.76 |
| 0175 | N | CF₃ | 2-Cl-phenyl | H | 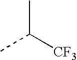 | 3.63 |
| 0176 | N | CF₃ | 2-Cl-phenyl | H | 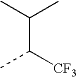 | |
| 0177 | N | CF₃ | 2-Cl-phenyl | H | 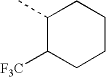 | |
| 0178 | N | CF₃ | 2-Cl-phenyl | H | 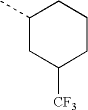 | |
| 0179 | N | CF₃ | 2-Cl-phenyl | H | 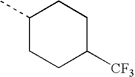 | |
| 0180 | N | CF₃ | 2-Cl-phenyl | H | 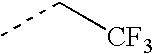 | |
| 0181 | N | CF₃ | 2-Cl-phenyl | H | 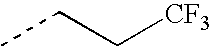 | |
| 0182 | N | CF₃ | 2-Cl-phenyl | H | 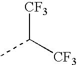 | |
| 0183 | N | CF₃ | 2-Cl-phenyl | H | 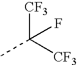 | |
| 0184 | N | CF₃ | 2-Cl-phenyl | H | 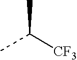 | 3.63 |
| 0185 | N | CF₃ | 2-Cl-phenyl | H | 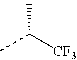 | 3.77 |
| 0186 | N | CF₃ | 2-Cl-phenyl | H | 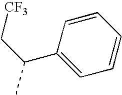 | |
| 0187 | N | CF₃ | 2-Cl-phenyl | H | 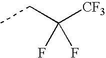 | |
| 0188 | N | CF₃ | 2-Cl-phenyl | H | 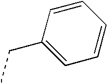 | |
| 0189 | N | CF₃ | 2-Cl-phenyl | H | 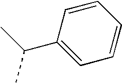 | 3.81 |
| 0190 | N | CF₃ | 2-Cl-phenyl | H | 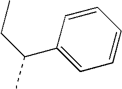 | 4.08 |
| 0191 | N | CF₃ | 2-Cl-phenyl | H | 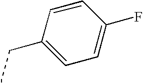 | 3.7 |
| 0192 | N | CF₃ | 2-Cl-phenyl | H | 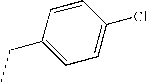 | |
| 0193 | N | CF₃ | 2-Cl-phenyl | H | 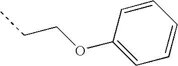 | |
| 0194 | N | CF₃ | 2-Cl-phenyl | H | 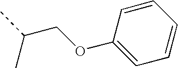 | 4.03 |
| 0195 | N | CF₃ | 2-Cl-phenyl | H | 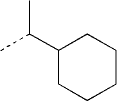 | |
| 0196 | N | CF₃ | 2-Cl-phenyl | H | 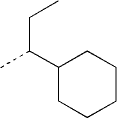 | 4.82 |
| 0197 | N | CF₃ | 2-Cl-phenyl | H | 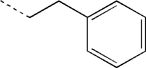 | |
| 0198 | N | CF₃ | 2-Cl-phenyl | H | 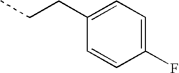 | |
| 0199 | N | CF₃ | 2-Cl-phenyl | H | 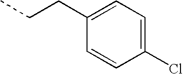 | 4.09 |
| 0200 | N | CF₃ | 2-Cl-phenyl | H | 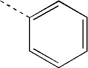 | |
| 0201 | N | CF₃ | 2-Cl-phenyl | H | 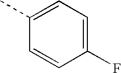 | 4.08 |
| 0202 | N | CF₃ | 2-Cl-phenyl | H | 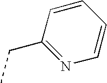 | |
| 0203 | N | CF₃ | 2-Cl-phenyl | H | 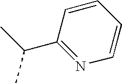 | 2.39 |
| 0204 | N | CF₃ | 2-Cl-phenyl | H | 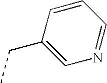 | |
| 0205 | N | CF₃ | 2-Cl-phenyl | H | 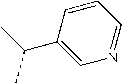 | |
| 0206 | N | CF₃ | 2-Cl-phenyl | H | 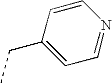 | |
| 0207 | N | CF₃ | 2-Cl-phenyl | H | 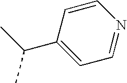 | |
| 0208 | N | CF₃ | 2-Cl-phenyl | H | OCH₃ | |
| 0209 | N | CF₃ | 2-Cl-phenyl | H | 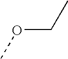 | |
| 0210 | N | CF₃ | 2-Cl-phenyl | H | 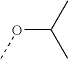 | |
| 0211 | N | CF₃ | 2-Cl-phenyl | H | 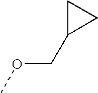 | 3.18 |
| 0212 | N | CF₃ | 2-Cl-phenyl | H | 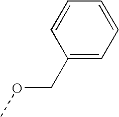 | |
| 0213 | N | CF₃ | 2-Cl-phenyl | H | 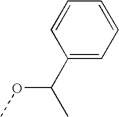 | 3.72 |
| 0214 | N | CF₃ | 2-Cl-phenyl | H | 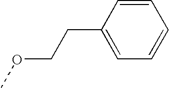 | |
| 0215 | N | CF₃ | 2-Cl-phenyl | H | 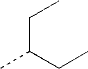 | 3.72 |
| 0216 | N | CF₃ | 2-Cl-phenyl | CH₃ | CH₃ | |
| 0217 | N | CF₃ | 2-Cl-phenyl | CH₃ | CH₂CH₃ | |
| 0218 | N | CF₃ | 2-Cl-phenyl | CH₃ | 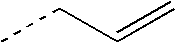 | |
| 0219 | N | CF₃ | 2-Cl-phenyl | CH₃ | 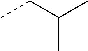 | |
| 0220 | N | CF₃ | 2-Cl-phenyl | CH₃ | OCH₃ | |
| 0221 | N | CF₃ | 2-Cl-phenyl | CH₂CH₃ | CH₂CH₃ | 3.61 |
| 0222 | N | CF₃ | 2-Cl-phenyl | 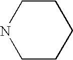 | | 3.76 |
| 0223 | N | CF₃ | 2-Cl-phenyl | 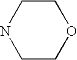 | | 2.79 |
| 0224 | N | CF₃ | 2-Cl-phenyl | 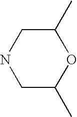 | | |
| 0225 | N | CF₃ | 2-Cl-phenyl | 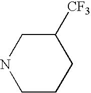 | | |
| 0226 | N | CF₃ | 2-CH₃-phenyl | H | H | |
| 0227 | N | CF₃ | 2-CH₃-phenyl | H | CH₃ | |
| 0228 | N | CF₃ | 2-CH₃-phenyl | H | CH₂CH₃ | |
| 0229 | N | CF₃ | 2-CH₃-phenyl | H | CH(CH₃)₂ | 3.24 |
| 0230 | N | CF₃ | 2-CH₃-phenyl | H | CH₂CH₂CH₃ | |
| 0231 | N | CF₃ | 2-CH₃-phenyl | H | C(CH₃)₃ | 3.85 |
| 0232 | N | CF₃ | 2-CH₃-phenyl | H | CH₂CH₂CH₂CH₃ | |
| 0233 | N | CF₃ | 2-CH₃-phenyl | H | 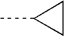 | 2.99 |
| 0234 | N | CF₃ | 2-CH₃-phenyl | H | 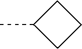 | 3.41 |
| 0235 | N | CF₃ | 2-CH₃-phenyl | H | 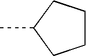 | 3.68 |
| 0236 | N | CF₃ | 2-CH₃-phenyl | H | 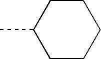 | 4.03 |
| 0237 | N | CF₃ | 2-CH₃-phenyl | H | 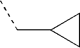 | 3.37 |
| 0238 | N | CF₃ | 2-CH₃-phenyl | H | 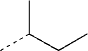 | 3.59 |
| 0239 | N | CF₃ | 2-CH₃-phenyl | H | 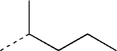 | 3.94 |
| 0240 | N | CF₃ | 2-CH₃-phenyl | H | 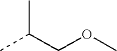 | 3.07 |
| 0241 | N | CF₃ | 2-CH₃-phenyl | H | 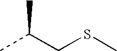 | |
| 0242 | N | CF₃ | 2-CH₃-phenyl | H | 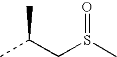 | |
| 0243 | N | CF₃ | 2-CH₃-phenyl | H | 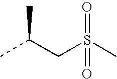 | |
| 0244 | N | CF₃ | 2-CH₃-phenyl | H | 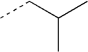 | |
| 0245 | N | CF₃ | 2-CH₃-phenyl | H | 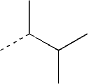 | 3.9 |
| 0246 | N | CF₃ | 2-CH₃-phenyl | H | 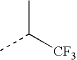 | 3.76 |
| 0247 | N | CF₃ | 2-CH₃-phenyl | H | 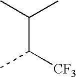 | |
| 0248 | N | CF₃ | 2-CH₃-phenyl | H | 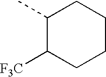 | |
| 0249 | N | CF₃ | 2-CH₃-phenyl | H | 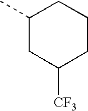 | |
| 0250 | N | CF₃ | 2-CH₃-phenyl | H | 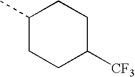 | |
| 0251 | N | CF₃ | 2-CH₃-phenyl | H | 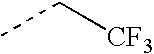 | |
| 0252 | N | CF₃ | 2-CH₃-phenyl | H | 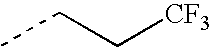 | |
| 0253 | N | CF₃ | 2-CH₃-phenyl | H | 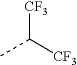 | |
| 0254 | N | CF₃ | 2-CH₃-phenyl | H | 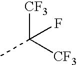 | |
| 0255 | N | CF₃ | 2-CH₃-phenyl | H | 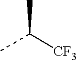 | |
| 0256 | N | CF₃ | 2-CH₃-phenyl | H | 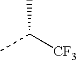 | |
| 0257 | N | CF₃ | 2-CH₃-phenyl | H | 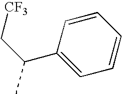 | |
| 0258 | N | CF₃ | 2-CH₃-phenyl | H | 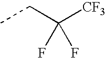 | |
| 0259 | N | CF₃ | 2-CH₃-phenyl | H | 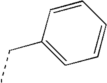 | |
| 0260 | N | CF₃ | 2-CH₃-phenyl | H | 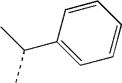 | 3.94 |
| 0261 | N | CF₃ | 2-CH₃-phenyl | H | 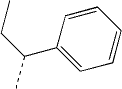 | 4.18 |
| 0262 | N | CF₃ | 2-CH₃-phenyl | H | 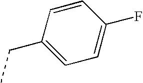 | |
| 0263 | N | CF₃ | 2-CH₃-phenyl | H | 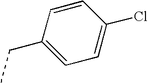 | |
| 0264 | N | CF₃ | 2-CH₃-phenyl | H | 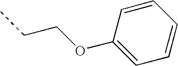 | |
| 0265 | N | CF₃ | 2-CH₃-phenyl | H | 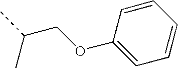 | 4.18 |
| 0266 | N | CF₃ | 2-CH₃-phenyl | H | 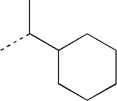 | |
| 0267 | N | CF₃ | 2-CH₃-phenyl | H | 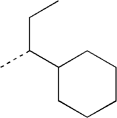 | 4.92 |
| 0268 | N | CF₃ | 2-CH₃-phenyl | H | 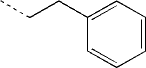 | |
| 0269 | N | CF₃ | 2-CH₃-phenyl | H | 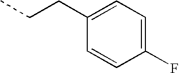 | |
| 0270 | N | CF₃ | 2-CH₃-phenyl | H | 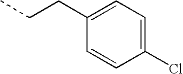 | |
| 0271 | N | CF₃ | 2-CH₃-phenyl | H | 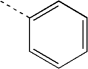 | |
| 0272 | N | CF₃ | 2-CH₃-phenyl | H | 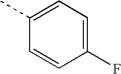 | |
| 0273 | N | CF₃ | 2-CH₃-phenyl | H | 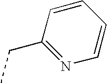 | |
| 0274 | N | CF₃ | 2-CH₃-phenyl | H | 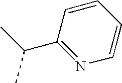 | 2.45 |
| 0275 | N | CF₃ | 2-CH₃-phenyl | H | 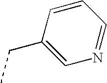 | |
| 0276 | N | CF₃ | 2-CH₃-phenyl | H | 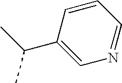 | |
| 0277 | N | CF₃ | 2-CH₃-phenyl | H | 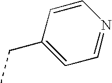 | |
| 0278 | N | CF₃ | 2-CH₃-phenyl | H | 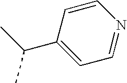 | |
| 0279 | N | CF₃ | 2-CH₃-phenyl | H | OCH₃ | |
| 0280 | N | CF₃ | 2-CH₃-phenyl | H | 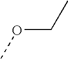 | |
| 0281 | N | CF₃ | 2-CH₃-phenyl | H | 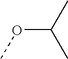 | |
| 0282 | N | CF₃ | 2-CH₃-phenyl | H | 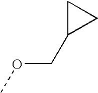 | 3.26 |
| 0283 | N | CF₃ | 2-CH₃-phenyl | H | 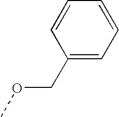 | |
| 0284 | N | CF₃ | 2-CH₃-phenyl | H | 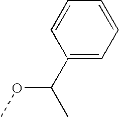 | 3.81 |
| 0285 | N | CF₃ | 2-CH₃-phenyl | H | 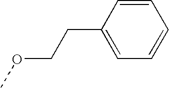 | |
| 0286 | N | CF₃ | 2-CH₃-phenyl | H | 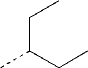 | 3.85 |
| 0287 | N | CF₃ | 2-CH₃-phenyl | CH₃ | CH₃ | |
| 0288 | N | CF₃ | 2-CH₃-phenyl | CH₃ | CH₂CH₃ | |
| 0289 | N | CF₃ | 2-CH₃-phenyl | CH₃ | 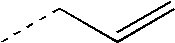 | |
| 0290 | N | CF₃ | 2-CH₃-phenyl | CH₃ | 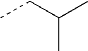 | |
| 0291 | N | CF₃ | 2-CH₃-phenyl | CH₃ | OCH₃ | |
| 0292 | N | CF₃ | 2-CH₃-phenyl | CH₂CH₃ | CH₂CH₃ | 3.75 |
| 0293 | N | CF₃ | 2-CH₃-phenyl | 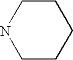 | | 3.85 |
| 0294 | N | CF₃ | 2-CH₃-phenyl | 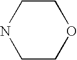 | | 2.91 |
| 0295 | N | CF₃ | 2-CH₃-phenyl | 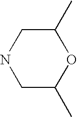 | | |
| 0296 | N | CF₃ | 2-CH₃-phenyl | 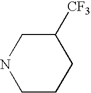 | | |
| 0297 | N | CF₃ | 4-F-phenyl | H | H | |
| 0298 | N | CF₃ | 4-F-phenyl | H | CH₃ | |
| 0299 | N | CF₃ | 4-F-phenyl | H | CH₂CH₃ | |
| 0300 | N | CF₃ | 4-F-phenyl | H | CH(CH₃)₂ | 3.24 |
| 0301 | N | CF₃ | 4-F-phenyl | H | CH₂CH₂CH₃ | |
| 0302 | N | CF₃ | 4-F-phenyl | H | C(CH₃)₃ | 3.81 |
| 0303 | N | CF₃ | 4-F-phenyl | H | CH₂CH₂CH₂CH₃ | |
| 0304 | N | CF₃ | 4-F-phenyl | H | 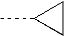 | 2.95 |
| 0305 | N | CF₃ | 4-F-phenyl | H | 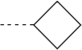 | 3.41 |
| 0306 | N | CF₃ | 4-F-phenyl | H | 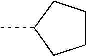 | 3.63 |
| 0307 | N | CF₃ | 4-F-phenyl | H | 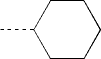 | 3.99 |
| 0308 | N | CF₃ | 4-F-phenyl | H | 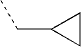 | 3.33 |
| 0309 | N | CF₃ | 4-F-phenyl | H | 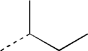 | 3.55 |
| 0310 | N | CF₃ | 4-F-phenyl | H | 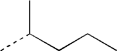 | 3.9 |
| 0311 | N | CF₃ | 4-F-phenyl | H | 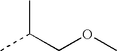 | 3.07 |
| 0312 | N | CF₃ | 4-F-phenyl | H | 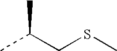 | |
| 0313 | N | CF₃ | 4-F-phenyl | H | 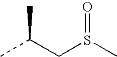 | |
| 0314 | N | CF₃ | 4-F-phenyl | H | 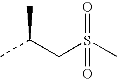 | |
| 0315 | N | CF₃ | 4-F-phenyl | H | 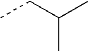 | |
| 0316 | N | CF₃ | 4-F-phenyl | H | 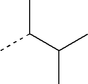 | 3.81 |
| 0317 | N | CF₃ | 4-F-phenyl | H | 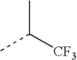 | 3.72 |
| 0318 | N | CF₃ | 4-F-phenyl | H | 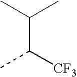 | |
| 0319 | N | CF₃ | 4-F-phenyl | H | 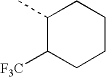 | |
| 0320 | N | CF₃ | 4-F-phenyl | H | 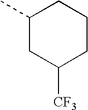 | |
| 0321 | N | CF₃ | 4-F-phenyl | H | 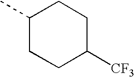 | |
| 0322 | N | CF₃ | 4-F-phenyl | H | 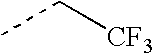 | |
| 0323 | N | CF₃ | 4-F-phenyl | H | 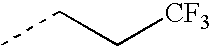 | |
| 0324 | N | CF₃ | 4-F-phenyl | H | 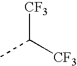 | |
| 0325 | N | CF₃ | 4-F-phenyl | H | 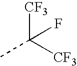 | |
| 0326 | N | CF₃ | 4-F-phenyl | H | 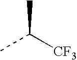 | |
| 0327 | N | CF₃ | 4-F-phenyl | H | 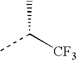 | |
| 0328 | N | CF₃ | 4-F-phenyl | H | 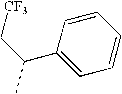 | |
| 0329 | N | CF₃ | 4-F-phenyl | H | 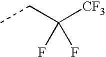 | |
| 0330 | N | CF₃ | 4-F-phenyl | H | 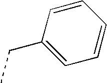 | |
| 0331 | N | CF₃ | 4-F-phenyl | H | 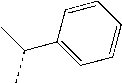 | 3.9 |
| 0332 | N | CF₃ | 4-F-phenyl | H | 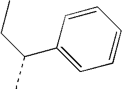 | 4.18 |
| 0333 | N | CF₃ | 4-F-phenyl | H | 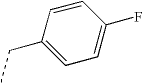 | 3.72 |
| 0334 | N | CF₃ | 4-F-phenyl | H | 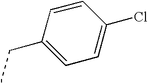 | |
| 0335 | N | CF₃ | 4-F-phenyl | H | 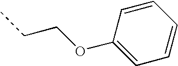 | |
| 0336 | N | CF₃ | 4-F-phenyl | H | 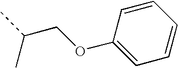 | 4.13 |
| 0337 | N | CF₃ | 4-F-phenyl | H | 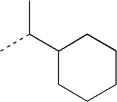 | |
| 0338 | N | CF₃ | 4-F-phenyl | H | 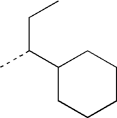 | 4.92 |
| 0339 | N | CF₃ | 4-F-phenyl | H | 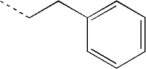 | |
| 0340 | N | CF₃ | 4-F-phenyl | H | 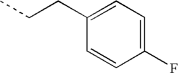 | |
| 0341 | N | CF₃ | 4-F-phenyl | H | 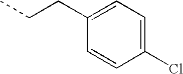 | |
| 0342 | N | CF₃ | 4-F-phenyl | H | 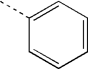 | |
| 0343 | N | CF₃ | 4-F-phenyl | H | 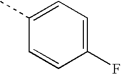 | 4.11 |
| 0344 | N | CF₃ | 4-F-phenyl | H | 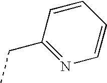 | |
| 0345 | N | CF₃ | 4-F-phenyl | H | 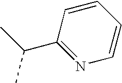 | 2.45 |
| 0346 | N | CF₃ | 4-F-phenyl | H | 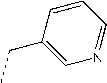 | |
| 0347 | N | CF₃ | 4-F-phenyl | H | 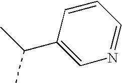 | |
| 0348 | N | CF₃ | 4-F-phenyl | H | 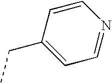 | |
| 0349 | N | CF₃ | 4-F-phenyl | H | 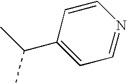 | |
| 0350 | N | CF₃ | 4-F-phenyl | H | OCH₃ | |
| 0351 | N | CF₃ | 4-F-phenyl | H | 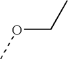 | |
| 0352 | N | CF₃ | 4-F-phenyl | H | 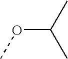 | |
| 0353 | N | CF₃ | 4-F-phenyl | H | 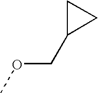 | |
| 0354 | N | CF₃ | 4-F-phenyl | H | 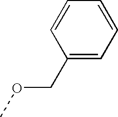 | |
| 0355 | N | CF₃ | 4-F-phenyl | H | 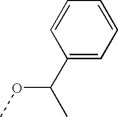 | 3.81 |
| 0356 | N | CF₃ | 4-F-phenyl | H | 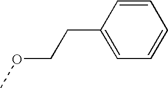 | |
| 0357 | N | CF₃ | 4-F-phenyl | H | 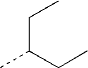 | 3.81 |
| 0358 | N | CF₃ | 4-F-phenyl | CH₃ | CH₃ | |
| 0359 | N | CF₃ | 4-F-phenyl | CH₃ | CH₂CH₃ | |
| 0360 | N | CF₃ | 4-F-phenyl | CH₃ | 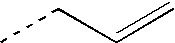 | |
| 0361 | N | CF₃ | 4-F-phenyl | CH₃ | 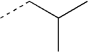 | |
| 0362 | N | CF₃ | 4-F-phenyl | CH₃ | OCH₃ | |
| 0363 | N | CF₃ | 4-F-phenyl | CH₂CH₃ | CH₂CH₃ | 3.72 |
| 0364 | N | CF₃ | 4-F-phenyl | 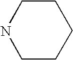 | | 3.85 |
| 0365 | N | CF₃ | 4-F-phenyl | 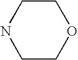 | | 2.91 |
| 0366 | N | CF₃ | 4-F-phenyl | 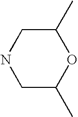 | | |
| 0367 | N | CF₃ | 4-F-phenyl | 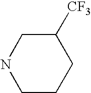 | | |
| 0368 | N | CF₃ | 3-CF₃-phenyl | H | | |
| 0369 | N | CF₃ | 3-CF₃-phenyl | H | CH₃ | |
| 0370 | N | CF₃ | 3-CF₃-phenyl | H | CH₂CH₃ | |
| 0371 | N | CF₃ | 3-CF₃-phenyl | H | CH(CH₃)₂ | 3.85 |
| 0372 | N | CF₃ | 3-CF₃-phenyl | H | CH₂CH₂CH₃ | |
| 0373 | N | CF₃ | 3-CF₃-phenyl | H | C(CH₃)₃ | 4.41 |
| 0374 | N | CF₃ | 3-CF₃-phenyl | H | CH₂CH₂CH₂CH₃ | |
| 0375 | N | CF₃ | 3-CF₃-phenyl | H | 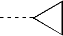 | 3.59 |
| 0376 | N | CF₃ | 3-CF₃-phenyl | H | 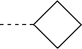 | 4.03 |
| 0377 | N | CF₃ | 3-CF₃-phenyl | H | 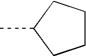 | 4.27 |
| 0378 | N | CF₃ | 3-CF₃-phenyl | H | 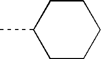 | 4.61 |
| 0379 | N | CF₃ | 3-CF₃-phenyl | H | 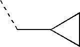 | 3.94 |
| 0380 | N | CF₃ | 3-CF₃-phenyl | H | 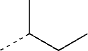 | 4.18 |
| 0381 | N | CF₃ | 3-CF₃-phenyl | H | 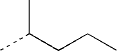 | 4.56 |
| 0382 | N | CF₃ | 3-CF₃-phenyl | H | 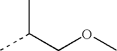 | 3.68 |
| 0383 | N | CF₃ | 3-CF₃-phenyl | H | 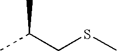 | |
| 0384 | N | CF₃ | 3-CF₃-phenyl | H | 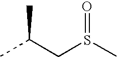 | |
| 0385 | N | CF₃ | 3-CF₃-phenyl | H | 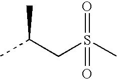 | |
| 0386 | N | CF₃ | 3-CF₃-phenyl | H | 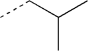 | |
| 0387 | N | CF₃ | 3-CF₃-phenyl | H | 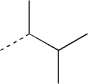 | 4.46 |
| 0388 | N | CF₃ | 3-CF₃-phenyl | H | 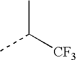 | 4.27 |
| 0389 | N | CF₃ | 3-CF₃-phenyl | H | 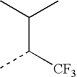 | |
| 0390 | N | CF₃ | 3-CF₃-phenyl | H | 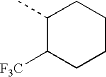 | |
| 0391 | N | CF₃ | 3-CF₃-phenyl | H | 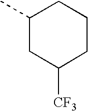 | |
| 0392 | N | CF₃ | 3-CF₃-phenyl | H | 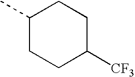 | |
| 0393 | N | CF₃ | 3-CF₃-phenyl | H | 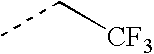 | |
| 0394 | N | CF₃ | 3-CF₃-phenyl | H | 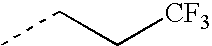 | |
| 0395 | N | CF₃ | 3-CF₃-phenyl | H | 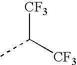 | |
| 0396 | N | CF₃ | 3-CF₃-phenyl | H | 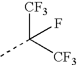 | |
| 0397 | N | CF₃ | 3-CF₃-phenyl | H | 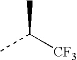 | |
| 0398 | N | CF₃ | 3-CF₃-phenyl | H | 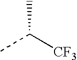 | |
| 0399 | N | CF₃ | 3-CF₃-phenyl | H | 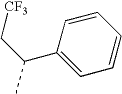 | |
| 0400 | N | CF₃ | 3-CF₃-phenyl | H | 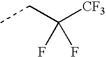 | |
| 0401 | N | CF₃ | 3-CF₃-phenyl | H | 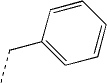 | |
| 0402 | N | CF₃ | 3-CF₃-phenyl | H | 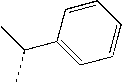 | 4.46 |
| 0403 | N | CF₃ | 3-CF₃-phenyl | H | 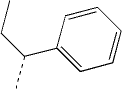 | 4.72 |
| 0404 | N | CF₃ | 3-CF₃-phenyl | H | 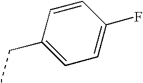 | |
| 0405 | N | CF₃ | 3-CF₃-phenyl | H | 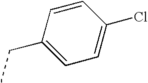 | |
| 0406 | N | CF₃ | 3-CF₃-phenyl | H | 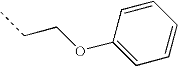 | |
| 0407 | N | CF₃ | 3-CF₃-phenyl | H | 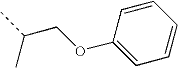 | 4.67 |
| 0408 | N | CF₃ | 3-CF₃-phenyl | H | 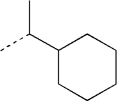 | |
| 0409 | N | CF₃ | 3-CF₃-phenyl | H | 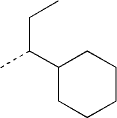 | 5.48 |
| 0410 | N | CF₃ | 3-CF_{3-phenyl} | H | 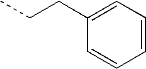 | |
| 0411 | N | CF₃ | 3-CF₃-phenyl | H | 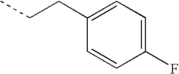 | |
| 0412 | N | CF₃ | 3-CF₃-phenyl | H | 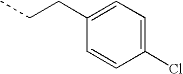 | |
| 0413 | N | CF₃ | 3-CF₃-phenyl | H | 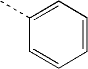 | |
| 0414 | N | CF₃ | 3-CF₃-phenyl | H | 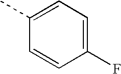 | |
| 0415 | N | CF₃ | 3-CF₃-phenyl | H | 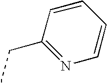 | |
| 0416 | N | CF₃ | 3-CF₃-phenyl | H | 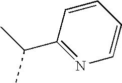 | 3.03 |
| 0417 | N | CF₃ | 3-CF₃-phenyl | H | 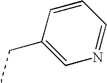 | |
| 0418 | N | CF₃ | 3-CF₃-phenyl | H | 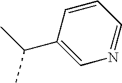 | |
| 0419 | N | CF₃ | 3-CF₃-phenyl | H | 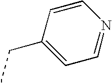 | |
| 0420 | N | CF₃ | 3-CF₃-phenyl | H | 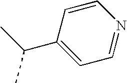 | |
| 0421 | N | CF₃ | 3-CF₃-phenyl | H | OCH₃ | |
| 0422 | N | CF₃ | 3-CF₃-phenyl | H | 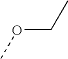 | |
| 0423 | N | CF₃ | 3-CF₃-phenyl | H | 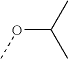 | |
| 0424 | N | CF₃ | 3-CF₃-phenyl | H | 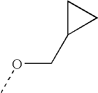 | 3.81 |
| 0425 | N | CF₃ | 3-CF₃-phenyl | H | 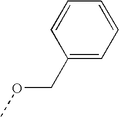 | |
| 0426 | N | CF₃ | 3-CF₃-phenyl | H | 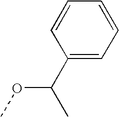 | 4.37 |
| 0427 | N | CF₃ | 3-CF₃-phenyl | H | 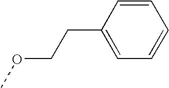 | |
| 0428 | N | CF₃ | 3-CF₃-phenyl | H | 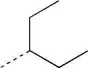 | 4.41 |
| 0429 | N | CF₃ | 3-CF₃-phenyl | CH₃ | CH₃ | 3.68 |
| 0430 | N | CF₃ | 3-CF₃-phenyl | CH₃ | CH₂CH₃ | 4.05 |
| 0431 | N | CF₃ | 3-CF₃-phenyl | CH₃ | 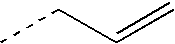 | 4.22 |
| 0432 | N | CF₃ | 3-CF₃-phenyl | CH₃ | 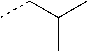 | |
| 0433 | N | CF₃ | 3-CF₃-phenyl | CH₃ | OCH₃ | 4.01 |
| 0434 | N | CF₃ | 3-CF₃-phenyl | CH₂CH₃ | CH₂CH₃ | 4.42 |
| 0435 | N | CF₃ | 3-CF₃-phenyl | 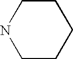 | | 4.51 |
| 0436 | N | CF₃ | 3-CF₃-phenyl | 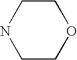 | | 3.55 |
| 0437 | N | CF₃ | 3-CF₃-phenyl | 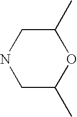 | | |
| 0438 | N | CF₃ | 3-CF₃-phenyl | 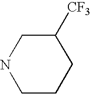 | | |
| 0439 | N | CF₃ | 2-F-phenyl | H | H | |
| 0440 | N | CF₃ | 2-F-phenyl | H | CH₃ | |
| 0441 | N | CF₃ | 2-F-phenyl | H | CH₃CH₃ | |
| 0442 | N | CF₃ | 2-F-phenyl | H | CH(CH₃)₂ | 2.96 |
| 0443 | N | CF₃ | 2-F-phenyl | H | CH₂CH₂CH₃ | |
| 0444 | N | CF₃ | 2-F-phenyl | H | C(CH₃)₃ | 3.55 |
| 0445 | N | CF₃ | 2-F-phenyl | H | CH₂CH₂CH₂CH₃ | |
| 0446 | N | CF₃ | 2-F-phenyl | H | 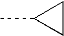 | 2.77 |
| 0447 | N | CF₃ | 2-F-phenyl | H | 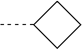 | 3.22 |
| 0448 | N | CF₃ | 2-F-phenyl | H | 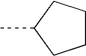 | 3.41 |
| 0449 | N | CF₃ | 2-F-phenyl | H | 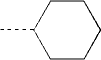 | 3.76 |
| 0450 | N | CF₃ | 2-F-phenyl | H | 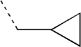 | 3.14 |
| 0451 | N | CF₃ | 2-F-phenyl | H | 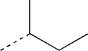 | 3.33 |
| 0452 | N | CF₃ | 2-F-phenyl | H | 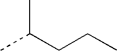 | 3.68 |
| 0453 | N | CF₃ | 2-F-phenyl | H | 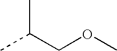 | 2.84 |
| 0454 | N | CF₃ | 2-F-phenyl | H | 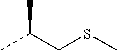 | |
| 0455 | N | CF₃ | 2-F-phenyl | H | 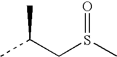 | |
| 0456 | N | CF₃ | 2-F-phenyl | H | 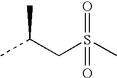 | |
| 0457 | N | CF₃ | 2-F-phenyl | H | 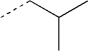 | |
| 0458 | N | CF₃ | 2-F-phenyl | H | 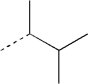 | 3.63 |
| 0459 | N | CF₃ | 2-F-phenyl | H | 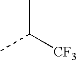 | 3.55 |
| 0460 | N | CF₃ | 2-F-phenyl | H | 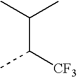 | |
| 0461 | N | CF₃ | 2-F-phenyl | H | 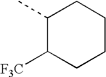 | |
| 0462 | N | CF₃ | 2-F-phenyl | H | 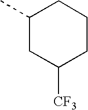 | |
| 0463 | N | CF₃ | 2-F-phenyl | H | 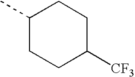 | |
| 0464 | N | CF₃ | 2-F-phenyl | H | 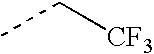 | |
| 0465 | N | CF₃ | 2-F-phenyl | H | 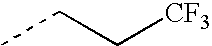 | |
| 0466 | N | CF₃ | 2-F-phenyl | H | 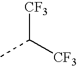 | |
| 0467 | N | CF₃ | 2-F-phenyl | H | 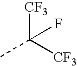 | |
| 0468 | N | CF₃ | 2-F-phenyl | H | 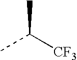 | |
| 0469 | N | CF₃ | 2-F-phenyl | H | 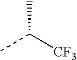 | |
| 0470 | N | CF₃ | 2-F-phenyl | H | 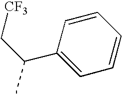 | |
| 0471 | N | CF₃ | 2-F-phenyl | H | 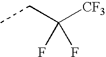 | |
| 0472 | N | CF₃ | 2-F-phenyl | H | 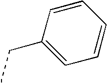 | |
| 0473 | N | CF₃ | 2-F-phenyl | H | 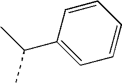 | 3.68 |
| 0474 | N | CF₃ | 2-F-phenyl | H | 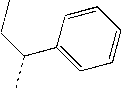 | 3.94 |
| 0475 | N | CF₃ | 2-F-phenyl | H | 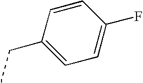 | |
| 0476 | N | CF₃ | 2-F-phenyl | H | 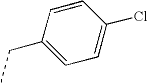 | |
| 0477 | N | CF₃ | 2-F-phenyl | H | 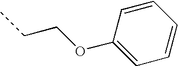 | |
| 0478 | N | CF₃ | 2-F-phenyl | H | 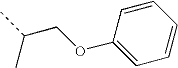 | 3.9 |
| 0479 | N | CF₃ | 2-F-phenyl | H | 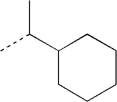 | |
| 0480 | N | CF₃ | 2-F-phenyl | H | 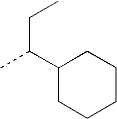 | 4.61 |
| 0481 | N | CF₃ | 2-F-phenyl | H | 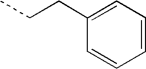 | |
| 0482 | N | CF₃ | 2-F-phenyl | H | 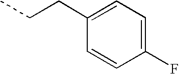 | |
| 0483 | N | CF₃ | 2-F-phenyl | H | 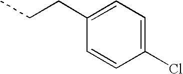 | |
| 0484 | N | CF₃ | 2-F-phenyl | H | 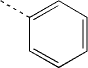 | |
| 0485 | N | CF₃ | 2-F-phenyl | H | 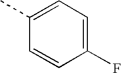 | |
| 0486 | N | CF₃ | 2-F-phenyl | H | 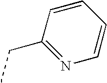 | |
| 0487 | N | CF₃ | 2-F-phenyl | H | 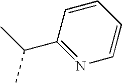 | 2.23 |
| 0488 | N | CF₃ | 2-F-phenyl | H | 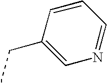 | |
| 0489 | N | CF₃ | 2-F-phenyl | H | 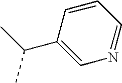 | |
| 0490 | N | CF₃ | 2-F-phenyl | H | 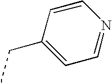 | |
| 0491 | N | CF₃ | 2-F-phenyl | H | 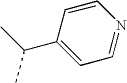 | |
| 0492 | N | CF₃ | 2-F-phenyl | H | OCH₃ | |
| 0493 | N | CF₃ | 2-F-phenyl | H | 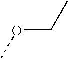 | |
| 0494 | N | CF₃ | 2-F-phenyl | H | 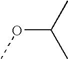 | |
| 0495 | N | CF₃ | 2-F-phenyl | H | 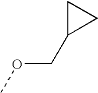 | 3.03 |
| 0496 | N | CF₃ | 2-F-phenyl | H | 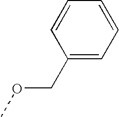 | |
| 0497 | N | CF₃ | 2-F-phenyl | H | 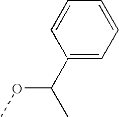 | 3.59 |
| 0498 | N | CF₃ | 2-F-phenyl | H | 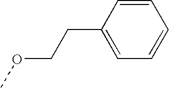 | |
| 0499 | N | CF₃ | 2-F-phenyl | H | 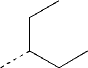 | 3.59 |
| 0500 | N | CF₃ | 2-F-phenyl | CH₃ | CH₃ | |
| 0501 | N | CF₃ | 2-F-phenyl | CH₃ | CH₂CH₃ | |
| 0502 | N | CF₃ | 2-F-phenyl | CH₃ | 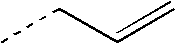 | |
| 0503 | N | CF₃ | 2-F-phenyl | CH₃ | 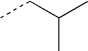 | |
| 0504 | N | CF₃ | 2-F-phenyl | CH₃ | OCH₃ | |
| 0505 | N | CF₃ | 2-F-phenyl | CH₂CH₃ | CH₂CH₃ | 3.45 |
| 0506 | N | CF₃ | 2-F-phenyl | 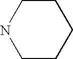 | | 3.59 |
| 0507 | N | CF₃ | 2-F-phenyl | 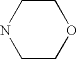 | | 2.7 |
| 0508 | N | CF₃ | 2-F-phenyl | 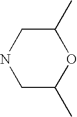 | | |
| 0509 | N | CF₃ | 2-F-phenyl | 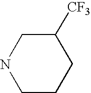 | | |
| 0510 | N | CF₃ | 2,4-Cl₂- | H | H | |
| | | | phenyl | | | |
| 0511 | N | CF₃ | 2,4-Cl₂- | H | CH₃ | |
| | | | phenyl | | | |
| 0512 | N | CF₃ | 2,4-Cl₂- | H | CH₂CH₃ | |
| | | | phenyl | | | |
| 0513 | N | CF₃ | 2,4-Cl₂- | H | CH(CH₃)₂ | 3.69 |
| | | | phenyl | | | |
| 0514 | N | CF₃ | 2,4-Cl₂- | H | CH₂CH₂CH₃ | |
| | | | phenyl | | | |
| 0515 | N | CF₃ | 2,4-Cl₂- | H | C(CH₃)₃ | 4.32 |
| | | | phenyl | | | |
| 0516 | N | CF₃ | 2,4-Cl₂- | H | CH₂CH₂CH₂CH₃ | |
| | | | phenyl | | | |
| 0517 | N | CF₃ | 2,4-Cl₂- phenyl | H | 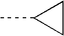 | 3.34 |
| 0518 | N | CF₃ | 2,4-Cl₂- phenyl | H | 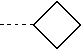 | |
| 0519 | N | CF₃ | 2,4-Cl₂- phenyl | H | 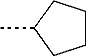 | 4.13 |
| 0520 | N | CF₃ | 2,4-Cl₂- phenyl | H | 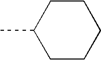 | 4.46 |
| 0521 | N | CF₃ | 2,4-Cl₂- phenyl | H | 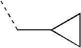 | 3.81 |
| 0522 | N | CF₃ | 2,4-Cl₂- phenyl | H | 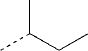 | 4.03 |
| 0523 | N | CF₃ | 2,4-Cl₂- phenyl | H | 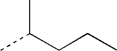 | 4.41 |
| 0524 | N | CF₃ | 2,4-Cl₂- phenyl | H | 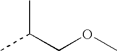 | 3.5 |
| 0525 | N | CF₃ | 2,4-Cl₂- phenyl | H | 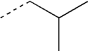 | |
| 0526 | N | CF₃ | 2,4-Cl₂- phenyl | H | 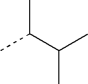 | 4.32 |
| 0527 | N | CF₃ | 2,4-Cl₂- phenyl | H | 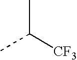 | 4.23 |
| 0528 | N | CF₃ | 2,4-Cl₂- phenyl | H | 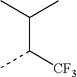 | |
| 0529 | N | CF₃ | 2,4-Cl₂- phenyl | H | 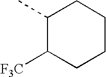 | |
| 0530 | N | CF₃ | 2,4-Cl₂- phenyl | H | 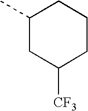 | |
| 0531 | N | CF₃ | 2,4-Cl₂- phenyl | H | 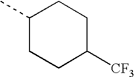 | |
| 0532 | N | CF₃ | 2,4-Cl₂- pheny | H | 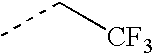 | |
| 0533 | N | CF₃ | 2,4-Cl₂- phenyl | H | 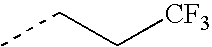 | |
| 0534 | N | CF₃ | 2,4-Cl₂- phenyl | H | 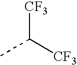 | |
| 0535 | N | CF₃ | 2,4-Cl₂- phenyl | H | 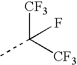 | |
| 0536 | N | CF₃ | 2,4-Cl₂- phenyl | H | 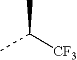 | |
| 0537 | N | CF₃ | 2,4-Cl₂- phenyl | H | 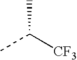 | |
| 0538 | N | CF₃ | 2,4-Cl₂- phenyl | H | 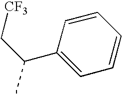 | |
| 0539 | N | CF₃ | 2,4-Cl₂- phenyl | H | 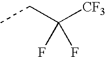 | |
| 0540 | N | CF₃ | 2,4-Cl₂- phenyl | H | 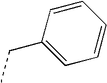 | |
| 0541 | N | CF₃ | 2,4-Cl₂- phenyl | H | 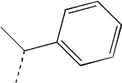 | 4.37 |
| 0542 | N | CF₃ | 2,4-Cl₂- phenyl | H | 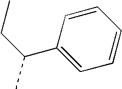 | 4.61 |
| 0543 | N | CF₃ | 2,4-Cl₂- phenyl | H | 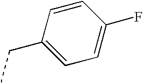 | 4.15 |
| 0544 | N | CF₃ | 2,4-Cl₂- phenyl | H | 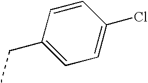 | |
| 0545 | N | CF₃ | 2,4-Cl₂- phenyl | H | 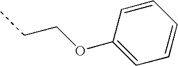 | |
| 0546 | N | CF₃ | 2,4-Cl₂- phenyl | H | 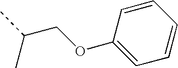 | 4.56 |
| 0547 | N | CF₃ | 2,4-Cl₂- phenyl | H | 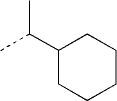 | |
| 0548 | N | CF₃ | 2,4-Cl₂- phenyl | H | 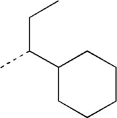 | 5.42 |
| 0549 | N | CF₃ | 2,4-Cl₂- phenyl | H | 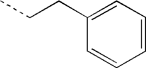 | |
| 0550 | N | CF₃ | 2,4-Cl₂- phenyl | H | 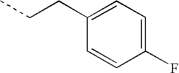 | |
| 0551 | N | CF₃ | 2,4-Cl₂- phenyl | H | 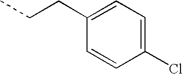 | 4.59 |
| 0552 | N | CF₃ | 2,4-Cl₂- phenyl | H | 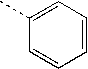 | |
| 0553 | N | CF₃ | 2,4-Cl₂- phenyl | H | 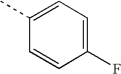 | 4.63 |
| 0554 | N | CF₃ | 2,4-Cl₂- phenyl | H | 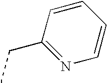 | |
| 0555 | N | CF₃ | 2,4-Cl₂- phenyl | H | 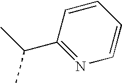 | 2.84 |
| 0556 | N | CF₃ | 2,4-Cl₂- phenyl | H | 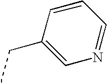 | |
| 0557 | N | CF₃ | 2,4-Cl₂- phenyl | H | 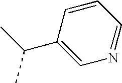 | |
| 0558 | N | CF₃ | 2,4-Cl₂- phenyl | H | 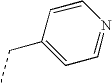 | |
| 0559 | N | CF₃ | 2,4-Cl₂- phenyl | H | 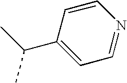 | |
| 0560 | N | CF₃ | 2,4-Cl₂- phenyl | H | OCH₃ | |
| 0561 | N | CF₃ | 2,4-Cl₂- phenyl | H | 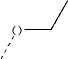 | |
| 0562 | N | CF₃ | 2,4-Cl₂- phenyl | H | 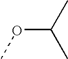 | |
| 0563 | N | CF₃ | 2,4-Cl₂- phenyl | H | 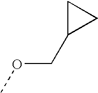 | 3.68 |
| 0564 | N | CF₃ | 2,4-Cl₂- phenyl | H | 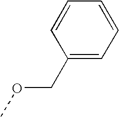 | |
| 0565 | N | CF₃ | 2,4-Cl₂- phenyl | H | 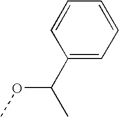 | 4.23 |
| 0566 | N | CF₃ | 2,4-Cl₂- phenyl | H | 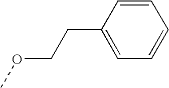 | |
| 0567 | N | CF₃ | 2,4-Cl₂- phenyl | H | 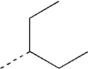 | 4.32 |
| 0568 | N | CF₃ | 2,4-Cl₂- | CH₃ | CH₃ | 3.46 |
| | | | phenyl | | | |
| 0569 | N | CF₃ | 2,4-Cl₂- | CH₃ | CH₂CH₃ | 3.82 |
| | | | phenyl | | | |
| 0570 | N | CF₃ | 2,4-Cl₂- phenyl | CH₃ | 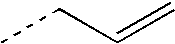 | 4.0 |
| 0571 | N | CF₃ | 2,4-Cl₂- phenyl | CH₃ | 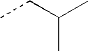 | |
| 0572 | N | CF₃ | 2,4-Cl₂- | CH₃ | OCH₃ | 3.8 |
| | | | phenyl | | | |
| 0573 | N | CF₃ | 2,4-Cl₂- | CH₂CH₃ | CH₂CH₃ | 4.18 |
| | | | phenyl | | | |
| 0574 | N | CF₃ | 2,4-Cl₂- phenyl | 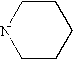 | | 4.31 |
| 0575 | N | CF₃ | 2,4-Cl₂- phenyl | 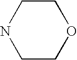 | | 3.34 |
| 0576 | N | CF₃ | 2,4-Cl₂- phenyl | 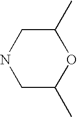 | | |
| 0577 | N | CF₃ | 2,4-Cl₂- phenyl | 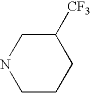 | | |
| 0578 | N | CF₃ | 2-Cl-4-F- | H | H | |
| | | | phenyl | | | |
| 0579 | N | CF₃ | 2-Cl-4-F- | H | CH₃ | |
| | | | phenyl | | | |
| 0580 | N | CF₃ | 2-Cl-4-F- | H | CH₂CH₃ | |
| | | | phenyl | | | |
| 0581 | N | CF₃ | 2-Cl-4-F- | H | CH(CH₃)₂ | 3.32 |
| | | | phenyl | | | |
| 0582 | N | CF₃ | 2-Cl-4-F- | H | CH₂CH₂CH₃ | |
| | | | phenyl | | | |
| 0583 | N | CF₃ | 2-Cl-4-F- | H | C(CH₃)₃ | 3.89 |
| | | | phenyl | | | |
| 0584 | N | CF₃ | 2-Cl-4-F- | H | CH₂CH₂CH₂CH₃ | |
| | | | phenyl | | | |
| 0585 | N | CF₃ | 2-Cl-4-F- phenyl | H | 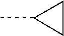 | 3.03 |
| 0586 | N | CF₃ | 2-Cl-4-F- phenyl | H | 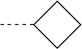 | |
| 0587 | N | CF₃ | 2-Cl-4-F- phenyl | H | 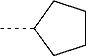 | |
| 0588 | N | CF₃ | 2-Cl-4-F- phenyl | H | 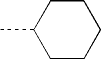 | |
| 0589 | N | CF₃ | 2-Cl-4-F- phenyl | H | 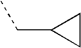 | |
| 0590 | N | CF₃ | 2-Cl-4-F- phenyl | H | 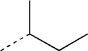 | 3.64 |
| 0591 | N | CF₃ | 2-Cl-4-F- phenyl | H | 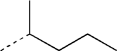 | 4.02 |
| 0592 | N | CF₃ | 2-Cl-4-F- phenyl | H | 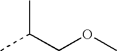 | 3.11 |
| 0593 | N | CF₃ | 2-Cl-4-F- phenyl | H | 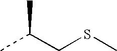 | |
| 0594 | N | CF₃ | 2-Cl-4-F- phenyl | H | 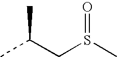 | |
| 0595 | N | CF₃ | 2-Cl-4-F- phenyl | H | 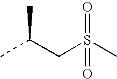 | |
| 0596 | N | CF₃ | 2-Cl-4-F- phenyl | H | 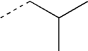 | |
| 0597 | N | CF₃ | 2-Cl-4-F- phenyl | H | 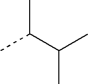 | 3.9 |
| 0598 | N | CF₃ | 2-Cl-4-F- phenyl | H | 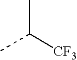 | 3.79 |
| 0599 | N | CF₃ | 2-Cl-4-F- phenyl | H | 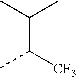 | 4.37 |
| 0600 | N | CF₃ | 2-Cl-4-F- phenyl | H | 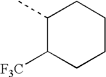 | 4.12 |
| 0601 | N | CF₃ | 2-Cl-4-F- phenyl | H | 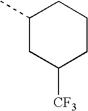 | |
| 0602 | N | CF₃ | 2-Cl-4-F- phenyl | H | 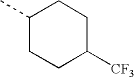 | |
| 0603 | N | CF₃ | 2-Cl-4-F- phenyl | H | 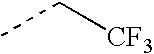 | 3.51 |
| 0604 | N | CF₃ | 2-Cl-4-F- phenyl | H | 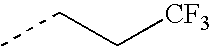 | 3.51 |
| 0605 | N | CF₃ | 2-Cl-4-F- phenyl | H | 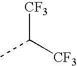 | |
| 0606 | N | CF₃ | 2-Cl-4-F- phenyl | H | 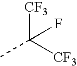 | |
| 0607 | N | CF₃ | 2-Cl-4-F- phenyl | H | 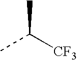 | 3.79 |
| 0608 | N | CF₃ | 2-Cl-4-F- phenyl | H | 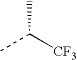 | 3.77 |
| 0609 | N | CF₃ | 2-Cl-4-F- phenyl | H | 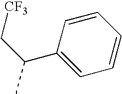 | 4.25 |
| 0610 | N | CF₃ | 2-Cl-4-F- phenyl | H | 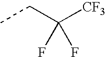 | 3.97 |
| 0611 | N | CF₃ | 2-Cl-4-F- phenyl | H | 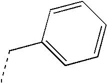 | |
| 0612 | N | CF₃ | 2-Cl-4-F- phenyl | H | 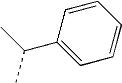 | |
| 0613 | N | CF₃ | 2-Cl-4-F- phenyl | H | 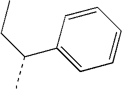 | |
| 0614 | N | CF₃ | 2-Cl-4-F- phenyl | H | 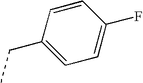 | |
| 0615 | N | CF₃ | 2-Cl-4-F- phenyl | H | 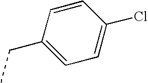 | |
| 0616 | N | CF₃ | 2-Cl-4-F- phenyl | H | 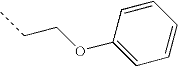 | |
| 0617 | N | CF₃ | 2-Cl-4-F- phenyl | H | 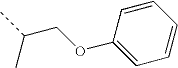 | |
| 0618 | N | CF₃ | 2-Cl-4-F- phenyl | H | 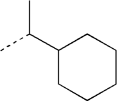 | |
| 0619 | N | CF₃ | 2-Cl-4-F- phenyl | H | 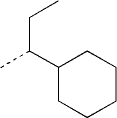 | |
| 0620 | N | CF₃ | 2-Cl-4-F- phenyl | H | 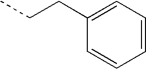 | |
| 0621 | N | CF₃ | 2-Cl-4-F- phenyl | H | 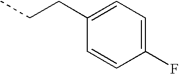 | |
| 0622 | N | CF₃ | 2-Cl-4-F- phenyl | H | 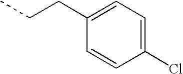 | |
| 0623 | N | CF₃ | 2-Cl-4-F- phenyl | H | 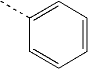 | |
| 0624 | N | CF₃ | 2-Cl-4-F- phenyl | H | 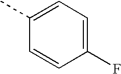 | 4.17 |
| 0625 | N | CF₃ | 2-Cl-4-F- phenyl | H | 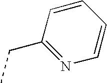 | |
| 0626 | N | CF₃ | 2-Cl-4-F- phenyl | H | 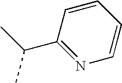 | |
| 0627 | N | CF₃ | 2-Cl-4-F- phenyl | H | 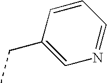 | |
| 0628 | N | CF₃ | 2-Cl-4-F- phenyl | H | 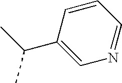 | |
| 0629 | N | CF₃ | 2-Cl-4-F- phenyl | H | 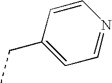 | |
| 0630 | N | CF₃ | 2-Cl-4-F- phenyl | H | 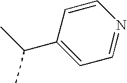 | |
| 0631 | N | CF₃ | 2-Cl-4-F- phenyl | H | OCH₃ | |
| 0632 | N | CF₃ | 2-Cl-A-F- phenyl | H | 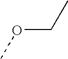 | |
| 0633 | N | CF₃ | 2-Cl-4-F- phenyl | H | 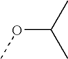 | |
| 0634 | N | CF₃ | 2-Cl-4-F- phenyl | H | 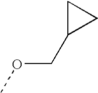 | |
| 0635 | N | CF₃ | 2-Cl-4-F- phenyl | H | 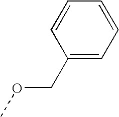 | |
| 0636 | N | CF₃ | 2-Cl-4-F- phenyl | H | 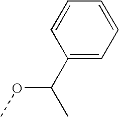 | |
| 0637 | N | CF₃ | 2-Cl-4-F- phenyl | H | 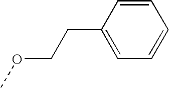 | |
| 0638 | N | CF₃ | 2-Cl-4-F- phenyl | H | 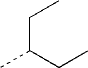 | 3.89 |
| 0639 | N | CF₃ | 2-Cl-4-F- | CH₃ | CH₃ | |
| | | | phenyl | | | |
| 0640 | N | CF₃ | 2-Cl-4-F- | CH₃ | CH₂CH₃ | |
| | | | phenyl | | | |
| 0641 | N | CF₃ | 2-Cl-4-F- phenyl | CH₃ | 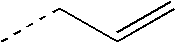 | |
| 0642 | N | CF₃ | 2-Cl-4-F- phenyl | CH₃ | 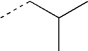 | |
| 0643 | N | CF₃ | 2-Cl-4-F- | CH₃OCH₃ | | |
| | | | phenyl | | | |
| 0644 | N | CF₃ | 2-Cl-4-F- | CH₂CH₃ | CH₂CH₃ | 3.79 |
| | | | phenyl | | | |
| 0645 | N | CF₃ | 2-Cl-4-F- phenyl | 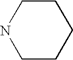 | | |
| 0646 | N | CF₃ | 2-Cl-4-F- phenyl | 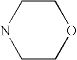 | | |
| 0647 | N | CF₃ | 2-Cl-4-F- phenyl | 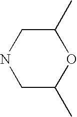 | | |
| 0648 | N | CF₃ | 2-Cl-4-F- phenyl | 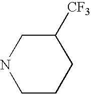 | | 4.28 |
| 0649 | N | CF₃ | 3,4-(OCH₃)₂- | H | H | |
| | | | phenyl | | | |
| 0650 | N | CF₃ | 3,4-(OCH₃)₂- | H | CH₃ | |
| | | | phenyl | | | |
| 0651 | N | CF₃ | 3,4-(OCH₃)₂- | H | CH₂CH₃ | |
| | | | phenyl | | | |
| 0652 | N | CF₃ | 3,4-(OCH₃)₂- | H | CH(CH₃)₂ | 2.85 |
| | | | phenyl | | | |
| 0653 | N | CF₃ | 3,4-(OCH₃)₂- | H | CH₂CH₂CH₃ | |
| | | | phenyl | | | |
| 0654 | N | CF₃ | 3,4-(OCH₃)₂- | H | C(CH₃)₃ | 3.37 |
| | | | phenyl | | | |
| 0655 | N | CF₃ | 3,4-(OCH₃)₃- | H | CH₂CH₂CH₂CH₃ | |
| | | | phenyl | | | |
| 0656 | N | CF₃ | 3,4-(OCH₃)₂- phenyl | H | 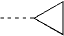 | 2.64 |
| 0657 | N | CF₃ | 3,4-(OCH₃)₂- phenyl | H | 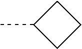 | 3.03 |
| 0658 | N | CF₃ | 3,4-(OCH₃)₂- phenyl | H | 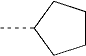 | 3.26 |
| 0659 | N | CF₃ | 3,4-(OCH₃)₂- phenyl | H | 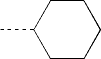 | 3.55 |
| 0660 | N | CF₃ | 3,4-(OCH₃)₂- phenyl | H | 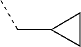 | 2.95 |
| 0661 | N | CF₃ | 3,4-(OCH₃)₂- phenyl | H | 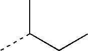 | 3.14 |
| 0662 | N | CF₃ | 3,4-(OCH₃)₂- phenyl | H | 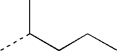 | 3.5 |
| 0663 | N | CF₃ | 3,4-(OCH₃)₂- phenyl | H | 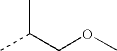 | 2.7 |
| 0664 | N | CF₃ | 3,4-(OCH₃)₂- phenyl | H | 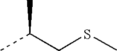 | |
| 0665 | N | CF₃ | 3,4-(OCH₃)₂- phenyl | H | 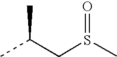 | |
| 0666 | N | CF₃ | 3,4-(OCH₃)₂- phenyl | H | 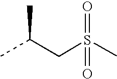 | |
| 0667 | N | CF₃ | 3,4-(OCH₃)₂- phenyl | H | 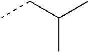 | |
| 0668 | N | CF₃ | 3,4-(OCH₃)₂- phenyl | H | 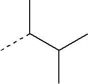 | 3.41 |
| 0669 | N | CF₃ | 3,4-(OCH₃)₂- phenyl | H | 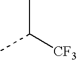 | 3.29 |
| 0670 | N | CF₃ | 3,4-(OCH₃)₂- phenyl | H | 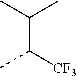 | |
| 0671 | N | CF₃ | 3,4-(OCH₃)₂- phenyl | H | 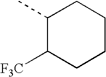 | |
| 0672 | N | CF₃ | 3,4-(OCH₃)₂- phenyl | H | 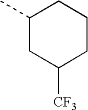 | |
| 0673 | N | CF₃ | 3,4-(OCH₃)₂- phenyl | H | 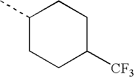 | |
| 0674 | N | CF₃ | 3,4-(OCH₃)₂- phenyl | H | 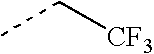 | |
| 0675 | N | CF₃ | 3,4-(OCH₃)₂- phenyl | H | 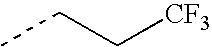 | |
| 0676 | N | CF₃ | 3,4-(OCH₃)₂- phenyl | H | 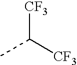 | |
| 0677 | N | CF₃ | 3,4-(OCH₃)₂- phenyl | H | 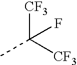 | |
| 0678 | N | CF₃ | 3,4-(OCH₃)₂- phenyl | H | 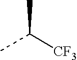 | |
| 0679 | N | CF₃ | 3,4-(OCH₃)₂- phenyl | H | 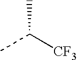 | |
| 0680 | N | CF₃ | 3,4-(OCH₃)₂- phenyl | H | 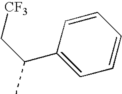 | |
| 0681 | N | CF₃ | 3,4-(OCH₃)₂- phenyl | H | 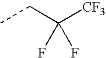 | |
| 0682 | N | CF₃ | 3,4-(OCH₃)₂- phenyl | H | 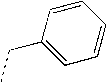 | |
| 0683 | N | CF₃ | 3,4-(OCH₃)₂- phenyl | H | 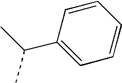 | 3.5 |
| 0684 | N | CF₃ | 3,4-(OCH₃)₂- phenyl | H | 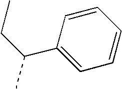 | 3.76 |
| 0685 | N | CF₃ | 3,4-(OCH₃)₂- phenyl | H | 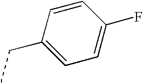 | |
| 0686 | N | CF₃ | 3,4-(OCH₃)₂- phenyl | H | 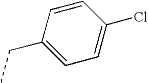 | |
| 0687 | N | CF₃ | 3,4-(OCH₃)₂- phenyl | H | 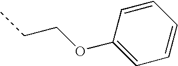 | |
| 0689 | N | CF₃ | 3,4-(OCH₃)₂- phenyl | H | 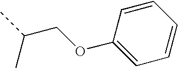 | 3.72 |
| 0690 | N | CF₃ | 3,4-(OCH₃)₂- phenyl | H | 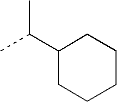 | |
| 0691 | N | CF₃ | 3,4-(OCH₃)₂- phenyl | H | 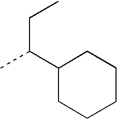 | 4.46 |
| 0692 | N | CF₃ | 3,4-(OCH₃)₂- phenyl | H | 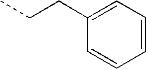 | |
| 0693 | N | CF₃ | 3,4-(OCH₃)₂- phenyl | H | 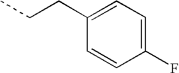 | |
| 0694 | N | CF₃ | 3,4-(OCH₃)₂- phenyl | H | 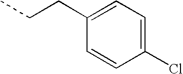 | |
| 0695 | N | CF₃ | 3,4-(OCH₃)₂- phenyl | H | 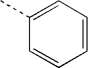 | |
| 0696 | N | CF₃ | 3,4-(OCH₃)₂- phenyl | H | 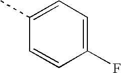 | |
| 0697 | N | CF₃ | 3,4-(OCH₃)₂- phenyl | H | 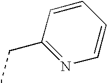 | |
| 0698 | N | CF₃ | 3,4-(OCH₃)₂- phenyl | H | 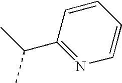 | 2.16 |
| 0699 | N | CF₃ | 3,4-(OCH₃)₂- phenyl | H | 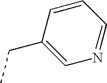 | |
| 0700 | N | CF₃ | 3,4-(OCH₃)₂- phenyl | H | 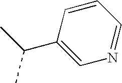 | |
| 0701 | N | CF₃ | 3,4-(OCH₃)₂- phenyl | H | 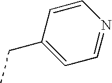 | |
| 0702 | N | CF₃ | 3,4-(OCH₃)₂- phenyl | H | 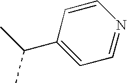 | |
| 0703 | N | CF₃ | 3,4-(OCH₃)₂- | H | OCH₃ | |
| | | | phenyl | | | |
| 0704 | N | CF₃ | 3,4-(OCH₃)₂-phenyl | H | 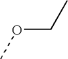 | |
| 0705 | N | CF₃ | 3,4-(OCH₃)₂-phenyl | H | 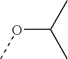 | |
| 0706 | N | CF₃ | 3,4-(OCH₃)₂- phenyl | H | 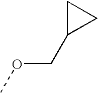 | 2.88 |
| 0707 | N | CF₃ | 3,4-(OCH₃)₂- phenyl | H | 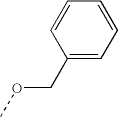 | |
| 0708 | N | CF₃ | 3,4-(OCH₃)₂- phenyl | H | 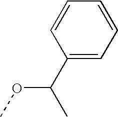 | 3.37 |
| 0709 | N | CF₃ | 3,4-(OCH₃)₂- phenyl | H | 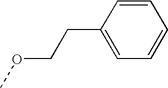 | |
| 0710 | N | CF₃ | 3,4-(OCH₃)₂- phenyl | H | 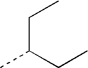 | 3.37 |
| 0711 | N | CF₃ | 3,4-(OCH₃)₂- | CH₃ | CH₃ | |
| | | | phenyl | | | |
| 0712 | N | CF₃ | 3,4-(OCH₃)₂- | CH₃ | CH₂CH₃ | |
| | | | phenyl | | | |
| 0713 | N | CF₃ | 3,4-(OCH₃)₂- phenyl | CH₃ | 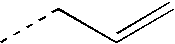 | |
| 0714 | N | CF₃ | 3,4-(OCH₃)₂- phenyl | CH₃ | 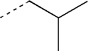 | |
| 0715 | N | CF₃ | 3,4-(OCH₃)₂- | CH₃ | OCH₃ | |
| | | | phenyl | | | |
| 0716 | N | CF₃ | 3,4-(OCH₃)₂- | CH₂CH₃ | CH₂CH₃ | 3.53 |
| | | | phenyl | | | |
| 0717 | N | CF₃ | 3,4-(OCH₃)₂- phenyl | 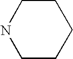 | | 3.41 |
| 0718 | N | CF₃ | 3,4-(0CH₃)₂- phenyl | 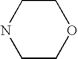 | | 2.58 |
| 0719 | N | CF₃ | 3,4-(OCH₃)₂- phenyl | 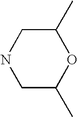 | | |
| 0720 | N | CF₃ | 3,4-(OCH₃)₂- phenyl | 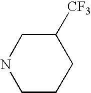 | | |
| 0721 | N | CClF₂ | 2-Cl-phenyl | H | H | |
| 0722 | N | CClF₂ | 2-Cl-phenyl | H | CH₃ | |
| 0723 | N | CClF₂ | 2-Cl-phenyl | H | CH₂CH₃ | |
| 0724 | N | CClF₂ | 2-Cl-phenyl | H | CH(CH₃)₂ | 3.36 |
| 0725 | N | CClF₂ | 2-Cl-phenyl | H | CH₂CH₂CH₃ | |
| 0726 | N | CClF₂ | 2-Cl-phenyl | H | C(CH₃)₃ | |
| 0727 | N | CClF₂ | 2-Cl-phenyl | H | CH₂CH₂CH₂CH₃ | |
| 0728 | N | CClF₂ | 2-Cl-phenyl | H | 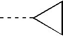 | 3.07 |
| 0729 | N | CClF₂ | 2-Cl-phenyl | H | 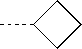 | |
| 0730 | N | CClF₂ | 2-Cl-phenyl | H | 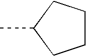 | |
| 0731 | N | CClF₂ | 2-Cl-phenyl | H | 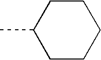 | |
| 0732 | N | CClF₂ | 2-Cl-phenyl | H | 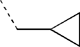 | |
| 0733 | N | CClF₂ | 2-Cl-phenyl | H | 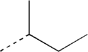 | 3.67 |
| 0734 | N | CClF₂ | 2-Cl-phenyl | H | 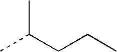 | 4.06 |
| 0735 | N | CClF₂ | 2-Cl-phenyl | H | 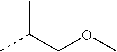 | |
| 0736 | N | CClF₂ | 2-Cl-phenyl | H | 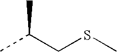 | |
| 0737 | N | CClF₂ | 2-Cl-phenyl | H | 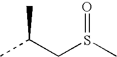 | |
| 0738 | N | CClF₂ | 2-Cl-phenyl | H | 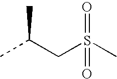 | |
| 0739 | N | CClF₂ | 2-Cl-phenyl | H | 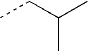 | |
| 0740 | N | CClF₂ | 2-Cl-phenyl | H | 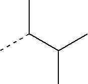 | 3.97 |
| 0741 | N | CClF₂ | 2-Cl-phenyl | H | 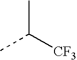 | 3.80 |
| 0742 | N | CClF₂ | 2-Cl-phenyl | H | 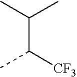 | |
| 0743 | N | CClF₂ | 2-Cl-phenyl | H | 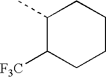 | |
| 0744 | N | CClF₂ | 2-Cl-phenyl | H | 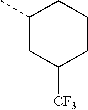 | |
| 0745 | N | CClF₂ | 2-Cl-phenyl | H | 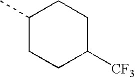 | |
| 0746 | N | CClF₂ | 2-Cl-phenyl | H | 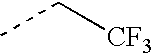 | |
| 0747 | N | CClF₂ | 2-Cl-phenyl | H | 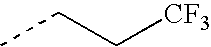 | |
| 0748 | N | CClF₂ | 2-Cl-phenyl | H | 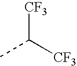 | |
| 0749 | N | CClF₂ | 2-Cl-phenyl | H | 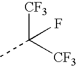 | |
| 0750 | N | CClF₂ | 2-Cl-phenyl | H | 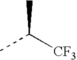 | |
| 0751 | N | CClF₂ | 2-Cl-phenyl | H | 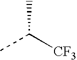 | |
| 0752 | N | CClF₂ | 2-Cl-phenyl | H | 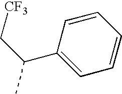 | |
| 0753 | N | CClF₂ | 2-Cl-phenyl | H | 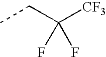 | |
| 0754 | N | CClF₂ | 2-Cl-phenyl | H | 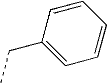 | |
| 0755 | N | CClF₂ | 2-Cl-phenyl | H | 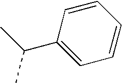 | |
| 0756 | N | CClF₂ | 2-Cl-phenyl | H | 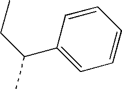 | |
| 0757 | N | CClF₂ | 2-Cl-phenyl | H | 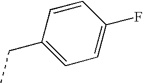 | |
| 0758 | N | CClF₂ | 2-Cl-phenyl | H | 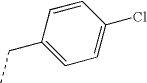 | |
| 0759 | N | CClF₂ | 2-Cl-phenyl | H | 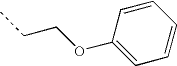 | |
| 0760 | N | CClF₂ | 2-Cl-phenyl | H | 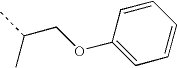 | |
| 0761 | N | CClF₂ | 2-Cl-phenyl | H | 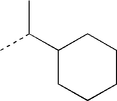 | |
| 0762 | N | CClF₂ | 2-Cl-phenyl | H | 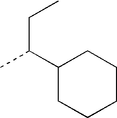 | |
| 0763 | N | CClF₂ | 2-Cl-phenyl | H | 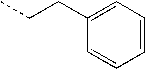 | |
| 0764 | N | CClF₂ | 2-Cl-phenyl | H | 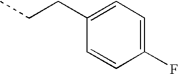 | |
| 0765 | N | CClF₂ | 2-Cl-phenyl | H | 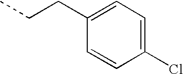 | |
| 0766 | N | CClF₂ | 2-Cl-phenyl | H | 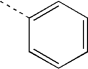 | |
| 0767 | N | CClF₂ | 2-Cl-phenyl | H | 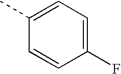 | 4.19 |
| 0768 | N | CClF₂ | 2-Cl-phenyl | H | 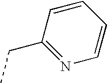 | |
| 0769 | N | CClF₂ | 2-Cl-phenyl | H | 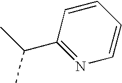 | |
| 0770 | N | CClF₂ | 2-Cl-phenyl | H | 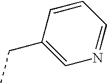 | |
| 0771 | N | CClF₂ | 2-Cl-phenyl | H | 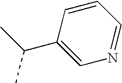 | |
| 0772 | N | CClF₂ | 2-Cl-phenyl | H | 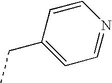 | |
| 0773 | N | CClF₂ | 2-Cl-phenyl | H | 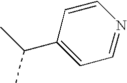 | |
| 0774 | N | CClF₂ | 2-Cl-phenyl | H | OCH₃ | |
| 0775 | N | CClF₂ | 2-Cl-phenyl | H | 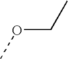 | |
| 0776 | N | CClF₂ | 2-Cl-phenyl | H | 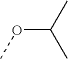 | |
| 0777 | N | CClF₂ | 2-Cl-phenyl | H | 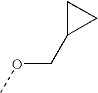 | |
| 0778 | N | CClF₂ | 2-Cl-phenyl | H | 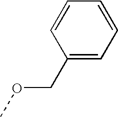 | |
| 0779 | N | CClF₂ | 2-Cl-phenyl | H | 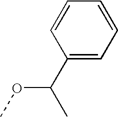 | |
| 0780 | N | CClF₂ | 2-Cl-phenyl | H | 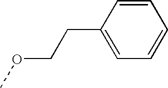 | |
| 0781 | N | CClF₂ | 2-Cl-phenyl | H | 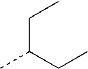 | 3.95 |
| 0782 | N | CClF₂ | 2-Cl-phenyl | CH₃ | CH₃ | |
| 0783 | N | CClF₂ | 2-Cl-phenyl | CH₃ | CH₂CH₃ | |
| 0784 | N | CClF₂ | 2-Cl-phenyl | CH₃ | 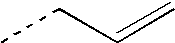 | |
| 0785 | N | CClF₂ | 2-Cl-phenyl | CH₃ | 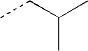 | |
| 0786 | N | CClF₂ | 2-Cl-phenyl | CH₃ | OCH₃ | |
| 0787 | N | CClF₂ | 2-Cl-phenyl | CH₂CH₃ | CH₂CH₃ | |
| 0788 | N | CClF₂ | 2-Cl-phenyl | 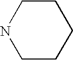 | | |
| 0789 | N | CClF₂ | 2-C-phenyl | 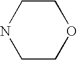 | | |
| 0790 | N | CClF₂ | 2-Cl-phenyl | 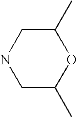 | | |
| 0791 | N | CClF₂ | 2-Cl-phenyl | 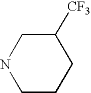 | | |
| 0792 | N | CF₃ | 2,5-Cl₂- phenyl | H | 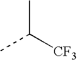 | 4.13 |
| 0793 | N | CF₃ | 3,4-Cl₂- phenyl | H | 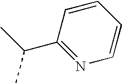 | 3.26 |
| 0794 | N | C(CH3)3 | 2-Cl-phenyl | H | 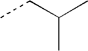 | 3.72 |
| 0795 | N | C(CH3)3 | 2-Cl-phenyl | H | 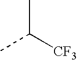 | 3.88 |
| 0796 | N | C(CH3)3 | 2-Cl-phenyl | H | 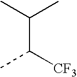 | 4.46 |
| 0797 | N | C(CH3)3 | 2-Cl-phenyl | H | 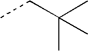 | 4.06 |
| 0798 | N | C(CH3)3 | 2-Cl-phenyl | H | 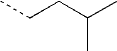 | 4.11 |
| 0799 | N | C(CH3)3 | 2-Cl-phenyl | H | 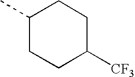 | 4.32 |
| 0800 | N | C(CH3)3 | 2-Cl-phenyl | H | 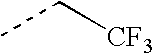 | 3.61 |
| 0801 | N | C(CH3)3 | 2-Cl-phenyl | H | 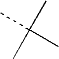 | 3.95 |
| 0802 | N | C(CH3)3 | 2-Cl-phenyl | H | 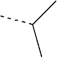 | 3.33 |
| 0803 | N | C(CH3)3 | 2-Cl-phenyl | H | 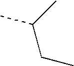 | 3.67 |
| 0804 | N | C(CH3)3 | 2-Cl-phenyl | H | 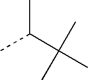 | 4.26 |
| 0805 | N | C(CH3)3 | 2-Cl-phenyl | H | 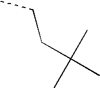 | 4.4 |
| 0806 | N | C(CH3)3 | 2-Cl-phenyl | H | 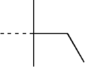 | 4.25 |
| 0807 | N | C(CH3)3 | 2-Cl-phenyl | H | 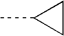 | 3.07 |
| 0808 | N | C(CH3)3 | 2-Cl-phenyl | H | 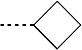 | 3.52 |
| 0809 | N | C(CH3)3 | 2-Cl-phenyl | H | 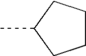 | 4.12 |
| 0810 | N | C(CH3)3 | 2-Cl-phenyl | H | 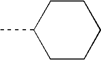 | 3.79 |
| 0811 | N | C(CH3)3 | 2-Cl-phenyl | H | 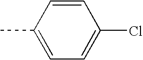 | 4.72 |
| 0812 | N | C(CH3)3 | 2-Cl-phenyl | H | 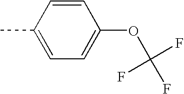 | 5.13 |
| 0813 | N | C(CH3)3 | 2-Cl-phenyl | H | 4.9 | |
| 0814 | N | C(CH3)3 | 2-Cl-phenyl | H | 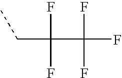 | 4.08 |
| 0815 | N | C(CH3)3 | 2-Cl-phenyl | H | 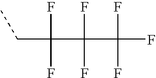 | 4.48 |
| 0816 | N | CH2—O—CH3 | 2-Cl-phenyl | H | 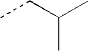 | 2.71 |
| 0817 | N | CH2—O—CH3 | 2-Cl-phenyl | H | 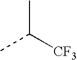 | 2.93 |
| 0818 | N | CH2—O—CH3 | 2-Cl-phenyl | H | 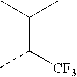 | 3.54 |
| 0819 | N | CH2—O—CH3 | 2-Cl-phenyl | H | 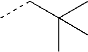 | 3.05 |
| 0820 | N | CH2—O—CH3 | 2-Cl-phenyl | H | 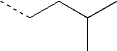 | 3.12 |
| 0821 | N | CH2—O—CH3 | 2-Cl-phenyl | H | 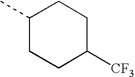 | 3.4 |
| 0822 | N | CH2—O—CH3 | 2-Cl-phenyl | H | 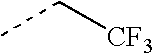 | 2.72 |
| 0823 | N | CH2—O—CH3 | 2-Cl-phenyl | H | 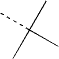 | 2.94 |
| 0824 | N | CH2—O—CH3 | 2-Cl-phenyl | H | 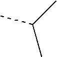 | 2.4 |
| 0825 | N | CH2—O—CH3 | 2-Cl-phenyl | H | 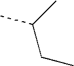 | 2.71 |
| 0826 | N | CH2—O—CH3 | 2-Cl-phenyl | H | 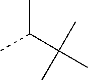 | 3.26 |
| 0827 | N | CH2—O—CH3 | 2-Cl-phenyl | H | 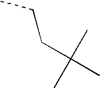 | 3.39 |
| 0828 | N | CH2—O—CH3 | 2-Cl-phenyl | H | 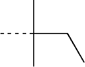 | 3.23 |
| 0829 | N | CH2—O—CH3 | 2-Cl-phenyl | H | 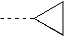 | 2.2 |
| 0830 | N | CH2—O—CH3 | 2-Cl-phenyl | H | 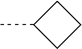 | 2.59 |
| 0831 | N | CH2—O—CH3 | 2-Cl-phenyl | H | 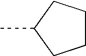 | 2.8 |
| 0832 | N | CH2—O—CH3 | 2-Cl-phenyl | H | 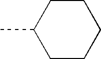 | 3.12 |
| 0833 | N | CH2—O—CH3 | 2-Cl-phenyl | H | 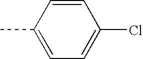 | 3.87 |
| 0834 | N | CH2—O—CH3 | 2-Cl-phenyl | H | 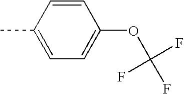 | 4 |
| 0835 | N | CH2—O—CH3 | 2-Cl-phenyl | H | 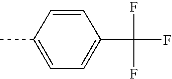 | 3.99 |
| 0836 | N | CH2—O—CH3 | 2-Cl-phenyl | H | 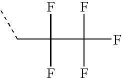 | 3.17 |
| 0837 | N | CH2—O—CH3 | 2-Cl-phenyl | H | 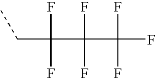 | 3.59 |
| 0838 | N | 4-CF3- phenyl | 2-Cl-phenyl | H | 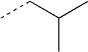 | 4.37 |
| 0839 | N | 4-CF3- phenyl | 2-Cl-phenyl | H | 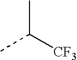 | 4.37 |
| 0840 | N | 4-CF3- phenyl | 2-Cl-phenyl | H | 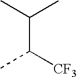 | 5 |
| 0841 | N | 4-CF3- phenyl | 2-Cl-phenyl | H | 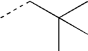 | 4.68 |
| 0842 | N | 4-CF3- phenyl | 2-Cl-phenyl | H | 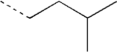 | 4.72 |
| 0843 | N | 4-CF3- phenyl | 2-Cl-phenyl | H | 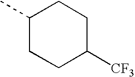 | 4.86 |
| 0844 | N | 4-CF3- phenyl | 2-Cl-phenyl | H | 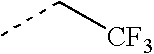 | 4.21 |
| 0845 | N | 4-CF3- phenyl | 2-Cl-phenyl | H | 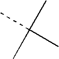 | 4.61 |
| 0846 | N | 4-CF3- phenyl | 2-Cl-phenyl | H | 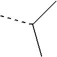 | 3.96 |
| 0847 | N | 4-CF3- phenyl | 2-Cl-phenyl | H | 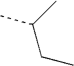 | 4.33 |
| 0848 | N | 4-CF3- phenyl | 2-Cl-phenyl | H | 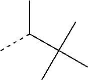 | 4.83 |
| 0849 | N | 4-CF3- phenyl | 2-Cl-phenyl | H | 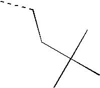 | 4.98 |
| 0850 | N | 4-CF3- phenyl | 2-Cl-phenyl | H | 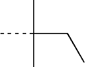 | 4.88 |
| 0851 | N | 4-CF3- phenyl | 2-Cl-phenyl | H | 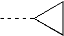 | 3.75 |
| 0852 | N | 4-CF3- phenyl | 2-Cl-phenyl | H | 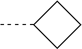 | 4.21 |
| 0853 | N | 4-CF3- phenyl | 2-Cl-phenyl | H | 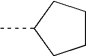 | 4.44 |
| 0854 | N | 4-CF3- phenyl | 2-Cl-phenyl | H | 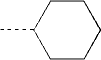 | 4.76 |
| 0855 | N | 4-CF3- phenyl | 2-Cl-phenyl | H | 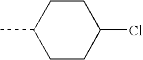 | 5.27 |
| 0856 | N | 4-CF3- phenyl | 2-Cl-phenyl | H | 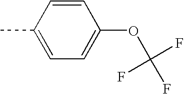 | 5.47 |
| 0857 | N | 4-CF3- phenyl | 2-Cl-phenyl | H | 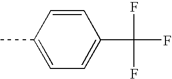 | 5.61 |
| 0858 | N | 4-CF3- phenyl | 2-Cl-phenyl | H | 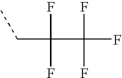 | 4.62 |
| 0859 | N | 4-CF3- phenyl | 2-Cl-phenyl | H | 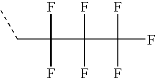 | 5 |
| 0860 | CH | CH3 | 2-Cl-phenyl | H | 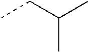 | 2.64 |
| 0861 | CH | CH3 | 2-Cl-phenyl | H | 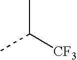 | 2.85 |
| 0862 | CH | CH3 | 2-Cl-phenyl | H | 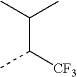 | 3.45 |
| 0863 | CH | CH3 | 2-Cl-phenyl | H | 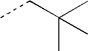 | 2.89 |
| 0864 | CH | CH3 | 2-Cl-phenyl | H | 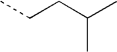 | 3 |
| 0865 | CH | CH3 | 2-Cl-phenyl | H | 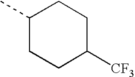 | 3.31 3.35 |
| 0866 | CH | CH3 | 2-Cl-phenyl | H | 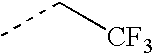 | 2.64 |
| 0867 | CH | CH3 | 2-Cl-phenyl | H | 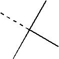 | 2.82 |
| 0868 | CH | CH3 | 2-Cl-phenyl | H | 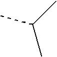 | 2.35 |
| 0869 | CH | CH3 | 2-Cl-phenyl | H | 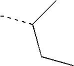 | 2.54 |
| 0870 | CH | CH3 | 2-Cl-phenyl | H | 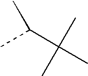 | 3.17 |
| 0871 | CH | CH3 | 2-Cl-phenyl | H | 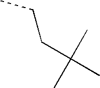 | 3.28 |
| 0872 | CH | CH3 | 2-Cl-phenyl | H | 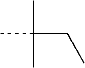 | 3.14 |
| 0873 | CH | CH3 | 2-Cl-phenyl | H | 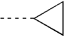 | 2.02 |
| 0874 | CH | CH3 | 2-Cl-phenyl | H | 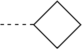 | 2.48 |
| 0875 | CH | CH3 | 2-Cl-phenyl | H | 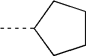 | 2.7 |
| 0876 | CH | CH3 | 2-Cl-phenyl | H | 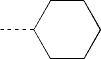 | 3.05 |
| 0877 | CH | CH3 | 2-Cl-phenyl | H | 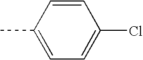 | 3.57 |
| 0878 | CH | CH3 | 2-Cl-phenyl | H | 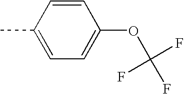 | 3.9 |
| 0879 | CH | CH3 | 2-Cl-phenyl | H | 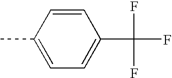 | 3.88 |
| 0880 | CH | CH3 | 2-Cl-phenyl | H | 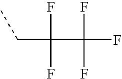 | 3.09 |
| 0881 | CH | CH3 | 2-Cl-phenyl | H | 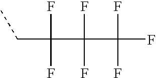 | 3.51 |
| 0882 | C—CH3 | H | 2-Cl-phenyl | H | 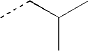 | 2.4 |
| 0883 | C—CH3 | H | 2-Cl-phenyl | H | 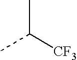 | 2.59 |
| 0884 | C—CH3 | H | 2-Cl-phenyl | H | 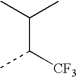 | 3.11 |
| 0885 | C—CH3 | H | 2-Cl-phenyl | H | 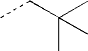 | 2.67 |
| 0886 | C—CH3 | H | 2-Cl-phenyl | H | 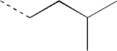 | 2.75 |
| 0887 | C—CH3 | H | 2-Cl-phenyl | H | 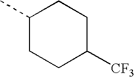 | 3.04 |
| 0888 | C—CH3 | H | 2-Cl-phenyl | H | 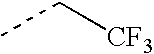 | 2.36 |
| 0889 | C—CH3 | H | 2-Cl-phenyl | H | 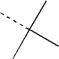 | 2.51 |
| 0890 | C—CH3 | H | 2-Cl-phenyl | H | 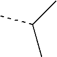 | 2.09 |
| 0891 | C—CH3 | H | 2-Cl-phenyl | H | 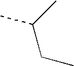 | 2.35 |
| 0892 | C—CH3 | H | 2-Cl-phenyl | H | 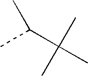 | 2.84 |
| 0893 | C—CH3 | H | 2-Cl-phenyl | H | 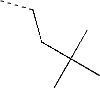 | 3.03 |
| 0894 | C—CH3 | H | 2-Cl-phenyl | H | 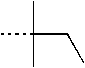 | 2.68 |
| 0895 | C—CH3 | H | 2-Cl-phenyl | H | 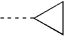 | 1.94 |
| 0896 | C—CH3 | H | 2-Cl-phenyl | H | 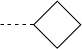 | 2.26 |
| 0897 | C—CH3 | H | 2-Cl-phenyl | H | 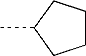 | 2.46 |
| 0898 | C—CH3 | H | 2-Cl-phenyl | H | 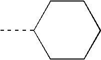 | 2.74 |
| 0899 | C—CH3 | H | 2-Cl-phenyl | H | 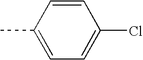 | 3.24 |
| 0900 | C—CH3 | H | 2-Cl-phenyl | H | 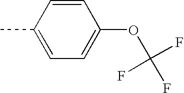 | 3.66 |
| 0901 | C—CH3 | H | 2-Cl-phenyl | H | 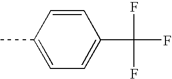 | 3.64 |
| 0902 | C—CH3 | H | 2-Cl-phenyl | H | 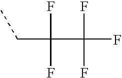 | 2.8 |
| 0903 | C—CH3 | H | 2-Cl-phenyl | H | 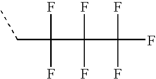 | 3.22 |
| 0904 | C-Phenyl | H | 2-Cl-phenyl | H | 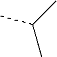 | 2.77 |
| 0905 | C-Phenyl | H | 2-Cl-phenyl | H | 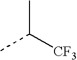 | |
| 0906 | CH | CF3 | 2-Cl-phenyl | H | 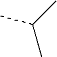 | 3.23 |
| 0907 | CH | CF3 | 2-Cl-phenyl | H | 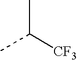 | |
| 0908 | CH | Br | 2-Cl-phenyl | H | 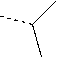 | 3.82 |
| 0909 | CH | Br | 2-Cl-phenyl | H | 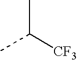 | |
| 0910 | CH(CH3)2 | H | 2-Cl-phenyl | H | 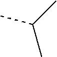 | |
| 0911 | CH(CH3)2 | H | 2-Cl-phenyl | H | 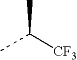 | |
| 0912 | N | CF₃ | 2-Cl-4-F- phenyl | H | 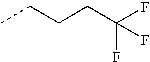 | 3.75 |
| 0913 | N | CF₃ | 2-Cl-4-F- phenyl | H | 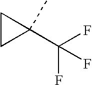 | 3.71 |
| 0914 | N | CF₃ | 2-Cl-4-F- phenyl | H | 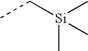 | 4.10 |
| 0915 | N | CF₃ | 2-Cl-4-F- phenyl | H | 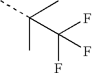 | 4.11 |
| 0916 | N | CF₃ | 2-Cl-phenyl | H | 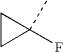 | |
| 0917 | N | CF₃ | 2-Cl-phenyl | H | 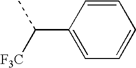 | |
| 0918 | N | CF₃ | 2-Cl-phenyl | H | 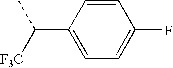 | 4.34 |
| 0919 | N | CF₃ | 2-Cl-phenyl | H | 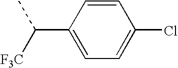 | 4.71 |
| 0920 | N | CF₃ | 3-Cl-2-thienyl | H | CH(CH₃)2 | 3.19 |
| 0921 | N | CF₃ | 3-Cl-2-thienyl | H | 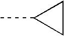 | |
| 0922 | N | CF₃ | 3-Cl-2-thienyl | H | 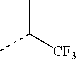 | 3.65 |
| 0923 | N | CF₃ | 3-Cl-2-thienyl | H | 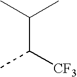 | 4.23 |
| 0924 | N | CF₃ | 3-Cl-2-thienyl | H | 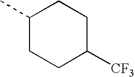 | |
| 0925 | N | CF₃ | 3-Cl-2-thienyl | H | 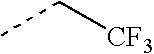 | |
| 0926 | N | CF₃ | 3-Cl-2-thienyl | H | 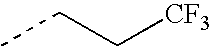 | |
| 0927 | N | CF₃ | 3-Cl-2-thienyl | H | 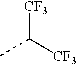 | |
| 0928 | N | CF₃ | 3-Cl-2-thienyl | H | 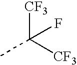 | |
| 0929 | N | CF₃ | 3-Cl-2-thienyl | H | 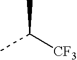 | |
| 0930 | N | CF₃ | 3-Cl-2-thienyl | H | 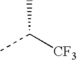 | |
| 0931 | N | CF₃ | 3-Cl-2-thienyl | H | 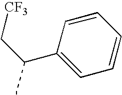 | |
| 0932 | N | CF₃ | 3-Cl-2-thienyl | H | 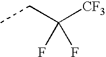 | |
| 0933 | N | CF₃ | 2-Cl-5- pyridyl | H | CH(CH₃)2 | 2.85 |
| 0934 | N | CF₃ | 2-Cl-5- pyridyl | H | 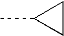 | |
| 0935 | N | CF₃ | 2-Cl-5- pyridyl | H | 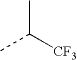 | 3.32 |
| 0936 | N | CF₃ | 2-Cl-5- pyridyl | H | 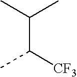 | 3.89 |
| 0937 | N | CF₃ | 2-Cl-5- pyridyl | H | 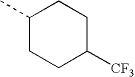 | |
| 0938 | N | CF₃ | 2-Cl-5- pyridyl | H | 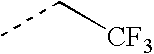 | |
| 0939 | N | CF₃ | 2-Cl-5- pyridyl | H | 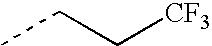 | |
| 0940 | N | CF₃ | 2-Cl-5- pyridyl | H | 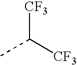 | |
| 0941 | N | CF₃ | 2-Cl-5- pyridyl | H | 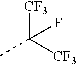 | |
| 0942 | N | CF₃ | 2-Cl-5- pyridyl | H | 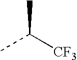 | |
| 0943 | N | CF₃ | 2-Cl-5- pyridyl | H | 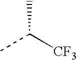 | |
| 0944 | N | CF₃ | 2-Cl-5- pyridyl | H | 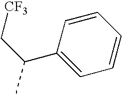 | |
| 0945 | N | CF₃ | 2-Cl-5- pyridyl | H | 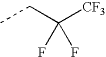 | |
| 0946 | N | CF₃ | 2-Cl-3- pyridyl | H | CH(CH₃)2 | 2.47 |
| 0947 | N | CF₃ | 2-Cl-3- pyridyl | H | 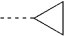 | |
| 0948 | N | CF₃ | 2-Cl-3- pyridyl | H | 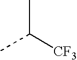 | 2.76 |
| 0949 | N | CF₃ | 2-Cl-3- pyridyl | H | 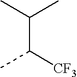 | 3.48 |
| 0950 | N | CF₃ | 2-Cl-3- pyridyl | H | 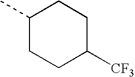 | |
| 0951 | N | CF₃ | 2-Cl-3- pyridyl | H | 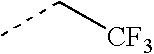 | |
| 0952 | N | CF₃ | 2-Cl-3- pyridyl | H | 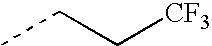 | |
| 0953 | N | CF₃ | 2-Cl-3-pyridyl | H | 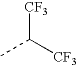 | |
| 0954 | N | CF₃ | 2-Cl-3- pyridyl | H | 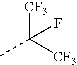 | |
| 0955 | N | CF₃ | 2-Cl-3- pyridyl | H | 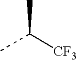 | |
| 0956 | N | CF₃ | 2-Cl-3- pyridyl | H | 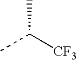 | |
| 0957 | N | CF₃ | 2-Cl-3- pyridyl | H | 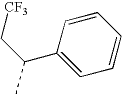 | |
| 0958 | N | CF₃ | 2-Cl-3- pyridyl | H | 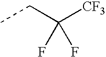 | |
| 0959 | N | CF₂CF₃ | 2-Cl-phenyl | H | CH(CH₃)2 | 3.66 |
| 0960 | N | CF₂CF₃ | 2-Cl-phenyl | H | 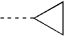 | |
| 0961 | N | CF₂CF₃ | 2-Cl-phenyl | H | 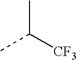 | 4.07 |
| 0962 | N | CF₂CF₃ | 2-Cl-phenyl | H | 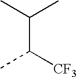 | 4.62 |
| 0963 | N | CF₂CF₃ | 2-Cl-phenyl | H | 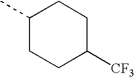 | |
| 0964 | N | CF₂CF₃ | 2-Cl-phenyl | H | 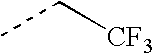 | |
| 0965 | N | CF₂CF₃ | 2-Cl-phenyl | H | 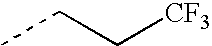 | |
| 0966 | N | CF₂CF₃ | 2-Cl-phenyl | H | 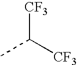 | |
| 0967 | N | CF₂CF₃ | 2-Cl-phenyl | H | 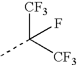 | |
| 0968 | N | CF₂CF₃ | 2-Cl-phenyl | H | 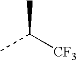 | |
| 0969 | N | CF₂CF₃ | 2-Cl-phenyl | H | 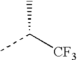 | |
| 0970 | N | CF₂CF₃ | 2-Cl-phenyl | H | 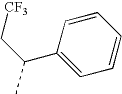 | |
| 0971 | N | CF₂CF₃ | 2-Cl-phenyl | H | 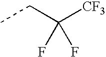 | |
| 0972 | N | CF₂CF₃ | 2-Cl-4-F- phenyl | H | CH(CH₃)2 | 3.79 |
| 0973 | N | CF₂CF₃ | 2-Cl-4-F- phenyl | H | 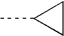 | |
| 0974 | N | CF₂CF₃ | 2-Cl-4-F- phenyl | H | 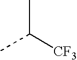 | 4.19 |
| 0975 | N | CF₂CF₃ | 2-Cl-4-F- phenyl | H | 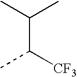 | 4.76 |
| 0976 | N | CF₂CF₃ | 2-Cl-4-F- phenyl | H | 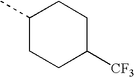 | |
| 0977 | N | CF₂CF₃ | 2-Cl-4-F- Phenyl | H | 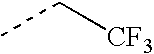 | |
| 0978 | N | CF₂CF₃ | 2-Cl-4-F- phenyl | H | 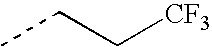 | |
| 0979 | N | CF₂CF₃ | 2-Cl-4-F- phenyl | H | 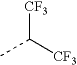 | |
| 0980 | N | CF₂CF₃ | 2-Cl-4-F- phenyl | H | 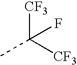 | |
| 0981 | N | CF₂CF₃ | 2-Cl-4-F- phenyl | H | 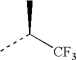 | |
| 0982 | N | CF₂CF₃ | 2-Cl-4-F- phenyl | H | 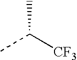 | |
| 0983 | N | CF₂CF₃ | 2-Cl-4-F- phenyl | H | 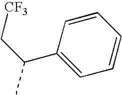 | |
| 0984 | N | CF₂CF₃ | 2-Cl-4-F- phenyl | H | 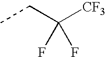 | |
| 0985 | N | CF₂CF₃ | 2-thiazolyl | H | CH(CH₃)2 | 3.27 |
| 0986 | N | CF₂CF₃ | 2-thiazolyl | H | 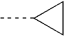 | |
| 0987 | N | CF₂CF₃ | 2-thiazolyl | H | 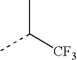 | 3.81 |
| 0988 | N | CF₂CF₃ | 2-thiazolyl | H | 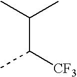 | 4.35 |
| 0989 | N | CF₂CF₃ | 2-thiazolyl | H | 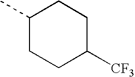 | |
| 0990 | N | CF₂CF₃ | 2-thiazolyl | H | 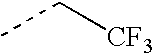 | |
| 0991 | N | CF₂CF₃ | 2-thiazolyl | H | 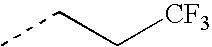 | |
| 0992 | N | CF₂CF₃ | 2-thiazolyl | H | 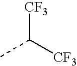 | |
| 0993 | N | CF₂CF₃ | 2-thiazolyl | H | 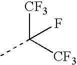 | |
| 0994 | N | CF₂CF₃ | 2-thiazolyl | H | 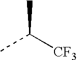 | |
| 0995 | N | CF₂CF₃ | 2-thiazolyl | H | 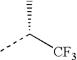 | |
| 0996 | N | CF₂CF₃ | 2-thiazolyl | H | 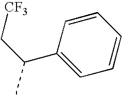 | |
| 0997 | N | CF₂CF₃ | 2-thiazolyl | H | 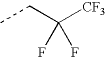 | |
| 0998 | N | CF₃ | 2-Br-phenyl | H | CH(CH₃)2 | |
| 0999 | N | CF₃ | 2-Br-phenyl | H | 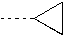 | |
| 1000 | N | CF₃ | 2-Br-phenyl | H | 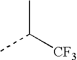 | |
| 1001 | N | CF₃ | 2-Br-phenyl | H | 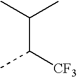 | |
| 1002 | N | CF₃ | 2-Br-phenyl | H | 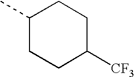 | |
| 1003 | N | CF₃ | 2-Br-phenyl | H | 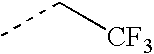 | |
| 1004 | N | CF₃ | 2-Br-phenyl | H | 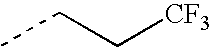 | |
| 1005 | N | CF₃ | 2-Br-phenyl | H | 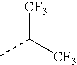 | |
| 1006 | N | CF₃ | 2-Br-phenyl | H | 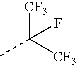 | |
| 1007 | N | CF₃ | 2-Br-phenyl | H | 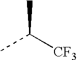 | |
| 1008 | N | CF₃ | 2-Br-phenyl | H | 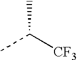 | |
| 1009 | N | CF₃ | 2-Br-phenyl | H | 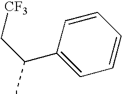 | |
| 1010 | N | CF₃ | 2-Br-phenyl | H | 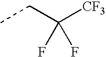 | |
| 1011 | N | CF₃ | 2-I-phenyl | H | CH(CH₃)2 | |
| 1012 | N | CF₃ | 2-I-phenyl | H | 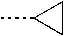 | |
| 1013 | N | CF₃ | 2-I-phenyl | H | 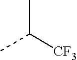 | |
| 1014 | N | CF₃ | 2-I-phenyl | H | 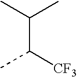 | |
| 1015 | N | CF₃ | 2-I-phenyl | H | 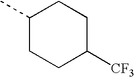 | |
| 1016 | N | CF₃ | 2-I-phenyl | H | 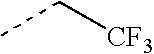 | |
| 1017 | N | CF₃ | 2-I-phenyl | H | 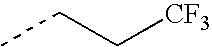 | |
| 1018 | N | CF₃ | 2-I-phenyl | H | 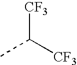 | |
| 1019 | N | CF₃ | 2-I-phenyl | H | 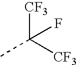 | |
| 1020 | N | CF₃ | 2-I-phenyl | H | 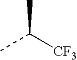 | |
| 1021 | N | CF₃ | 2-I-phenyl | H | 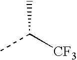 | |
| 1022 | N | CF₃ | 2-I-phenyl | H | 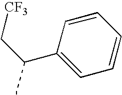 | |
| 1023 | N | CF₃ | 2-I-phenyl | H | 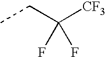 | |

*The determination of the logP values was carried out in accordance with EEC Directive 79/831 Annex V.A8 by HPLC (gradient method, acetonitrile/0.1% strength aqueous phosphoric acid or formic acid).

### Biological Data

### Insecticidal Action

### Example No. B

### Phaedon Test

### PHAECO Spray Treatment

| | | |
|---|---|---|
| Solvents: | 78 | parts by weight of acetone |
| | 1.5 | parts by weight of dimethylformamide |
| Emulsifier: | 0.5 | part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Discs of Chinese cabbage leaves (Brassica pekinensis) are sprayed with a preparation of active compound of the desired concentration and, after drying, populated with larvae of the mustard beetle (Phaedon cochleariae).

After the desired period of time, the effect in % is determined. 100% means that all beetle larvae have been killed; 0% means that none of the beetle larvae have been killed.

In this test, for example, the following compounds of the preparation examples show good activity: see Table 4.

TABLE 4

| | | PHAECO | |
|---|---|---|---|
| | | spray treatment | |
| Example | Structure | g a. i./ha | % 7 d |
| 0585 | 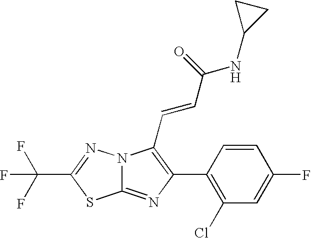 | 500 | 100 |
| 0734 | 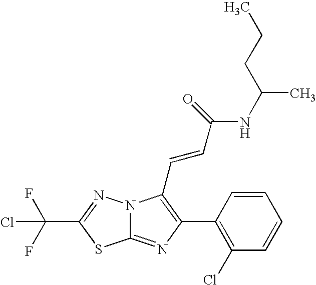 | 500 | 100 |
| 0728 | 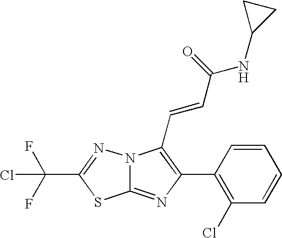 | 500 | 100 |
| 0724 | 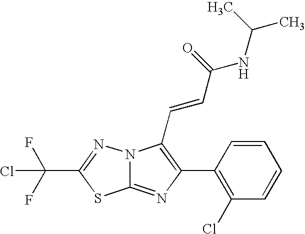 | 500 | 100 |
| 0729 | 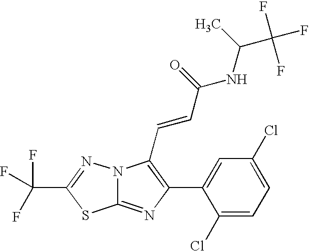 | 500 | 100 |
| 0152 | 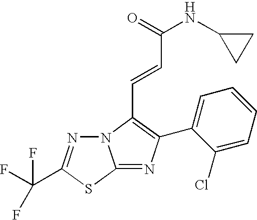 | 500 | 100 |
| 0166 | 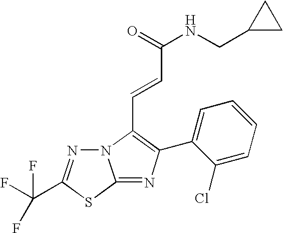 | 500 | 100 |
| 0598 | 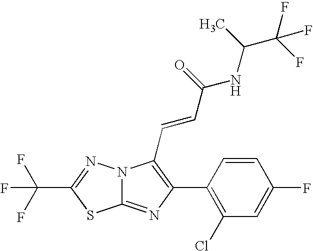 | 500 | 100 |
| 0416 | 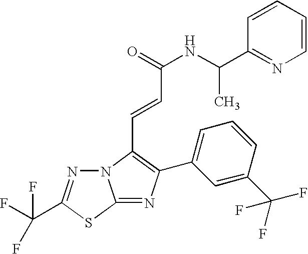 | 500 | 100 |
| 0793 | 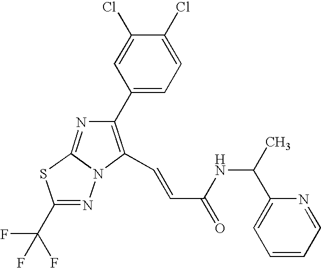 | 500 | 100 |
| 0203 | 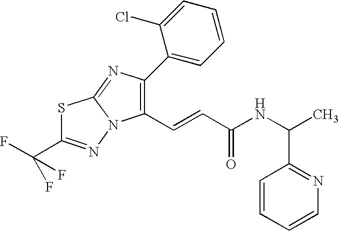 | 500 | 83 |
| 0654 | 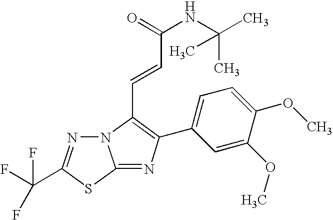 | 500 | 100 |
| 0142 | 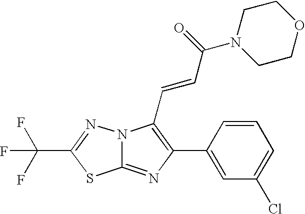 | 500 | 100 |
| 0148 | 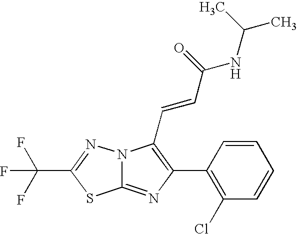 | 500 | 83 |

### Example No. C

### Spodoptera frugiperda Test

### SPODFR Spray Treatment

| | | |
|---|---|---|
| Solvents: | 78 | parts by weight of acetone |
| | 1.5 | parts by weight of dimethylformamide |
| Emulsifier: | 0.5 | part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Discs of maize leaves (Zea mays) are sprayed with a preparation of active compound of the desired concentration and, after drying, populated with caterpillars of the army worm (Spodoptera frugiperda).

After the desired period of time, the effect in % is determined. 100% means that all caterpillars have been killed; 0% means that none of the caterpillars have been killed.

In this test, for example, the following compounds of the Preparation Examples show good activity: see Table 5.

TABLE 5

| | | SPODFR | |
|---|---|---|---|
| | | spray treatment | |
| Example | Structure | g a. i./ha | % 7 d |
| 0585 | 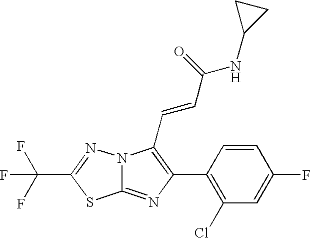 | 500 | 100 |
| 0590 | 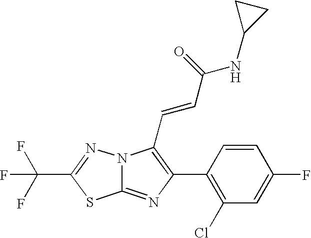 | 500 | 100 |
| 0614 | 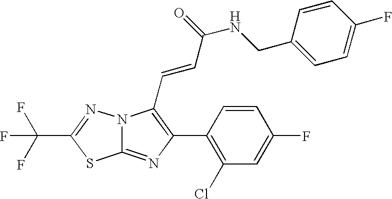 | 500 | 100 |
| 0553 | 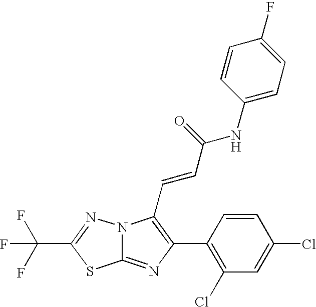 | 500 | 100 |
| 0201 | 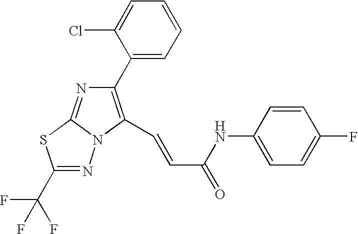 | 500 | 100 |
| 0624 | 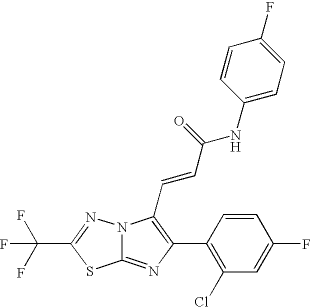 | 500 | 100 |
| 0733 | 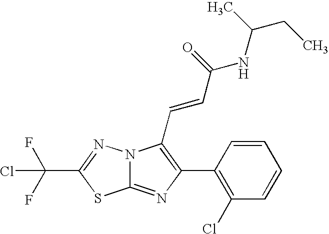 | 500 | 100 |
| 0740 | 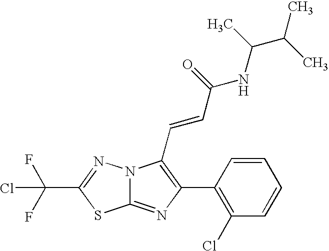 | 500 | 100 |
| 0781 | 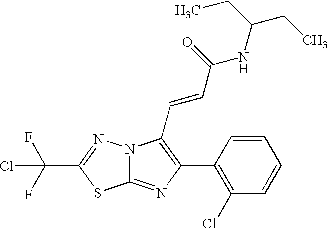 | 500 | 100 |
| 0734 | 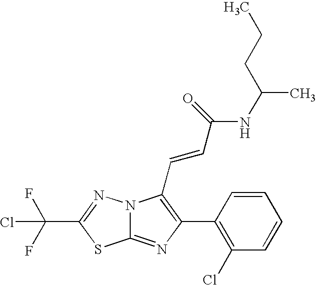 | 500 | 100 |
| 0728 | 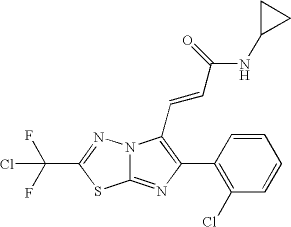 | 500 | 100 |
| 0724 | 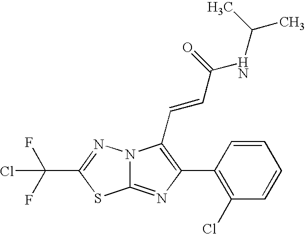 | 500 | 100 |
| 0741 | 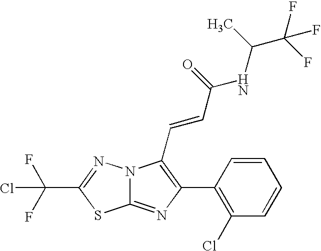 | 500 | 100 |
| 0603 | 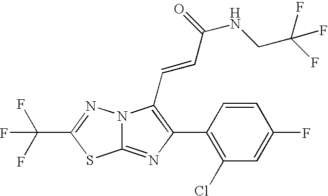 | 500 | 100 |
| 0604 | 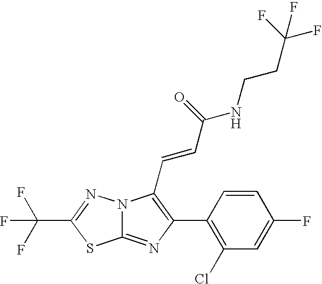 | 500 | 100 |
| 0599 | 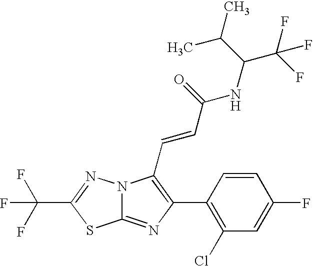 | 500 | 100 |
| 0610 | 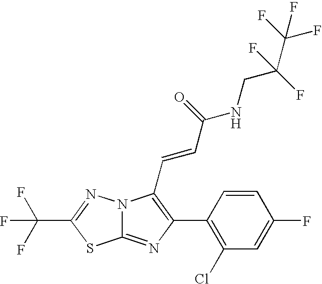 | 500 | 100 |
| 0608 | 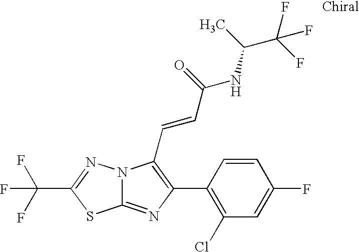 | 500 | 100 |
| 0223 | 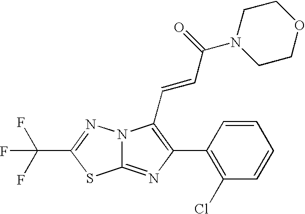 | 500 | 100 |
| 0245 | 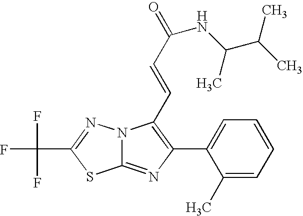 | 500 | 83 |
| 0317 | 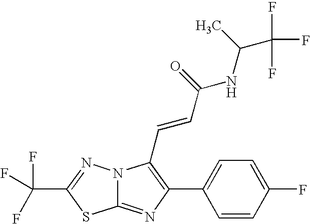 | 500 | 100 |
| 0246 | 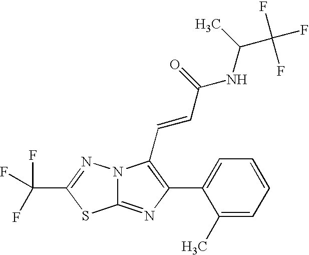 | 500 | 100 |
| 0792 | 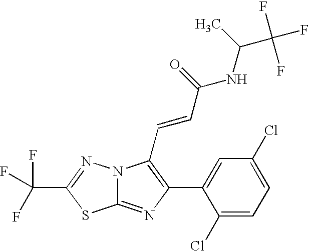 | 500 | 100 |
| 0024 | 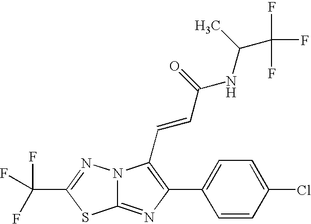 | 500 | 100 |
| 0499 | 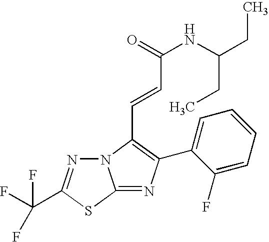 | 500 | 83 |
| 0451 | 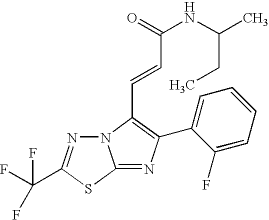 | 500 | 100 |
| 0459 | 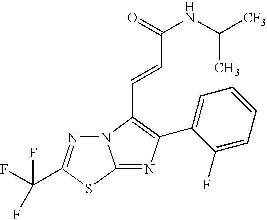 | 500 | 100 |
| 0094 | 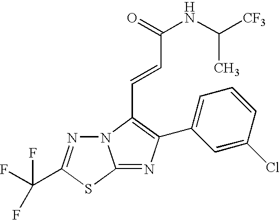 | 500 | 100 |
| 0153 | 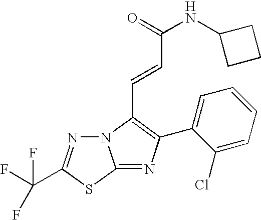 | 500 | 100 |
| 0174 | 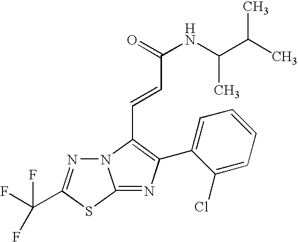 | 500 | 83 |
| 0215 | 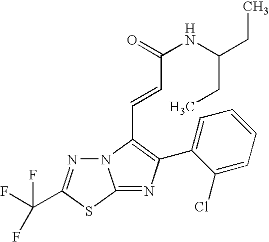 | 500 | 100 |
| 0167 | 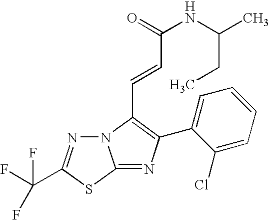 | 500 | 100 |
| 0175 | 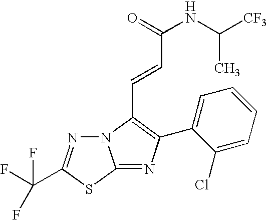 | 500 | 100 |
| 0527 | 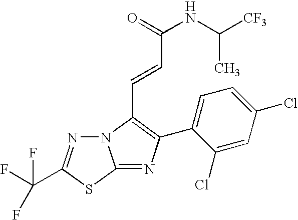 | 500 | 100 |
| 0598 | 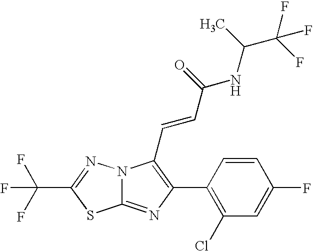 | 500 | 100 |
| 0203 | 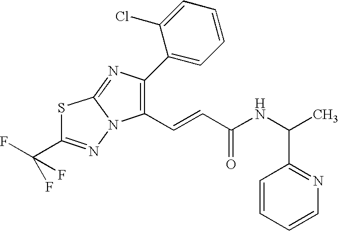 | 500 | 100 |
| 0515 | 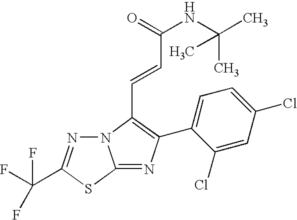 | 500 | 100 |
| 0238 | 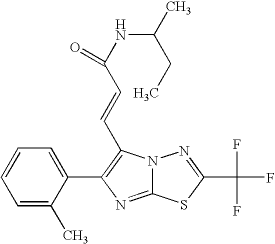 | 500 | 100 |
| 0286 | 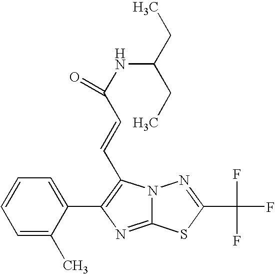 | 500 | 100 |
| 0581 | 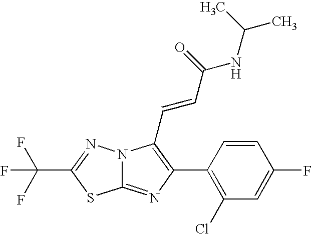 | 500 | 100 |
| 0148 | 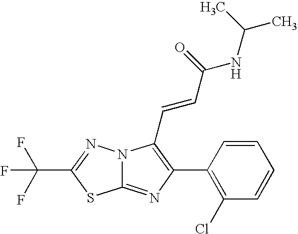 | 500 | 100 |
| 0573 | 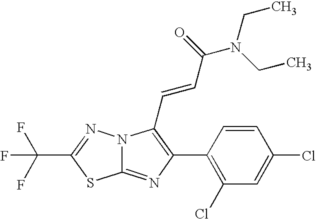 | 500 | 100 |
| 0513 | 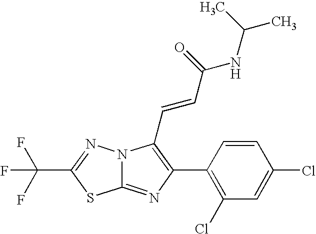 | 500 | 100 |
| 0442 | 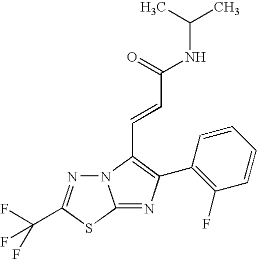 | 500 | 100 |
| 0229 | 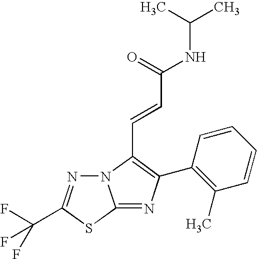 | 500 | 100 |
| 0300 | 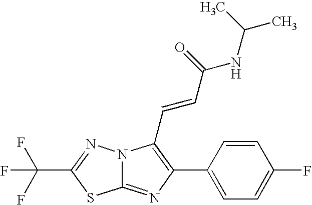 | 500 | 100 |
| 0767 | 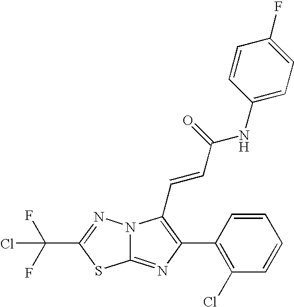 | 500 | 100 |
| 0592 | 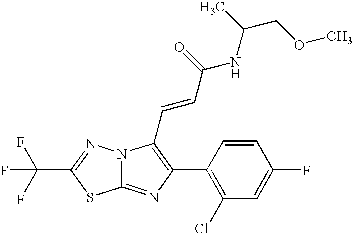 | 500 | 100 |
| 0583 | 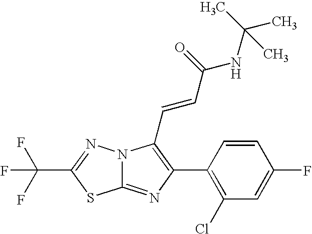 | 500 | 100 |
| 0638 | 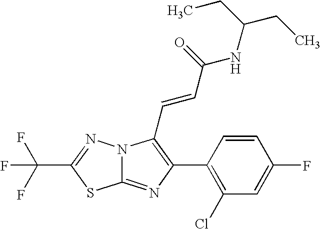 | 500 | 100 |
| 0591 | 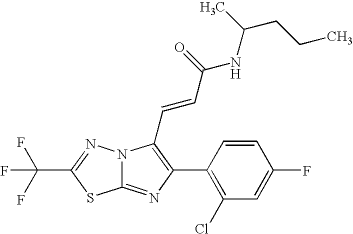 | 500 | 100 |
| 0597 | 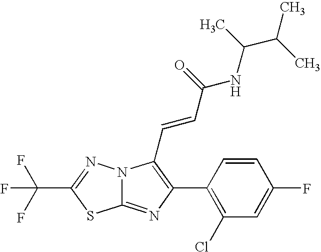 | 500 | 100 |
| 0239 | 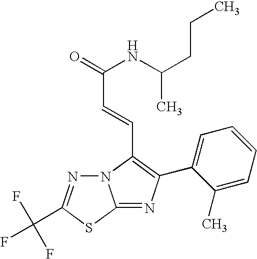 | 500 | 100 |
| 0452 | 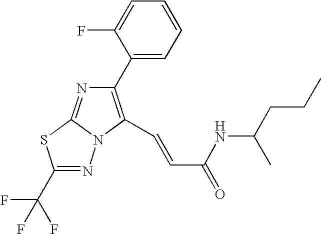 | 500 | 100 |
| 0168 | 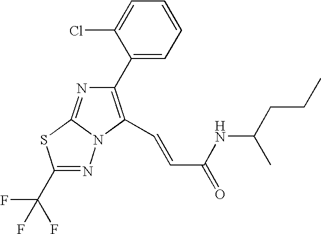 | 500 | 100 |
| 0274 | 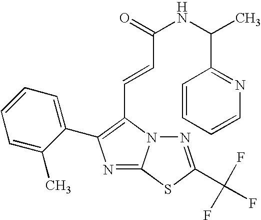 | 500 | 100 |

### Fungicidal Action

### Example No. D

### Sphaerotheca Test (Cucumber)/Protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the preparation of active compound at the stated application rate. 1 day after the treatment, the plants are inoculated with a spore suspension of Sphaerotheca fuliginea. The plants are then placed in a greenhouse at 70% relative atmospheric humidity and a temperature of 23° C.

Evaluation is carried out 7 days after the inoculation. 0% means an efficacy which corresponds to that of the control, whereas an efficacy of 100% means that no infection is observed.

TABLE 6
Sphaerotheca test (cucumber)/protective

| | Active compound | |
|---|---|---|
| Active compound | application rate in ppm | Efficacy in % |
| Example 0912 | 500 | 95 |
| 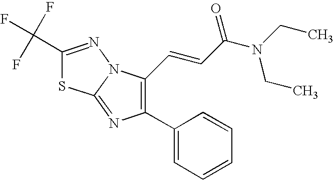 | | |
| Example 0913 | 500 | 90 |
| 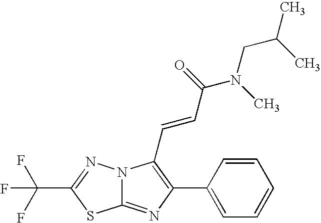 | | |
| Example 0067 | 500 | 90 |
| 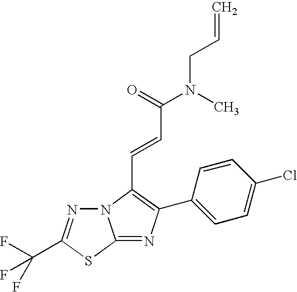 | | |

### Example No. E

### Botrytis Test (Bean)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity. 2 days after the inoculation, the size of the infected areas on the leaves is evaluated. 0% means an efficacy which corresponds to that of the control, whereas an efficacy of 100% means that no infection is observed.

TABLE 7
Botrytis test (bean)/protective

| | Active compound | |
|---|---|---|
| Active compound | application rate in ppm | Efficacy in % |
| Example 0912 | 500 | 93 |
| 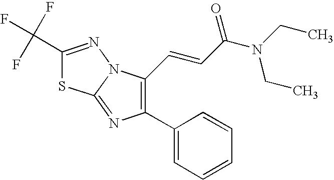 | | |

1. A method of controlling animal pests or treating plant diseases, comprising contacting animal pests, plants or plant propagation material with a compound of formula (I) 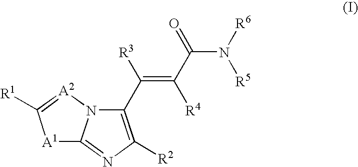
in which
A¹ represents sulphur, oxygen, NH or N-alkyl,
A² represents nitrogen, or carbon which is substituted by hydrogen, halogen, hydroxy, in each case straight-chain, branched or halogenated alkyl, optionally halogenated alkoxy, or optionally halogenated cycloalkyl,
R¹ represents hydrogen, halogen, hydroxy, cyano, or nitro, represents optionally halogen-, hydroxy-, cyano-, alkyl-, haloalkyl-, alkylamino-, dialkylamino- or alkoxy-substituted cycloalkyl, aryl, or heteroaryl, represents optionally halogen-, hydroxy-, cyano-, alkylamino-, dialkylamino- or alkoxy-substituted alkyl, or represents optionally halogen-, cyano-, alkylamino- or dialkylamino-substituted alkoxy, alkylthio, alkylsulphinyl or alkylsulphonyl,
R² represents an aromatic or heteroaromatic radical which is substituted with one or more substituents selected from the group consisting of halogen, cyano, nitro, alkylamino, dialkylamino, alkyl, haloalkyl, alkoxy and phenoxy,
R³ represents hydrogen, alkyl or trifluoromethyl,
R⁴ represents hydrogen, cyano, trifluoromethyl, alkyl, halogen, alkoxy, nitro or SOₙ-alkyl, where n represents an integer from 0 to 2; straight-chain, branched or cyclic alkyl; or COR, where R represents hydrogen, hydroxy, alkyl, alkoxy, aralkoxy, aryloxy or NR′R″ where R′ and R″ have the definitions given for R⁵ and R⁶,
R⁵ and R⁶ are the same or different and represent hydrogen, optionally substituted aryl, optionally substituted heteroaryl; a straight-chain, branched or cyclic, saturated or unsaturated aliphatic hydrocarbon radical which is optionally interrupted by O, S, N-alkyl, NH, N-aryl or N-aralkyl and which is optionally substituted by hydroxy, alkoxy, alkylmercapto, alkylsulphinyl, alkylsulphonyl, trialkylsilyl, alkyl, haloalkyl, halogen, optionally substituted phenyl, optionally substituted heteroaryl, alkoxycarbonyl, alkylamino or dialkylamino, where the alkyl groups in the dialkylamino group together with the N-atom optionally form a 5- to 7-membered ring which is optionally interrupted by a heteroatom selected from the group consisting of O, S, NH and N-alkyl, and where the alkylamino and dialkylamino radicals are optionally substituted by halogen, aryl, aralkyl, alkoxy, alkyl, haloalkyl, alkylmercapto or SO₂-alkyl,
or
R⁵ and R⁶ together with the nitrogen atom to which they are attached, form a 3- to 7-membered saturated or unsaturated ring which optionally contains 1 or 2 heteroatoms selected from the group consisting of oxygen, sulphur and nitrogen, where the nitrogen is optionally substituted by hydrogen, alkyl, aryl or aralkyl, and where the 3- to 7-membered ring is optionally substituted by 1 to 4 identical or different substituents selected from the group consisting of alkyl, halogen, aryl, aralkyl, alkoxycarbonyl, hydroxyalkyl, alkoxyalkyl and trifluoromethyl, or where the 3- to 7-membered ring is optionally fused to an optionally substituted aromatic ring.

2. The method and according to claim 1
in which
A¹ represents sulphur, oxygen or NH,
A² represents nitrogen, or represents carbon which is substituted by hydrogen, halogen, hydroxy, optionally mono- or polyhalogenated alkyl, optionally mono- or polyhalogenated alkoxy, or optionally mono- or polyhalogenated cycloalkyl,
R¹ represents hydrogen, halogen, hydroxy, cyano, nitro, represents optionally halogen-, hydroxy-, cyano-, alkyl-, haloalkyl-, alkylamino-, dialkylamino- or alkoxy-substituted cycloalkyl, represents optionally halogen-, hydroxy-, cyano-, alkylamino-, dialkylamino- or alkoxy-substituted alkyl or represents optionally halogen-, cyano-, alkylamino- or dialkylamino-substituted alkoxy, alkylthio, alkylsulphinyl or alkylsulphonyl,
or
R¹ represents phenyl which is optionally substituted by one to five substituents selected from the group consisting of halogen, hydroxy, cyano, alkyl, alkoxy, haloalkyl, alkylamino and dialkylamino,
or
R¹ represents heteroaryl which is optionally substituted by one to four substituents selected from the group consisting of halogen, hydroxy, cyano, alkyl, alkoxy, haloalkyl, alkylamino and dialkylamino,
R² represents phenyl which is optionally substituted by one to five substituents selected from the group consisting of halogen, cyano, alkyl, alkoxy, phenoxy, haloalkyl, alkylamino and dialkylamino; or heteroaryl which is substituted by one to four substituents selected from the group consisting of halogen, cyano, alkyl, alkoxy, phenoxy, haloalkyl, alkylamino and dialkylamino,
R³ represents hydrogen, trifluoromethyl or (C₁-C₆)-alkyl,
R⁴ represents hydrogen, cyano, trifluoromethyl, (C₁-C₆)-alkyl, nitro, CO—(C₁-C₆)-alkyl, or SO₂(C₁-C₆)-alkyl,
R⁵ and R⁶ are the same or different and represent hydrogen, alkyl, alkenyl, alkynyl, cycloalkyl, cycloalkylalkyl, alkoxyalkyl, di(alkoxy)-alkyl, alkylthioalkyl, alkylsulphinylalkyl, alkylsulphonylalkyl, alkylaminoalkyl, dialkylaminoalkyl, trialkylsilylalkyl, haloalkyl, haloalkenyl, haloalkynyl, halocycloalkyl, halocyloalkylalkyl, or haloalkoxyalkyl; represent aryl, aralkyl, arylhaloalkyl, aryloxyalkyl, heteroaryl or heteroarylalkyl, each of which is substituted by one or more substituents selected from the group consisting of hydrogen, halogen, cyano, nitro, alkylamino, dialkylamino, haloalkyl, alkyl, haloalkoxy and alkoxy; or represent alkoxy, cycloalkoxy, cycloalkylalkoxy or arylalkoxy,
or
R⁵ and R⁶ together with the nitrogen atom to which they are attached, represent a 3- to 7-membered saturated or unsaturated ring which optionally contains 1 or 2 heteroatoms selected from the group consisting of oxygen, sulphur and nitrogen, where the nitrogen is optionally substituted by hydrogen, (C₁-C₆)-alkyl, aryl or aralkyl and where the 3- to 7-membered ring is optionally substituted by 1 to 4 identical or different substituents selected from the group consisting of (C₁-C₆)-alkyl, (C₁-C₆)-haloalkyl, halogen, aryl, aralkyl, (C₁-C₆)-alkoxycarbonyl, and (C₁-C₆)-alkoxyalkyl, or where the 3- to 7-membered ring is optionally fused to an optionally substituted aromatic ring.

3. The method according to claim 1
in which
A¹ represents sulphur or oxygen,
A² represents nitrogen, or represents carbon which is substituted by hydrogen, halogen, hydroxy, optionally mono- or polyhalogenated (C₁-C₆)-alkyl, optionally mono- or polyhalogenated (C₁-C₆)-alkoxy or optionally mono- or polyhalogenated (C₃-C₆)-cycloalkyl,
R¹ represents hydrogen, halogen, hydroxy, cyano, nitro, represents in each case optionally halogen-, cyano-, (C₁-C₃)-alkylamino- and (C₁-C₃)-dialkylamino-substituted methoxy, ethoxy, isopropoxy, or represents methylthio, methylsulphinyl, methylsulphonyl, ethylthio, ethylsulphinyl, ethylsulphonyl, methyl, ethyl, isopropyl, n-propyl, tert-butyl, methoxymethyl, methoxyethyl, cyclopropyl, cyclobutyl, cyclopentyl, cyclohexyl, fluoromethyl, chloromethyl, difluoromethyl, dichloromethyl, trifluoromethyl, difluorochloromethyl, trichloromethyl, fluorodichloromethyl, pentafluoroethyl, 2,2,2-trifluoroethyl, 1,1,1,3,3,3-hexafluoro-2-propyl, heptafluoroisopropyl or 1-chloro-1-cyclopropyl
or
R¹ represents phenyl which is optionally substituted by one to five substituents selected from the group consisting of halogen, hydroxy, cyano, alkyl, alkoxy, (C₁-C₃)-haloalkyl, (C₁-C₃)-alkylamino and (C₁-C₃)-dialkylamino,
or
R¹ represents a five- or six-membered heteroaryl which is optionally substituted by one to four substituents selected from the group consisting of halogen, hydroxy, cyano, (C₁-C₃)-alkyl, (C₁-C₃)-alkoxy, (C₁-C₃)-haloalkyl, (C₁-C₃)-alkylamino and (C₁-C₃)-dialkylamino,
R² represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 3-methylphenyl, 4-methylphenyl, 2-chloro-4-methylphenyl, 2-methyl-4-chlorophenyl, 4-fluorophenyl, 2-methyl-4-fluorophenyl, 3,4-dichlorophenyl, 3,5-dichlorophenyl, 2,3-dichlorophenyl, 2,5-dichlorophenyl, 3,4-difluorophenyl, 3,5-difluorophenyl, 2,3-difluorophenyl, 2,5-difluorophenyl, 2,6-dichlorophenyl, 2,6-difluorophenyl, 2,3,4-trifluorophenyl, 3,4-bis(methoxy)phenyl, 4-methoxyphenyl, 3-methoxyphenyl, 2-methoxyphenyl, 2-chloro-4-methoxyphenyl, 2-fluoro-4-methoxyphenyl, 2-methyl-4-methoxyphenyl, 4-phenoxyphenyl, 2-chloro-4-phenoxyphenyl, 2-fluoro-4-phenoxyphenyl, 2-methyl-4-phenoxyphenyl, 4-(trifluoromethoxy)phenyl, 2-(trifluoromethyl)phenyl, 3-(trifluoromethyl)phenyl, 4-(trifluoromethyl)phenyl, 2-chloro-4-(trifluoromethyl)phenyl, 2-fluoro-4-(trifluoromethyl)phenyl, 4-(tert-butyl)phenyl, 4-cyanophenyl, thienyl, 3-chlorothien-2-yl, 4-chlorothien-2-yl, 5-chlorothien-2-yl, 2-chlorothien-4-yl, 3-chlorothien-4-yl or 2-chlorothien-3-yl, thiazol-2-yl, 5-chloro-4-thiazolyl, 2-chloro-3-pyridyl, 2-chloro-5-pyridyl, or 3-bromo-5-pyridyl,
R³ represents hydrogen,
R⁴ represents hydrogen or cyano,
R⁵ and R⁶ are the same or different and represent hydrogen, (C₁-C₆)-alkyl, (C₂-C₆)-alkenyl, (C₂-C₆)-alkynyl, (C₃-C₆)-cycloalkyl, (C₃-C₆)-cycloalkyl-(C₁-C₄)-alkyl, (C₁-C₆)-alkoxy-(C₁-C₆)-alkyl, di-((C₁-C₆)-alkoxy)-(C₁-C₆)-alkyl, (C₁-C₆)-alkylthio-(C₁-C₆)-alkyl, (C₁-C₆)-alkylsulphinyl-(C₁-C₆)-alkyl, (C₁-C₆)-alkylsulphonyl-(C₁-C₆)-alkyl, (C₁-C₆)-alkylamino-(C₁-C₆)-alkyl, di-(C₁-C₆)-alkylamino-(C₁-C₆)-alkyl, or (tri-(C₁-C₆)-alkyl)silyl-(C₁-C₆)-alkyl; represent (C₁-C₆)-alkyl, (C₂-C₆)-alkenyl, (C₂-C₆)-alkynyl, (C₃-C₆)-cycloalkyl, (C₃-C₆)-cycloalkyl-(C₁-C₄)-alkyl or (C₁-C₆)-alkoxy-(C₁-C₆)-alkyl, each of which is substituted by one or more substituents selected from the group consisting of fluorine, chlorine, bromine and iodine; represent aryl, aryl-(C₁-C₅)-alkyl, aryl-(C₁-C₅)-haloalkyl, aryloxy-(C₁-C₅)-alkyl, heteroaryl or heteroaryl-(C₁-C₅)-alkyl, each of which is substituted by one or more substituents selected from the group consisting of hydrogen, fluorine, chlorine, cyano, nitro, alkylamino, dialkylamino, haloalkyl, alkyl, haloalkoxy and alkoxy; or represent (C₁-C₆)-alkoxy, (C₃-C₆)-cycloalkoxy, (C₃-C₆)-cycloalkyl-(C₁-C₃)-alkoxy or aryl-(C₁-C₆)-alkoxy,
or
R⁵ and R⁶ together with the nitrogen atom to which they are attached, represent in each case optionally fluorine-, chlorine-, bromine-, (C₁-C₆)-alkyl-, trifluoromethyl- or (C₁-C₆)-alkoxy-substituted piperidine, piperazine, morpholine or pyrrolidine.

4. The method according to claim 1
in which
A¹ represents sulphur,
A² represents nitrogen, or represents carbon which is substituted by hydrogen, chlorine, fluorine, bromine, hydroxy, methyl, ethyl, isopropyl, cyclopropyl, methoxy, ethoxy, trifluoromethoxy, trifluoromethyl or 1-chloro-1-cyclopropyl,
R¹ represents hydrogen, fluorine, chlorine, bromine, or hydroxy, represents methoxy, ethoxy, isopropoxy, methylthio, methylsulphinyl, methylsulphonyl, trifluoromethoxy, trichloromethoxy, difluoromethoxy, difluorochloromethoxy, fluorodichloromethoxy, methyl, ethyl, isopropyl, n-propyl, tert-butyl, methoxymethyl, methoxyethyl, cyclopropyl, cyclobutyl, cyclopentyl, cyclohexyl, fluoromethyl, chloromethyl, difluoromethyl, dichloromethyl, trifluoromethyl, difluorochloromethyl, trichloromethyl, fluorodichloromethyl, pentafluoroethyl, 2,2,2-trifluoroethyl, 1,1,1,3,3,3-hexafluoro-2-propyl, heptafluoroisopropyl or 1-chloro-1-cyclopropyl,
or
R¹ represents 4-chlorophenyl or 4-(trifluoromethyl)phenyl,
R² represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 2-chloro-4-methylphenyl, 2-fluoro-4-methylphenyl, 2-methyl-4-chlorophenyl, 4-fluorophenyl, 2-methyl-4-fluorophenyl, 3,4-dichlorophenyl, 2,3-dichlorophenyl, 2,5-dichlorophenyl, 2,3-difluorophenyl, 2,5-difluorophenyl, 2,3,4-trifluorophenyl, 3,4-bis(methoxy)phenyl, 2-chloro-4-methoxyphenyl, 2-fluoro-4-methoxyphenyl, 2-methyl-4-methoxyphenyl, 3-(trifluoromethyl)phenyl, 2-chloro-4-(trifluoromethyl)phenyl, 2-fluoro-4-(trifluoromethyl)phenyl, 3-chlorothien-2-yl, 3-chlorothien-4-yl, 2-chlorothien-3-yl, thiazol-2-yl, 5-chloro-4-thiazolyl, 2-chloro-3-pyridyl, 2-chloro-5-pyridyl, or 3-bromo-5-pyridyl,
R³ represents hydrogen,
R⁴ represents hydrogen and
R⁵ and R⁶ are the same or different and represent hydrogen, methyl, ethyl, n-propyl, isopropyl, n-butyl, sec-butyl, isobutyl, tert-butyl, allyl, propargyl, cyclopropyl, cyclobutyl, cyclopentyl, cyclohexyl, cyclopropylmethyl, 1-pentyl, 2-pentyl, 3-pentyl, 2,2-dimethyl-1-propyl, 3-methyl-1-butyl, 3-dimethyl-1-butyl, 1,1-dimethyl-1-propyl, 1-methoxy-2-propyl, 2-methoxypropyl, 1-ethoxy-2-propyl, 2-ethoxypropyl, 2-methoxyethyl, 2-ethoxyethyl, 2-(methylthio)ethyl, 2-(ethylthio)ethyl, 2-(dimethylamino)ethyl, 2-(diethylamino)ethyl, 1-(dimethylamino)-2-propyl, 1-(diethylamino)-2-propyl, 1-(trimethylsilyl)ethyl, rac-1-methylthio-2-propyl, (S)-1-methylthio-2-propyl, (R)-1-methylthio-2-propyl, rac-1-methylsulphinyl-2-propyl, rac-2,2,2-trifluoromethyl-1-phenethyl, rac-2,2,2-trifluoromethyl-1-(4′-chloro)phenethyl, rac-2,2,2-trifluoromethyl-1-(4′-fluoro)phenethyl, 4,4,4-trifluoro-1-butyl, 1,1,1-trifluoro-2-methyl-2-propyl, (S)-1-methylsulphinyl-2-propyl, (R)-1-methylsulphinyl-2-propyl, rac-1-methylsulphonyl-2-propyl, (S)-1-methylsulphonyl-2-propyl, (R)-1-methylsulphonyl-2-propyl, 3-methyl-2-butyl, 2,2-bis(methoxy)ethyl, rac-1,1,1-trifluoro-2-propyl, (R)-1,1,1-trifluoro-2-propyl, 1,1,1-trifluoro-2-butyl, (S)-1,1,1-trifluoro-2-propyl, 1,1,1-trifluoro-3-methyl-2-butyl, 1,1,1-trifluoro-2-methyl-2-propyl, 1-(trifluoromethyl)-1-cyclopropyl, 2,2,2-trifluoroethyl, 3,3,3-trifluoro-1-propyl, 1,1,1,3,3,3-hexafluoro-2-propyl, heptafluoroisopropyl, 2,2,3,3,3-pentafluoro-1-propyl, 2,2,3,3,4,4,4-heptafluoro-1-butyl, 2-(trifluoromethyl)cyclohexyl, 3-(trifluoromethyl)cyclohexyl, 4-(trifluoromethyl)cyclohexyl, 1,1,1-trifluoro-3-phenyl-3-propyl, benzyl, 4-fluorobenzyl, 4-chlorobenzyl, 1-phenethyl, 1-(4-fluoro)phenethyl, 1-(4-chloro)-phenethyl, 1-phenylpropyl, 1-(4-fluoro)phenylpropyl, 1-(4-chloro)phenylpropyl, 2-phenoxyethyl, 2-(4-fluoro) phenoxyethyl, 2-(4-chloro)phenoxyethyl, 1-phenoxy-2-propyl, 1-(4-fluoro)phenoxy-2-propyl, 1-(4-chloro)phenoxy-2-propyl, 1-cyclopropylethyl, 1-cyclobutylethyl, 1-cyclopentylethyl, 1-cyclohexylethyl, 1-cyclopropylpropyl, 1-cyclobutylpropyl, 1-cyclopentylpropyl, 1-cyclohexylpropyl, 2-phenethyl, 2-(4-fluoro)phenethyl, 2-(4-chloro) phenethyl, phenyl, 4-fluorophenyl, 4-chlorophenyl, 3-chlorophenyl, 2-chlorophenyl, 2,4-dichlorophenyl, 4-trifluoromethoxyphenyl, 4-trifluoromethylphenyl, (2-pyridyl)methyl, (2-pyridyl)-1-ethyl, (3-pyridyl)methyl, (3-pyridyl)-1-ethyl, (4-pyridyl)methyl, (4-pyridyl)-1-ethyl, (5-chloro-2-pyridyl)methyl, (5-chloro-2-pyridyl)-1-ethyl, [3-(trifluoromethyl)-5-chloro-2-pyridyl]-1-methyl, [3-(trifluoromethyl)-5-chloro-2-pyridyl]-1-ethyl, (2-chloro-5-pyridyl)methyl, (2-chloro-5-pyridyl)-1-ethyl, methoxy, ethoxy, isopropoxy, cyclopropylmethoxy, benzyloxy, 1-phenethoxy or 2-phenethoxy,
or
R⁵ and R⁶ together with the nitrogen atom to which they are attached, represent piperidine, piperazine, N-methylpiperazine, morpholine, 2,6-dimethylmorpholine, 2-(trifluoromethyl)piperidine, 3-(trifluoromethyl)piperidine, 4-(trifluoro-methyl)piperidine or pyrrolidine.

5. A compound of the formula (IA) 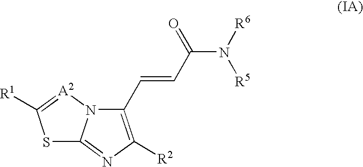
in which
A² represents nitrogen, or represents carbon which is substituted by hydrogen, chlorine, fluorine, bromine, hydroxy, methyl, ethyl, isopropyl, cyclopropyl, methoxy, ethoxy, trifluoromethoxy, trifluoromethyl or 1-chloro-1-cyclopropyl,
R¹ represents hydrogen, fluorine, chlorine, bromine, hydroxy, methoxy, ethoxy, isopropoxy, methylthio, methylsulphinyl, methylsulphonyl, trifluoromethoxy, trichloromethoxy, difluoromethoxy, difluorochloromethoxy, fluorodichloromethoxy, methyl, ethyl, isopropyl, n-propyl, tert-butyl, methoxymethyl, methoxyethyl, cyclopropyl, cyclobutyl, cyclopentyl, cyclohexyl, fluoromethyl, chloromethyl, difluoromethyl, dichloromethyl, trifluoromethyl, difluorochloromethyl, trichloromethyl, fluorodichloromethyl, pentafluorethyl, 2,2,2-trifluoroethyl, 1,1,1,3,3,3-hexafluoro-2-propyl, heptafluoroisopropyl, 1-chloro-1-cyclopropyl or 4-(trifluoromethyl)phenyl,
R² represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 3-methylphenyl, 4-methylphenyl, 2-chloro-4-methylphenyl, 2-fluoro-4-methylphenyl, 2-methyl-4-chlorophenyl, 4-fluorophenyl, 2-methyl-4-fluorophenyl, 3,4-dichlorophenyl, 3,5-dichlorophenyl, 2,3-dichlorophenyl, 2,5-dichlorophenyl, 3,4-difluorophenyl, 3,5-difluorophenyl, 2,3-difluorophenyl, 2,5-difluorophenyl, 2,6-dichlorophenyl, 2,6-difluorophenyl, 2,3,4-trifluorophenyl, 3,4-bis(methoxy)phenyl, 4-methoxyphenyl, 3-methoxyphenyl, 2-methoxyphenyl, 2-chloro-4-methoxyphenyl, 2-fluoro-4-methoxyphenyl, 2-methyl-4-methoxyphenyl, 4-phenoxyphenyl, 2-chloro-4-phenoxyphenyl, 2-fluoro-4-phenoxyphenyl, 2-methyl-4-phenoxyphenyl, 4-(trifluoromethoxy)phenyl, 2-(trifluoromethyl)phenyl, 3-(trifluoromethyl)phenyl, 4-(trifluoromethyl)phenyl, 2-chloro-4-(trifluoromethyl)phenyl, 2-fluoro-4-(trifluoromethyl)phenyl, 4-(tert-butyl)phenyl, 4-cyanophenyl, thienyl, 3-chlorothien-2-yl, 4-chlorothien-2-yl, 5-chlorothien-2-yl, 2-chlorothien-4-yl, 3-chlorothien-4-yl or 2-chlorothien-3-yl, thiazol-2-yl, 5-chloro-4-thiazolyl, 2-chloro-3-pyridyl, 2-chloro-5-pyridyl, or 3-bromo-5-pyridyl,
R⁵ and R⁶ are the same or different and represent hydrogen, methyl, ethyl, n-propyl, isopropyl, n-butyl, sec-butyl, isobutyl, tert-butyl, allyl, propargyl, cyclopropyl, cyclobutyl, cyclopentyl, cyclohexyl, cyclopropylmethyl, 1-pentyl, 2-pentyl, 3-pentyl, 2,2-dimethyl-1-propyl, 3-methyl-1-butyl, 3-dimethyl-1-butyl, 1,1-dimethyl-1-propyl, 1-methoxy-2-propyl, 2-methoxy-propyl, 1-ethoxy-2-propyl, 2-ethoxypropyl, 2-methoxyethyl, 2-ethoxyethyl, 2-(methylthio)ethyl, 2-(ethylthio)ethyl, 2-(dimethylamino)ethyl, 2-(diethylamino)ethyl, 1-(dimethylamino)-2-propyl, 1-(diethylamino)-2-propyl, 1-(trimethylsilyl)ethyl, rac-1-methylthio-2-propyl, (S)-1-methylthio-2-propyl, (R)-1-methylthio-2-propyl, rac-1-methylsulphinyl-2-propyl, rac-2,2,2-trifluoromethyl-1-phenethyl, rac-2,2,2-trifluoromethyl-1-(4′-chloro)phenethyl, rac-2,2,2-trifluoromethyl-1-(4′-fluoro)phenethyl, 4,4,4-trifluoro-1-butyl, 1,1,1-trifluoro-2-methyl-2-propyl, (S)-1-methylsulphinyl-2-propyl, (R)-1-methylsulphinyl-2-propyl, rac-1-methylsulphonyl-2-propyl, (S)-1-methylsulphonyl-2-propyl, (R)-1-methylsulphonyl-2-propyl, 3-methyl-2-butyl, 2,2-bis(methoxy)ethyl, rac-1,1,1-trifluoro-2-propyl, (R)-1,1,1-trifluoro-2-propyl, 1,1,1-trifluoro-2-butyl, (S)-1,1,1-trifluoro-2-propyl, 1,1,1-trifluoro-3-methyl-2-butyl, 1,1,1-trifluoro-2-methyl-2-propyl, 1-(trifluoromethyl)-1-cyclopropyl, 2,2,2-trifluoroethyl, 3,3,3-trifluoro-1-propyl, 1,1,1,3,3,3-hexafluoro-2-propyl, heptafluoroisopropyl, 2,2,3,3,3-pentafluoro-1-propyl, 2,2,3,3,4,4,4-heptafluoro-1-butyl, 2-(trifluoromethyl)cyclohexyl, 3-(trifluoromethyl)cyclohexyl, 4-(trifluoromethyl)cyclohexyl, 1,1,1-trifluoro-3-phenyl-3-propyl, benzyl, 4-fluorobenzyl, 4-chlorobenzyl, 1-phenethyl, 1-(4-fluoro)phenethyl, 1-(4-chloro)-phenethyl, 1-phenylpropyl, 1-(4-fluoro)phenylpropyl, 1-(4-chloro)phenylpropyl, 2-phenoxyethyl, 2-(4-fluoro)phenoxyethyl, 2-(4-chloro)phenoxyethyl, 1-phenoxy-2-propyl, 1-(4-fluoro)phenoxy-2-propyl, 1-(4-chloro)phenoxy-2-propyl, 1-cyclopropylethyl, 1-cyclobutylethyl, 1-cyclopentylethyl, 1-cyclohexylethyl, 1-cyclopropylpropyl, 1-cyclobutylpropyl, 1-cyclopentylpropyl, 1-cyclohexylpropyl, 2-phenethyl, 2-(4-fluoro)phenethyl, 2-(4-chloro)phenethyl, phenyl, 4-fluorophenyl, 4-chlorophenyl, 3-chlorophenyl, 2-chlorophenyl, 2,4-dichlorophenyl, 4-trifluoromethoxyphenyl, 4-trifluoromethylphenyl, (2-pyridyl)methyl, (2-pyridyl)-1-ethyl, (3-pyridyl)methyl, (3-pyridyl)-1-ethyl, (4-pyridyl)methyl, (4-pyridyl)-1-ethyl, (5-chloro-2-pyridyl)methyl, (5-chloro-2-pyridyl)-1-ethyl, [3-(trifluoromethyl)-5-chloro-2-pyridyl]-1-methyl, [3-(trifluoromethyl)-5-chloro-2-pyridyl]-1-ethyl, (2-chloro-5-pyridyl)methyl, (2-chloro-5-pyridyl)-1-ethyl, methoxy, ethoxy, isopropoxy, cyclopropylmethoxy, benzyloxy, 1-phenethoxy, or 2-phenethoxy,
or
R⁵ and R⁶ together with the nitrogen atom to which they are attached, represent piperidine, piperazine, N-methylpiperazine, morpholine, 2,6-dimethylmorpholine, 2-(trifluoromethyl)piperidine, 3-(trifluoromethyl)piperidine, 4-(trifluoro-methyl)piperidine or pyrrolidine.

6. A compound according to claim 5
in which
R² represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 2-chloro-4-methylphenyl, 2-fluoro-4-methylphenyl, 2-methyl-4-chlorophenyl, 4-fluorophenyl, 2-methyl-4-fluorophenyl, 3,4-dichlorophenyl, 2,3-dichlorophenyl, 2,5-dichlorophenyl, 2,3-difluorophenyl, 2,5-difluorophenyl, 2,3,4-trifluorophenyl, 3,4-bis(methoxy)phenyl, 2-chloro-4-methoxyphenyl, 2-fluoro-4-methoxyphenyl, 2-methyl-4-methoxyphenyl, 3-(trifluoromethyl)phenyl, 2-chloro-4-(trifluoromethyl)phenyl, 2-fluoro-4-(trifluoromethyl)phenyl, 3-chlorothien-2-yl, 3-chlorothien-4-yl or 2-chlorothien-3-yl, thiazol-2-yl, 5-chloro-4-thiazolyl, 2-chloro-3-pyridyl, 2-chloro-5-pyridyl, or 3-bromo-5-pyridyl.

7. A compound of the formula (IVA) 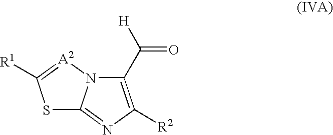
in which
A² represents nitrogen, or represents carbon which is substituted by hydrogen, chlorine, fluorine, bromine, hydroxy, methyl, ethyl, isopropyl, cyclopropyl, methoxy, ethoxy, trifluoromethoxy, trifluoromethyl or 1-chloro-1-cyclopropyl,
R¹ represents hydrogen, fluorine, chlorine, bromine, hydroxy, methoxy, ethoxy, isopropoxy, methylthio, methylsulphinyl, methylsulphonyl, trifluoromethoxy, trichloromethoxy, difluoromethoxy, difluorochloromethoxy, fluorodichloromethoxy, methyl, ethyl, isopropyl, n-propyl, tert-butyl, methoxymethyl, methoxyethyl, cyclopropyl, cyclobutyl, cyclopentyl, cyclohexyl, fluoromethyl, chloromethyl, difluoromethyl, dichloromethyl, trifluoromethyl, difluorochloromethyl, trichloromethyl, fluorodichloromethyl, pentafluorethyl, 2,2,2-trifluoroethyl, 1,1,1,3,3,3-hexafluoro-2-propyl, heptafluoroisopropyl, 1-chloro-1-cyclopropyl or 4-(trifluoromethyl)phenyl,
R² represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 3-methylphenyl, 4-methylphenyl, 2-chloro-4-methylphenyl, 2-fluoro-4-methylphenyl, 2-methyl-4-chlorophenyl, 4-fluorophenyl, 2-methyl-4-fluorophenyl, 3,4-dichlorophenyl, 3,5-dichlorophenyl, 2,3-dichlorophenyl, 2,5-dichlorophenyl, 3,4-difluorophenyl, 3,5-difluorophenyl, 2,3-difluorophenyl, 2,5-difluorophenyl, 2,6-dichlorophenyl, 2,6-difluorophenyl, 2,3,4-trifluorophenyl, 3,4-bis(methoxy)phenyl, 4-methoxyphenyl, 3-methoxyphenyl, 2-methoxyphenyl, 2-chloro-4-methoxyphenyl, 2-fluoro-4-methoxyphenyl, 2-methyl-4-methoxyphenyl, 4-phenoxyphenyl, 2-chloro-4-phenoxyphenyl, 2-fluoro-4-phenoxyphenyl, 2-methyl-4-phenoxyphenyl, 4-(trifluoromethoxy)phenyl, 2-(trifluoromethyl)phenyl, 3-(trifluoromethyl)phenyl, 4-(trifluoromethyl)phenyl, 2-chloro-4-(trifluoromethyl)phenyl, 2-fluoro-4-(trifluoromethyl)phenyl, 4-(tert-butyl)phenyl, 4-cyanophenyl, thienyl, 3-chlorothien-2-yl, 4-chlorothien-2-yl, 5-chlorothien-2-yl, 2-chlorothien-4-yl, 3-chlorothien-4-yl or 2-chlorothien-3-yl, thiazol-2-yl, 5-chloro-4-thiazolyl, 2-chloro-3-pyridyl, 2-chloro-5-pyridyl, or 3-bromo-5-pyridyl.

8. A compound of the formula (VA) 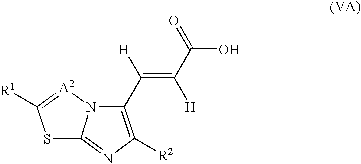
in which
A² represents nitrogen, or represents carbon which is substituted by hydrogen, chlorine, fluorine, bromine, hydroxy, methyl, ethyl, isopropyl, cyclopropyl, methoxy, ethoxy, trifluoromethoxy, trifluoromethyl or 1-chloro-1-cyclopropyl,
R¹ represents hydrogen, fluorine, chlorine, bromine, hydroxy, methoxy, ethoxy, isopropoxy, methylthio, methylsulphinyl, methylsulphonyl, trifluoromethoxy, trichloromethoxy, difluoromethoxy, difluorochloromethoxy, fluorodichloromethoxy, methyl, ethyl, isopropyl, n-propyl, tert-butyl, methoxymethyl, methoxyethyl, cyclopropyl, cyclobutyl, cyclopentyl, cyclohexyl, fluoromethyl, chloromethyl, difluoromethyl, dichloromethyl, trifluoromethyl, difluorochloromethyl, trichloromethyl, fluorodichloromethyl, pentafluorethyl, 2,2,2-trifluoroethyl, 1,1,1,3,3,3-hexafluoro-2-propyl, heptafluoroisopropyl, 1-chloro-1-cyclopropyl or 4-(trifluoromethyl)phenyl,
R² represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 3-methylphenyl, 4-methylphenyl, 2-chloro-4-methylphenyl, 2-fluoro-4-methylphenyl, 2-methyl-4-chlorophenyl, 4-fluorophenyl, 2-methyl-4-fluorophenyl, 3,4-dichlorophenyl, 3,5-dichlorophenyl, 2,3-dichlorophenyl, 2,5-dichlorophenyl, 3,4-difluorophenyl, 3,5-difluorophenyl, 2,3-difluorophenyl, 2,5-difluorophenyl, 2,6-dichlorophenyl, 2,6-difluorophenyl, 2,3,4-trifluorophenyl, 3,4-bis(methoxy)phenyl, 4-methoxyphenyl, 3-methoxyphenyl, 2-methoxyphenyl, 2-chloro-4-methoxyphenyl, 2-fluoro-4-methoxyphenyl, 2-methyl-4-methoxyphenyl, 4-phenoxyphenyl, 2-chloro-4-phenoxyphenyl, 2-fluoro-4-phenoxyphenyl, 2-methyl-4-phenoxyphenyl, 4-(trifluoromethoxy)phenyl, 2-(trifluoromethyl)phenyl, 3-(trifluoromethyl)phenyl, 4-(trifluoromethyl)phenyl, 2-chloro-4-(trifluoromethyl)phenyl, 2-fluoro-4-(trifluoromethyl)phenyl, 4-(tert-butyl)phenyl, 4-cyanophenyl, thienyl, 3-chlorothien-2-yl, 4-chlorothien-2-yl, 5-chlorothien-2-yl, 2-chlorothien-4-yl, 3-chlorothien-4-yl or 2-chlorothien-3-yl, thiazol-2-yl, 5-chloro-4-thiazolyl, 2-chloro-3-pyridyl, 2-chloro-5-pyridyl, or 3-bromo-5-pyridyl.

9. A compound of the formula (VIIA) 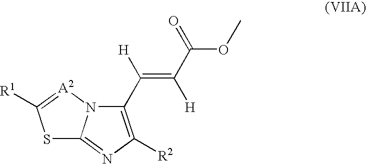
in which
A² represents nitrogen, or represents carbon which is substituted by hydrogen, chlorine, fluorine, bromine, hydroxy, methyl, ethyl, isopropyl, cyclopropyl, methoxy, ethoxy, trifluoromethoxy, trifluoromethyl or 1-chloro-1-cyclopropyl,
R¹ represents hydrogen, fluorine, chlorine, bromine, hydroxy, methoxy, ethoxy, isopropoxy, methylthio, methylsulphinyl, methylsulphonyl, trifluoromethoxy, trichloromethoxy, difluoromethoxy, difluorochloromethoxy, fluorodichloromethoxy, methyl, ethyl, isopropyl, n-propyl, tert-butyl, methoxymethyl, methoxyethyl, cyclopropyl, cyclobutyl, cyclopentyl, cyclohexyl, fluoromethyl, chloromethyl, difluoromethyl, dichloromethyl, trifluoromethyl, difluorochloromethyl, trichloromethyl, fluorodichloromethyl, pentafluorethyl, 2,2,2-trifluoroethyl, 1,1,1,3,3,3-hexafluoro-2-propyl, heptafluoroisopropyl, 1-chloro-1-cyclopropyl or 4-(trifluoromethyl)phenyl,
R² represents 2-bromophenyl, 2-iodophenyl, 2-chlorophenyl, 3-chlorophenyl, 4-chlorophenyl, 2,4-dichlorophenyl, 2-fluorophenyl, 2,4-difluorophenyl, 2-chloro-4-fluorophenyl, 2-fluoro-4-chlorophenyl, 2-methylphenyl, 2-ethylphenyl, 3-methylphenyl, 4-methylphenyl, 2-chloro-4-methylphenyl, 2-fluoro-4-methylphenyl, 2-methyl-4-chlorophenyl, 4-fluorophenyl, 2-methyl-4-fluorophenyl, 3,4-dichlorophenyl, 3,5-dichlorophenyl, 2,3-dichlorophenyl, 2,5-dichlorophenyl, 3,4-difluorophenyl, 3,5-difluorophenyl, 2,3-difluorophenyl, 2,5-difluorophenyl, 2,6-dichlorophenyl, 2,6-difluorophenyl, 2,3,4-trifluorophenyl, 3,4-bis(methoxy)phenyl 4-methoxyphenyl, 3-methoxyphenyl, 2-methoxyphenyl, 2-chloro-4-methoxyphenyl, 2-fluoro-4-methoxyphenyl, 2-methyl-4-methoxyphenyl, 4-phenoxyphenyl, 2-chloro-4-phenoxyphenyl, 2-fluoro-4-phenoxyphenyl, 2-methyl-4-phenoxyphenyl, 4-(trifluoromethoxy)phenyl, 2-(trifluoromethyl)phenyl, 3-(trifluoromethyl)phenyl, 4-(trifluoromethyl)phenyl, 2-chloro-4-(trifluoromethyl)phenyl, 2-fluoro-4-(trifluoromethyl)phenyl, 4-(tert-butyl)phenyl, 4-cyanophenyl, thienyl, 3-chlorothien-2-yl, 4-chlorothien-2-yl, 5-chlorothien-2-yl, 2-chlorothien-4-yl, 3-chlorothien-4-yl or 2-chlorothien-3-yl, thiazol-2-yl, 5-chloro-4-thiazolyl, 2-chloro-3-pyridyl, 2-chloro-5-pyridyl, or 3-bromo-5-pyridyl.

10. A process for preparing a compound of the formula (IA) according to claim 5, comprising
(a) reacting a heterobicyclic acrylic acid of the general formula (VA) 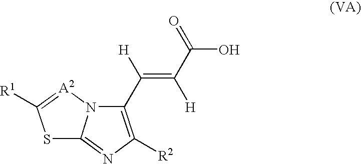
in which A², R¹ and R² are as defined in claim 5 with an amine of the general formula (VIII) 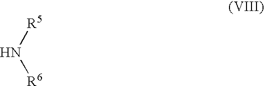
in which R⁵ and R⁶ are as defined in claim 5, optionally in the presence of one or more diluents and optionally in the presence of one or more reaction auxiliaries,
or
(b) reacting a heterobicyclic acrylic acid of the general formula (VA) with a halogenating agent optionally in the presence of one or more diluents and optionally in the presence of one or more reaction auxiliaries; followed by a reaction with an amine of the general formula (VIII) in which R⁵ and R⁶ are as defined in claim 5, optionally in the presence of one or more diluents and optionally in the presence of one or more reaction auxiliaries.

11. A composition for controlling animals pests or treating plant diseases, comprising least one compound of formula (I) according to claim 1 and one or more extenders, surfactants, or combinations thereof.

12. A method for controlling pests, comprising contacting a pest or its habitat with a compound of formula (I) according to claim 1.

13. A method for controlling unwanted microorganisms, comprising contacting a microorganism or its habitat with a compound of formula (I) according to claim 1.

14. A process for preparing a pesticide preparation or a composition for controlling plant diseases, comprising mixing a compound of formula (I) according to claim 1 with one or more extenders, surfactants, or combinations thereof.

15. The method of claim 1, wherein the plant propagating material is a seed.

16. The method of claim 15, wherein the seed is a seed of a transgenic plant.
